(19) United States
(12) Patent Application Publication
(10) Pub. No.: US 2010/0150924 A1
(43) Pub. Date: Jun. 17, 2010

(54) REGULATION OF MYELINATION BY NECTIN-LIKE (NECL) MOLECULES

(76) Inventors: Elior Peles, Rehovot (IL); Ivo Spiegel, Tel-Aviv (IL)

Correspondence Address: FENNEMORE CRAIG, 3003 NORTH CENTRAL AVENUE, SUITE 2600, PHOENIX, AZ, 85012, US

(21) Appl. No.: 12/600,833

(22) PCT Filed: May 22, 2008
(86) PCT No.: PCT/IL08/00696

§ 371 Date: Nov. 18, 2009

Related U.S. Application Data
(60) Provisional application No. 60/939,501, filed on May 22, 2007.

(51) Int. Cl. A61K 39/395; C12N 5/10; C12N 5/02; C12N 15/63; A61K 38/16; A61K 31/7088; C07K 14/435; C07K 16/00; C07H 21/04; C07H 21/02

(52) U.S. Cl. 424/134.1; 435/325; 435/375; 435/320.1; 514/12; 514/44 R; 530/350; 530/387.3; 536/23.4; 536/23.5; 536/24.5

(57) ABSTRACT

The invention provides polypeptides comprising isolated domains of Necl proteins, particularly of Necl4, that mediate axon-glia adhesion required for myelination. The invention further provides pharmaceutical compositions comprising as the active ingredient a polypeptide comprising an isolated Necl4 domain, or a polynucleotide encoding a polypeptide comprising an isolated Necl4 domain. Further provided are antibodies directed against isolated Necl domains, siRNA capable of down regulating Necl4 expression, and methods for treating neurological disorders which are associated with aberrant myelination.

### FIELD OF THE INVENTION

The present invention relates to Necl proteins, including Necl4 and isolated domains thereof, that mediate axon-glia adhesion required for myelination. The invention provides pharmaceutical compositions comprising Necl4 domains or polynucleotides encoding them, as well as antibodies, siRNA and methods for treating neurological disorders which are associated with demyelination.

### BACKGROUND OF THE INVENTION

Schwann cells and oligodendrocytes are the myelin-forming cells of the vertebrate peripheral (PNS) and central (CNS) nervous systems, respectively. Myelin is a multilamellar structure that electrically insulates the axon and allows rapid and efficient nerve conduction. PNS myelination is the last step in the lineage-development of the neural crest-derived Schwann cells. At all stages, this development occurs in a very intimate interaction with the axons of the peripheral nerves: Schwann cells first migrate along the nascent axons, then establish a one-to-one association with them and finally align themselves along these axons and ensheath them before they enter active myelination. Although many of the axonal and glial signals regulating the initial differentiation of Schwann cells are known (Jessen and Mirsky, 2005), the molecular events governing the onset of myelination are poorly understood. In particular, the identity of cell adhesion and recognition molecules (CAMs) that mediate axon-Schwann cell interactions during myelination have hitherto remained elusive.

Genes encoding novel cell-adhesion and signaling molecules expressed during myelination were putatively identified by screening cDNA libraries constructed from rat primary Schwann cells and rat sciatic nerve using a signal-sequence trap technique (Spiegel et al., 2006). Both of these sources are expected to be enriched in mRNAs that might be important for myelination. The predicted protein sequences identified include Necl4 (GenBank Accession No. XM 344870), which is a member of the family of Nectin-like (Necl) molecules. Necls belong to a small group of the immunoglobulin superfamily (Ig-CAMs) that contains four different members (Necl1, Necl2, Necl3 and Necl4) in humans and rodents (Biderer, 2005). Molecules of this subfamily were isolated in recent years from various sources and in different biological contexts, and were therefore given a variety of names i.e. Necl1/IgSF4B/SynCAM3/Tsl11; Necl2/IgSF4A/SynCAM1/Tslc1/RA175/SgIGSF; Necl3/IgSF4D/SynCAM2; and Necl4/IgSF4C/SynCAM4/TSLL2.

All Necl molecules are type I transmembrane proteins that contains three immunoglobulin-like (Ig-like) loops in their extracellular domain, which makes them similar to the Nectin-family of cell-adhesion molecules and to the receptor for the poliovirus (PVR), which was initially termed Necl5. In contrast to PVR/Necl5, each of Necl1, Necl2, Necl3 and Necl4 have a short carboxy-terminal intracellular domain containing a 4.1 protein-binding motif and a type II PDZ domain-binding sequence. The best-described Nectin-like molecule is Necl2 which was originally isolated as a tumor-suppressor gene (Kuramochi et al., 2001), a function that was recently also ascribed to Necl4 (Williams et al., 2006). All of the Necls described so far can mediate Ca²⁺-independent cell-adhesion (Biederer et al., 2002; Kakunaga et al., 2005; Williams et al., 2006). Based on their tissue distribution, subcellular localization, and interactions with scaffolding proteins, it has been proposed that the Necls play an important role in the organization of the plasma membrane at specific areas of cell-cell contact (Kakunaga et al., 2005; Mao et al., 2003; Shingai et al., 2003; Yageta et al., 2002; Zhou et al., 2005).

Hitherto, information regarding the function of Necls in the nervous system has been limited. Necl2 was disclosed to induce the formation of CNS-synapses (Biederer et al., 2002), while Necl1 was disclosed to be located at various non junctional adhesion sites between neurons and glia (Kakunaga et al., 2005).

US Patent Application Publication No. 2003/0109016 discloses the nucleotide sequence and predicted amino acid sequence of human Necl1/IgSF4B/TSLL1 which was identified as a tumor suppressor gene for brain and nerve cell tumors. US Patent Application No. 2003/0109027 discloses the nucleotide sequence and predicted amino acid sequence of human Necl4/IgSF4C/TSLL2, which was identified as a tumor suppressor gene for non-small cell lung cancers.

U.S. Pat. No. 6,576,607 and US Patent Application Publication No. 2005/0222041 relate to methods for promoting remyelination in the central and peripheral nervous systems comprising administering an agent comprising a neural cell adhesion molecule.

A number of debilitating conditions are associated with nerve demyelination or failure of the glial cells to properly ensheath the axons of the PNS and/or CNS. Such conditions include diabetic neuropathy, Guillain-Barre disease (acute demyelinating polyneuropathy), chronic inflammatory demyelinating polyradiculoneuropathy, multiple sclerosis and HIV inflammatory demyelinating disease. Accidental events such as physical injury and exposure to toxins or radiation can also result in demyelination of the PNS or CNS. Thus, there is a need for compositions and methods for enhancing the production of myelin.

The background art does not teach or suggest that particular domains of human Necl4 can influence the extent of myelination of neurons, nor that isolated Necl4 domains can be used for treatment of neurological damage associated with aberrant myelination in the central and/or peripheral nervous system.

### SUMMARY OF THE INVENTION

The present invention for the first time discloses that Nectin-like (Necl) cell adhesion molecules mediate axon-glia interactions required for myelination. Specifically, the present invention discloses that Necl4, the major Necl present on glial cells in the peripheral and central nervous systems, is the receptor of Necl1, present on neuronal axons. Furthermore, Necl4 expression is induced and up-regulated in glial cells by axonal contact, and its expression is dramatically increased during periods of active myelination. The present invention is based in part, on the finding that Necl4, and in particular its cytoplasmic domain and its extracellular domain, each individually influence the extent of myelination in neuronal cells. Hence, without wishing to be bound by any theory or mechanism of action, individual domains of Necl4 have an important role in the development and maintenance of myelinated nerves.

The principles of the invention disclosed herein have been demonstrated in a rat model system using recombinant rat Necl nucleotide and protein molecules. Without being bound to any theory, it is believed that those principles may be applied to the corresponding human system, given the high degree of homology between respective human and rat Ned molecules, for example human Necl4 and rat Necl4, and the established parallel processes of neuronal development among vertebrate species.

The present invention thus provides polypeptides comprising an isolated domain of human Necl4 and pharmaceutical compositions comprising such polypeptides, which can be used for the treatment of neurological damage, including that caused by disorders associated with aberrant myelination. As used herein, “a polypeptide comprising an isolated domain of Necl4” expressly excludes a polypeptide corresponding to the full length amino acid sequence of human Necl4 as set forth in SEQ ID NO:1.

The invention further provides compositions and methods of down regulating Necl4 expression in a cell. Accordingly, down regulation is achieved by an siRNA molecule which can induce down-regulation of Necl4 expression in a cell. Such down regulated cells can be used to analyze the function of Necl4 and specific individual domains thereof. Furthermore, such cells can be used as a model of a neurological defect associated with defective cell adhesion and/or myelination.

According to a first aspect, the invention provides a polypeptide comprising an isolated domain of human Necl4, or a fragment, analog or derivative thereof, wherein the isolated domain of human Necl4 is selected from the group consisting of: the cytoplasmic domain, the extracellular domain and the transmembrane domain. In various embodiments, the cytoplasmic domain has the sequence set forth in SEQ ID NO:4; the extracellular domain has a sequence selected from the group consisting of SEQ ID NO:2 and SEQ ID NO:3, and the transmembrane domain has the sequence set forth in SEQ ID NO:5. In one embodiment, the domain of human Necl4 is selected from the group consisting of: (i) the cytoplasmic domain wherein the polypeptide lacks at least one domain of human Necl4 selected from the group consisting of: the extracellular domain and the transmembrane domain; and (ii) the extracellular domain, wherein the polypeptide lacks at least one domain of human Necl4 selected from the group consisting of: the cytoplasmic domain and the transmembrane domain. In specific embodiments, the polypeptide comprises the extracellular domain of human Necl4 as set forth in SEQ ID NO:2, or the cytoplasmic domain of human Necl4 as set forth in SEQ ID NO:4, or the transmembrane domain of human Necl4, as set forth in SEQ ID NO:5. In one embodiment, the polypeptide substantially lacks the native signal sequence of human Necl4. In one embodiment, the fragment of the extracellular domain of human Necl4 has the sequence set forth in SEQ ID NO:3.

In one embodiment, the isolated polypeptide comprises a chemical modification selected from the group consisting of: glycosylation, pegylation, oxidation, permanent phosphorylation, reduction, myristylation, sulfation, acylation, acetylation, ADP-ribosylation, amidation, hydroxylation, iodination, methylation, and derivatization by blocking groups.

In one embodiment, the derivative of the extracellular domain of human Necl4 has a sequence selected from the group consisting of: SEQ ID NO:6; SEQ ID NO:7; SEQ ID NO:10; SEQ ID NO:11; SEQ ID NO:98 and SEQ ID NO:99. In one embodiment, the derivative of the cytoplasmic domain of human Necl4 has a sequence as set forth in SEQ ID NO:8.

In one embodiment, the polypeptide is a fusion protein further comprising an amino acid sequence derived from a heterologous protein. In one embodiment, the heterologous protein is selected from the group consisting of: an immunoglobulin; a marker protein; a protein associated with neural cells; a heterologous human Necl; and a fragment of any of the aforementioned. In one embodiment, the heterologous protein is an immunoglobulin fragment selected from the group consisting of: Fc, Fab, scFv, dsFv, V_{L} and V_{H}. In one embodiment, the immunoglobulin or immunoglobulin fragment has specificity for a protein associated with neural cells. In one embodiment, the heterologous protein is a protein associated with neural cells. In particular embodiments, the protein associated with neural cells is selected from the group consisting of MBP (myelin basic protein); MAG (myelin associated glycoprotein); Caspr; GFAP (glial fibrillary acidic protein); L1; contactin; neurofascin; TAG-1; Nr-CAM (neuron-glia-related cell-adhesion molecule) and gliomedin. In one embodiment, the heterologous human Necl is selected from the group consisting of: Necl1, Necl2 and Necl3. In one embodiment, the fragment of the heterologous human Necl is an isolated domain thereof. In one embodiment, the isolated domain of the heterologous human Necl is selected from the group consisting of: the extracellular domain of human Necl1 as set forth in SEQ ID NO:17; the cytoplasmic domain of human Necl1 as set forth in SEQ ID NO:18; the transmembrane domain of human Necl1 as set forth in SEQ ID NO:19; the extracellular domain of human Necl2 as set forth in SEQ ID NO:20; the cytoplasmic domain of human Necl2 as set forth in SEQ ID NO:21; the transmembrane domain of human Necl2 as set forth in SEQ ID NO:22; the extracellular domain of human Necl3 as set forth in SEQ ID NO:23; the cytoplasmic domain of human Necl3 as set forth in SEQ ID NO:24, and the transmembrane domain of human Necl3 as set forth in SEQ ID NO:25.

In another embodiment, there is provided a pharmaceutical composition comprising the polypeptide for treating neurological damage

According to another aspect, the present invention provides a pharmaceutical composition comprising as an active ingredient a polypeptide comprising an isolated domain of human Necl4, or a fragment, analog or derivative thereof, wherein the domain of human Necl4 is selected from the group consisting of: the cytoplasmic domain; the extracellular domain, and the transmembrane domain; and a pharmaceutically acceptable carrier. In one embodiment, the domain of human Necl4 is selected from the group consisting of: (i) the cytoplasmic domain wherein the polypeptide lacks at least one domain of human Necl4 selected from the group consisting of: the extracellular domain and the transmembrane domain; and (ii) the extracellular domain, wherein the polypeptide lacks at least one domain of human Necl4 selected from the group consisting of: the cytoplasmic domain and the transmembrane domain. In specific embodiments, the polypeptide comprises the extracellular domain of human Necl4 as set forth in SEQ ID NO:2 or SEQ ID NO:3; or the cytoplasmic domain of human Necl4 as set forth in SEQ ID NO:4, or the transmembrane domain of human Necl4, as set forth in SEQ ID NO:5. In one embodiment, the polypeptide substantially lacks the native signal sequence of human Necl4. In one embodiment, the fragment of the extracellular domain of human Necl4 has the sequence set forth in SEQ ID NO:3.

In one embodiment, the derivative of the extracellular domain of human Necl4 is selected from the group consisting of: SEQ ID NO:6; SEQ ID NO:7; SEQ ID NO:10; SEQ ID NO:11; SEQ ID NO:98 and SEQ ID NO:99. In one embodiment, the derivative of the cytoplasmic domain of human Necl4 has a sequence as set forth in SEQ ID NO:8.

In one embodiment, the polypeptide is a fusion protein further comprising an amino acid sequence derived from a heterologous protein. In one embodiment, the heterologous protein is selected from the group consisting of an immunoglobulin; a marker protein; a protein associated with neural cells; a heterologous human Necl; and a fragment of any of the aforementioned. In one embodiment, the heterologous protein is an immunoglobulin fragment selected from the group consisting of: Fc, Fab, scFv, dsFv, V_{L} and V_{H}. In one embodiment, the immunoglobulin or immunoglobulin fragment has specificity for a protein associated with neural cells. In one embodiment, the heterologous protein is a protein associated with neural cells. In particular embodiments, the protein associated with neural cells is selected from the group consisting of MBP (myelin basic protein); MAG (myelin associated glycoprotein); Caspr; GFAP (glial fibrillary acidic protein); L1; contactin; neurofascin; TAG-1; Nr-CAM (neuron-glia-related cell-adhesion molecule) and gliomedin. In one embodiment, the heterologous human Necl is selected from the group consisting of: Necl1, Necl2 and Necl3. In one embodiment, the fragment of the heterologous human Necl is an isolated domain thereof. In one embodiment, the isolated domain is selected from the group consisting of: the extracellular domain of human Necl1 as set forth in SEQ ID NO:17; the cytoplasmic domain of human Necl1 as set forth in SEQ ID NO:18; the transmembrane domain of human Necl1 as set forth in SEQ ID NO:19; the extracellular domain of human Necl2 as set forth in SEQ ID NO:20; the cytoplasmic domain of human Necl2 as set forth in SEQ ID NO:21; the transmembrane domain of human Necl2 as set forth in SEQ ID NO:22; the extracellular domain of human Necl3 as set forth in SEQ ID NO:23; the cytoplasmic domain of human Necl3 as set forth in SEQ ID NO:24, and the transmembrane domain of human Necl3 as set forth in SEQ ID NO:25.

In one embodiment, the pharmaceutical composition further comprises at least one isolated domain of a heterologous human Necl, or a fragment, analog or derivative thereof, wherein the heterologous human Necl is selected from the group consisting of: Necl1, Necl2 and Necl3. In one embodiment, the domain of a heterologous human Necl is selected from the group consisting of: the extracellular domain of human Necl1 as set forth in SEQ ID NO:17; the cytoplasmic domain of human Necl1 as set forth in SEQ ID NO:18; the transmembrane domain of human Necl1 as set forth in SEQ ID NO:19; the extracellular domain of human Necl2 as set forth in SEQ ID NO:20; the cytoplasmic domain of human Necl2 as set forth in SEQ ID NO:21; the transmembrane domain of human Necl2 as set forth in SEQ ID NO:22; the extracellular domain of human Necl3 as set forth in SEQ ID NO:23; the cytoplasmic domain of human Necl3 as set forth in SEQ ID NO:24, and the transmembrane domain of human Necl3 as set forth in SEQ ID NO:25. In one embodiment, the derivative of the domain of a heterologous human Necl is selected from the group consisting of SEQ ID NOS.:26-36; SEQ ID NO:38; SEQ ID NO:39; SEQ ID NO:41 and SEQ ID NO:42.

In one embodiment, the pharmaceutical composition comprising the polypeptide is for treating neurological damage.

According to another aspect, the present invention provides an isolated polynucleotide sequence encoding a polypeptide domain of human Necl4, or a fragment, analog or derivative thereof, wherein the domain of human Necl4 is selected from the group consisting of: the cytoplasmic domain; the extracellular domain, and the transmembrane domain. In one embodiment, the domain of human Necl4 comprises an amino acid sequence selected from the group consisting of: SEQ ID NO:2, SEQ ID NO:3, SEQ ID NO:4 and SEQ ID NO:5. In one embodiment, the derivative of the domain of human Necl4 is selected from the group consisting of: SEQ ID NO:6; SEQ ID NO:7; SEQ ID NO:8; SEQ ID NO:10, SEQ ID NO:11; SEQ ID NO:98 and SEQ ID NO:99. In one embodiment, the isolated polynucleotide sequence comprises a nucleotide sequence selected from the group consisting of: SEQ ID NO:50; SEQ ID NO:51; SEQ ID NO:52, SEQ ID NO:53; SEQ ID NO:103 and SEQ ID NO:104. According to another aspect, there is provided an expression vector comprising the isolated polynucleotide sequence of the invention. In one embodiment, the expression vector further comprises at least one regulatory element operatively linked to the polynucleotide. Examples of regulatory elements include a promoter, an enhancer, a selectable gene, a signal peptide, a recombinase gene, a transcription factor gene and a reporter gene. In one embodiment, the expression vector has a nucleotide sequence selected from the group consisting of: SEQ ID NO:84; SEQ ID NO:89; SEQ ID NO:91 and SEQ ID NO:95.

According to yet another aspect, the present invention provides a pharmaceutical composition comprising an expression vector comprising an isolated polynucleotide sequence encoding a polypeptide domain of human Necl4, or a fragment, analog or derivative thereof as hereinbefore described, and a pharmaceutically acceptable carrier. In one embodiment, the domain of human Necl4 comprises an amino acid sequence selected from the group consisting of: SEQ ID NO:2, SEQ ID NO:3, SEQ ID NO:4 and SEQ ID NO:5. In one embodiment, the pharmaceutical composition comprising the expression vector is for treating neurological damage. In one embodiment, the isolated polynucleotide sequence comprises a sequence selected from the group consisting of: SEQ ID NO:50; SEQ ID NO:51; SEQ ID NO:52, SEQ ID NO:53; SEQ ID NO:103 and SEQ ID NO:104. In one embodiment, the expression vector has a nucleotide sequence selected from the group consisting of: SEQ ID NO:84; SEQ ID NO:89; SEQ ID NO:91 and SEQ ID NO:95.

In another aspect, the invention provides a host cell expressing an exogenous domain of human Necl4 or a fragment, analog or derivative thereof. In one embodiment, the host cell is transformed with an expression vector comprising a polynucleotide sequence encoding a polypeptide comprising a domain of human Necl4 or a fragment, analog or derivative thereof, wherein the polypeptide comprises an amino acid sequence selected from the group consisting of: SEQ ID NO:2, SEQ ID NO:3, SEQ ID NO:4 and SEQ ID NO:5. In one embodiment, the expression vector has a nucleotide sequences selected from the group consisting of: SEQ ID NO:84; SEQ ID NO:89; SEQ ID NO:91 and SEQ ID NO:95. In one embodiment, the host cell is selected from the group consisting of: a eukaryotic cell, a somatic cell, a germ cell, a neuronal cell, a pluripotent stem cell and a nerve progenitor cell. In yet another embodiment, the neuronal cell is selected from the group consisting of: Schwann cell, myelinating Schwann cell, glial cell and dorsal root ganglion neuron.

In another embodiment, the invention provides a method of treating neurological damage in a subject in need thereof, wherein the method comprises transplanting into the subject the host cells expressing an exogenous domain of human Necl4 or a fragment, analog or derivative thereof. In one embodiment, the transplanted host cells are autologous. In another embodiment, the host cells are transplanted at or near at least one predetermined locus. In another embodiment, the invention provides a pharmaceutical composition comprising host cells expressing an exogenous domain of human Necl4 or a fragment, analog or derivative thereof for treating neurological damage.

In various embodiments of the expression vector and the host cell, the polynucleotide sequence is selected from the group consisting of SEQ ID NO:50; SEQ ID NO:51; SEQ ID NO:52 and SEQ ID NO:53.

In another aspect, the invention provides a method of treating neurological damage in a subject in need thereof, wherein the method comprises administering a pharmaceutical composition comprising a polypeptide comprising an isolated domain of human Necl4 or a fragment, analog or derivative thereof, or wherein the method comprises administering a pharmaceutical composition comprising an expression vector comprising an isolated polynucleotide sequence encoding a polypeptide domain of human Necl4, or a fragment, analog or derivative thereof. The polypeptide comprising the isolated domain of human Necl4, the expression vector and the isolated polynucleotide sequence are as hereinbefore described. In one embodiment, the neurological damage comprises nerve demyelination associated with the peripheral nervous system or the central nervous system. In one embodiment, the nerve demyelination is associated with toxin or radiation exposure. In another embodiment, the neurological damage is associated with a neural injury or disease. Examples of diseases include, but are not limited to acute inflammatory demyelinating polyradiculoneuropathy, chronic inflammatory demyelinating polyradiculoneuropathy, chronic idiopathic axonal polyneuropathy, post infection encephalomyelitis, adrenoleukodystrophy, diabetic neuropathy, Guillain-Barre disease (acute demyelinating polyneuropathy), multiple sclerosis and HIV inflammatory demyelinating disease.

In yet another aspect, the invention provides an siRNA molecule capable of down regulating expression of Necl4 via RNA interference. According to one embodiment, the siRNA molecule is selected from the group consisting of: a double stranded molecule comprising two separate RNA strands in which one strand has at least one region complementary to a region on the other strand, and a single stranded molecule comprising a hairpin loop wherein at least one region of the hairpin loop is complementary to an opposing region of the hairpin loop. According to one embodiment, the siRNA comprises a double stranded RNA molecule wherein: (a) each strand of the siRNA molecule is independently about 15 to about 30 nucleotides in length; and (b) one strand of the siRNA molecule comprises a nucleotide sequence having a degree of complementarity to a Necl4 RNA sufficient to induce RNA interference. In another embodiment, the invention provides a method of down regulating Necl4 expression in a cell, comprising contacting the cell with an siRNA molecule of the invention, under conditions suitable for down regulating of Necl4 expression. In certain embodiments, the siRNA comprises at least one modification selected from the group consisting of: a 2′-sugar modification, a nucleic acid base modification, and a phosphate backbone modification. In another embodiments, the siRNA comprises an oligonucleotide selected from the group consisting of SEQ ID NO:55 and SEQ ID NO:56.

In another aspect, the invention provides a method of inhibiting Necl4 activity in a cell, the method comprising down regulating expression of Necl1 via RNA interference with an siRNA molecule specific for Necl1. According to one embodiment, the siRNA molecule specific for Necl1 comprises the oligonucleotide of SEQ ID NO:57. The invention further provides use of an siRNA molecule specific for Necl1 for the preparation of a composition for down regulating expression of Necl1 via RNA interference.

In another aspect, the invention provides antibodies specific for an isolated domain of Necl4 or a fragment thereof. In one embodiment, the domain comprises an amino acid sequence selected from the group consisting of: SEQ ID NO:2 (extracellular domain); SEQ ID NO:4 (cytoplasmic domain) and SEQ ID NO:5 (transmembrane domain). In one embodiment, the antibody is selected from the group consisting of: a polyclonal antibody, a monoclonal antibody, a humanized antibody, a chimeric antibody and an antibody fragment. In another embodiment, the antibody fragment is selected from the group consisting of: a single chain antibody, an Fab′, an F(ab)₂ and an F. In another embodiment, the antibody further comprises a detectable label selected from the group consisting of: radionuclides, ultrasound contrast agents, MRI contrast agents, dyes, fluorescent compounds and paramagnetic metals.

In another embodiment, the invention provides a method of diagnosing neurological damage in a subject, the method comprising administering to the subject the antibody of the invention; detecting the antibody by imaging techniques; and, optionally, evaluating the localization and/or amount of bound antibody and comparing said localization and/or amount with a localization and/or amount of bound antibody in a control healthy subject. In another embodiment of the method of diagnosing neurological damage in a subject, the antibody is selected from the group consisting of: single chain antibody, Fab, Fab′, F(ab′)₂, and F. In another embodiment, the antibody is conjugated to a diagnostic agent. In another embodiment, the diagnostic agent is selected from the group consisting of: radionuclides, ultrasound contrast agents, MRI contrast agents, dyes, fluorescent compounds and paramagnetic metals. In another embodiment, the radionuclide is useful in positron emission, and the radionuclide is selected from the group consisting of F-18, Mn-51, Mn-52m, Fe-52, Co-55, Cu-62, Cu-64, Ga-68, As-72, Br-75, Br-76, Rb-82m, Sr-83, Y-86, Zr-89, Tc-94m, In-110, I-120 and I-124. In another embodiment, the MRI contrast agent comprises a metal selected from the group consisting of gadolinium, manganese, iron, chromium, copper, cobalt, nickel, dysprosium, rhenium, europium, terbium, holmium and neodymium. In another embodiment, the radionuclide is useful in gamma-ray detection and is selected from the group consisting of: Cr-51, Co-57, Co-58, Fe-59, Cu-67, Ga-67, Se-75, Ru-97, Tc-99m, In-111, In-114m, I-123, I-125, I-131, Yb-169, Hg-197 and T1-201.

In another embodiment of the method of diagnosing neurological damage in a subject, the antibody is a humanized antibody. In one embodiment, the method further comprises administering to the subject a clearing agent and allowing the clearing agent to clear non-localized antibody. In another embodiment, the clearing agent is an anti-idiotypic antibody or antigen-binding antibody fragment. In another embodiment, the antibody conjugated to a diagnostic agent is administered by a method selected from the group consisting of: intravenous bolus, intravenous perfusion, intraarterial, intrapleural, intraperitoneal, intrathecal and subcutaneous. In another embodiment, the method is applied in conjunction with a method selected from: intraoperative probing, endoscopy and laparoscopy.

It is to be understood explicitly that the scope of the present invention encompasses homologs, analogs, mutants and derivatives of human Necl4 domains, including shorter and longer polypeptides and proteins, and polynucleotides encoding Necl 4, as well as polypeptide, protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these variants and modifications must preserve the capacities of a domain of human Necl4.

### BRIEF DESCRIPTION OF THE FIGURES

FIG. 1 shows multiple sequence alignment of Necl4 amino acid sequences from human, rat and mouse. Asterisks mark positions of amino acid identity among all compared sequences.

FIG. 2 relates to differential expression of Necls in the peripheral nervous system.

Panel a shows in situ hybridization analysis using Necl1, Necl2, Necl3 and Necl4 antisense probes against cross sections of DRGs connected to peripheral nerves prepared from neonate (P0) and 7 day-old (P7) rats. Dark and light arrowheads mark the location of the nerve and the DRG, respectively. Scale-bar: 200 mm.

Panel b shows RT-PCR analysis of RNA purified from dorsal root ganglia neurons (DRG); rat Schwann cells (RSC); brain and liver, using primers specific for Necl1, Necl2, Necl3, Necl4, gliomedin (GLDN) and actin.

The table summarizes expression of Necl1, Necl2, Necl3 and Necl4 in DRG neurons and Schwann cells, based on RT-PCR and in situ hybridization.

Panel c shows Northern blot analyses of RNA isolated from sciatic nerve at the indicated post-natal days using probes for Necl4 and Myelin P0 (MPZ). Ribosomal RNA markers are shown on the right.

FIG. 3 shows immunofluorescence labeling of isolated Schwann cells and DRG neurons with antibodies to Necl1 or Necl4. Schwann cell nuclei were labeled with Dapi (upper panels) and axons were labeled with an antibody to bIII Tubulin (TuJ; lower panels).

FIG. 4 indicates that Necl4 is the major Necl expressed by myelinating Schwann cells.

Panel A shows immunolabeling of transfected COS-7 cells expressing Necl1, Necl2, Necl3 or Necl4 with antibodies to Necl1 (upper row) and Necl4 (lower row). Insets show the expression of the different Necls as determined using an antibody to myc-tag (Necl2 and Necl3), binding of Necl4-Fc (Necl1), or Necl1-Fc (Necl4). Nuclei were labeled with Dapi (light spots) and the location of the cells shown in the inset is marked with an arrowhead.

Panel B shows Western blot analysis of extracts from rat brain membranes (RBM) and rat Schwann cells (RSC) upon immunoprecipitation (IP) with antibodies to Necl1, Necl4 (Ab), or preimmune serum control (PI), followed by immunoblotting with anti-Necl1 or anti-Necl4 antibodies as indicated. The panel on the right shows an immunoblot of sciatic nerve lysate using an antibody to Necl4 or the preimmune serum. Molecular weight markers (in kDa) are shown on the right.

FIG. 5 shows that axonal contact and myelination are associated with increased expression of Necl4 in Schwann cells.

Cultures of dissociated DRG neurons and Schwann cells were immunostained for Necl4, and bIII tubulin, MBP or MAG, 6, 10, or 17 days after plating as indicated; myelination was induced with ascorbic acid at day 10.

Panels a-d show increased labeling of Necl4 at contact sites between Schwann cells and axons (arrowheads), and insets in Panels c-d show higher magnification images of the arrowhead marked areas.

Panels e-j show that myelin basic protein (MBP) and myelin associated glycoprotein (MAG) positive myelinating Schwann cells (arrowheads) have intense Necl4-immunoreactivity. Scale bar: (a-d) 10 μm; (e-j) 30 μm.

FIG. 6 shows that Necl4 and Necl1 are localized along the internodes in rat sciatic nerve. The localization of Necl4 and Necl1 in myelinated nerves was determined using purified antibodies to these proteins in combination with antibodies to various axonal and glial markers.

Panels a-f show immunofluorescence double staining for Necl4 and: (a) neurofilament, (b) Na⁺ channels (Nach; to label nodes), (c) gliomedin (Gldn; to label nodes), (d) Kv1.2 (to label juxtaparanodes), (e) Caspr (to label paranodes), and (f) myelin-associated glycoprotein (MAG).

Panel f shows cross sections of adult rat sciatic nerves labeled for Necl4 and MAG. Arrowheads and asterisks mark the incisures and the nodes of Ranvier, respectively. Inset in Panel c shows a higher magnification of the nodal region. Insets in Panel e show a higher magnification of the paranodes labeled for Necl4 (upper), Caspr (middle) or the merged image (lower).

Panels h-k show teased fibers double immunolabeled for: Necl1 and Na⁺ channels (h); Necl1 and Caspr (i); Necl1 and Kv1.2 (j); and Necl1 and Necl4 (k). The location of the nodes and paranodes is marked with a horizontal line in h, or with dashed vertical lines in ij. Necl1 is present along the axonal internodes but is absent from the paranodes or the nodes of Ranvier. The Schmidt-Lanterman incisures, or their outermost ring (arrowhead) is labeled in Panel k.

Panels l-o show cross sections of adult rat sciatic nerves double labeled for: Necl4 and β-dystroglycan (DG; labels abaxonal Schwann cell membrane; Panel 1); Necl4 and neurofilament (NF; labels axons; Panel m); Necl1 and DG (Panel n), and Necl1 and NF (Panel o).

FIG. 7 shows immunolableling for Necl1 and Necl4 in sciatic nerve, using antibodies to Caspr (light areas) and Necl1 or Necl 4 (darker area). Staining antibody used is indicated on the left. Antibodies were used directly (none) or following pre-incubated with the GST-fusion protein that was the antigen used for immunization GST-fusion proteins used were that containing the intracellular (cytoplasmic) domain of Necl1 (GST-Necl1) or that containing the intracellular (cytoplasmic) domain of Necl4 (GST-Necl4). Scale bar: 10 μm.

FIG. 8 shows that Necl4 is expressed by myelinating, but not by non-myelinationg Schwann cells in sciatic nerve.

Panels a-d show immunofluorescence staining of teased adult rat sciatic nerve for Necl4 and: (a) GFAP, (b) L1, or (c-d) myelin-associated glycoprotein (MAG).

Panel e shows double labeling with antibodies to MAG and L1. Schwann cell nuclei were labeled with Dapi (light areas). Necl4 is expressed by myelinating Schwann cells (arrows) but is absent form non-myelinating Schwann cells (arrowheads), which are labeled with GFAP and L1. Scale bar: 10 μm.

FIG. 9 shows that Necl4 is the glial receptor for axonal Necl1.

Panel A (upper) shows a schematic diagram of fusion proteins containing a Necl extracellular domain fused to an immunoglobulin Fc region. Panel A (lower) shows Western blotting of soluble Necl-IgG 1-Fc fusion proteins with anti-human antibody. Molecular weight markers (in kDA) are shown on the right.

Panel B shows differential binding of Necl-Fc fusion proteins to DRG-neurons and Schwann cells. Soluble fusion proteins including the extracellular domain of Necl1 or Necl3 bound to Schwann cells, whereas no binding was detected with fusion proteins containing the extracellular domain of Nec2 or Necl4 (lower panel). In contrast, the extracellular domains of Nelc1-4 bound to neurites of cultured DRG-neurons (upper panel). Necl4-Fc robustly labeled the axons, Necl2-Fc showed moderate binding, and Necl1-Fc and Necl3-Fc showed weak binding.

FIG. 10 also shows that Necl4 is the glial receptor for axonal Necl1.

Panel A shows the ability of Necl-Fc fusion proteins including the extracellular domains of Necl1-Necl4 to bind to transfected COS-7 cells expressing Necl1 or Necl4.

Necl1-Fc binds to cells expressing Necl1-Necl4, while Necl4-Fc strongly bound to cells expressing Necl1 and Necl4 and weaker to cells expressing Necl2 or Necl3. The results indicate that Necl proteins display heterophilic binding (to each other) and homophilic binding (to themselves). Transfected cells were identified by immunostaining with rabbit antibodies to Necl or to Necl4 (insets) as indicated.

Panel B shows the effect of siRNA induced down regulation of Necl4 in Schwann cells on Necl1-Fc binding. Schwann cells were transfected with siRNA-oligonucleotides designed to knock down the expression of Necl4. Schwann cells transfected with Necl4-specific siRNA showed reduced binding of Necl1-Fc in proportion to the reduction of Necl4 expression, in contrast to cells transfected with a control siRNA. Minor amounts Necl4 in the cells transfected with Necl4-specific siRNA showed some binding to anti-Necl4 antiserum and to Necl1 (left panels, upper and lower respectively; indicated by arrowheads).

FIG. 11 shows a siRNA knock down experiment that is the reciprocal of that shown in FIG. 10B, and indicates that siRNA induced down regulation of neuronal Necl1 abolishes Necl4 binding. Binding of Necl4-Fc to DRG neurons were transfected with a Necl1-specific siRNA or control siRNA. Neurites were labeled with an antiserum to bIII-tubulin in the lower panels.

FIG. 12 shows that Necl1 and Necl4 co-cluster.

Panel A shows clustering of Necl1 in DRG-neurons by Necl4-Fc. Necl4-Fc was bound to Schwann cells, the excess washed away and allowed to cluster overnight at 37° C.; staining for Necl1 reveals co-localisation of Necl4-Fc and Necl1, indicating that Nec4-Fc can recruit Necl1 into its clusters and that Necl1 might be the neuronal receptor of Necl4.

Panel B shows clustering of Necl4 in Schwann cells by Necl1-Fc. Necl1-Fc was bound to Schwann cells, the excess washed away and allowed to cluster for four hours at room-temperature; staining for Necl4 reveals co-localisation of Necl1-Fc and Necl4 at the cell periphery at sites of cell-cell contact.

FIG. 13 shows that Necl4-Necl1 interaction mediates Ca²⁺-independent adhesion of Schwann cells. Schwann cells were allowed to adhere to plastic dishes pre-coated with Fc-fusion proteins containing the extracellular domain of Necl1 or Necl4, or the Fc region of human IgG (Fc) or laminin (lam) as controls, as indicated (substrate). Cell adhesion was not influenced by the presence or absence of Ca²⁺. The number of adhered cells is shown as a percentage of the cells that adhere to Necl1-Fc (y-axis). Schwann cells adhered to Necl1-Fc and to laminin, but not to Necl4-Fc. Blocking of the coated Necl1-Fc with Necl4-Fc (competitor) prior to addition of Schwann cells completely abolished their adhesion.

FIG. 14 shows that Necl-mediated axon-glia interaction is required for myelination.

Myelinated DRG-cultures were untreated (Panel a), or grown in the presence of Fc-fusion proteins containing the extracellular domain of Necl1 (Nel1-Fc; Panel b), Necl4 (Necl4-Fc; Panel c) or Zig 1 (Zig1-Fc; Panel d) for 10 days, and then immunostained for MBP. Panel e shows the number of MBP-positive segments as a percentage of that in the untreated cultures. Cultures grown in the presence of Necl-Fc's contained significantly (p<0.005) fewer myelin segments compared to untreated or control-treated (Zig1-Fc) cultures.

FIG. 15 shows analysis of myelinating cultures.

Panels a and b show representative electron microscopy images of 9 day old DRG and Schwann cells cultures grown in the presence of human Fc (h-Fc; Panel a) or Necl4-Fc (Panel b). Two additional examples are shown in the insets. While in the control Fc-treated cultures Schwann cell processes are circled 1.5 times around the axon, they fail to do so when the cultures were grown in the presence of Necl4-Fc (arrowheads). Scale bar: 20 nm.

Panel c shows the number of ensheathed and wrapped axons in each culture as a percentage of the total sites counted (hFc n=87; Necl4-Fc n=118).

FIG. 16 shows that addition of Necl4-Fc specifically inhibits myelination in myelinating cultures.

Dissociated DRG cultures were grown in the presence of Fc-fusion proteins containing the extracellular domain of Necl4 (Necl4-Fc; Panel b), MAG (MAG-Fc; Panel c), or human Fc (h-Fc: Panel a). Fresh fusion proteins were added every second day for an additional 10 days, then the cells were fixed, immunostained for MBP (green) and MBP-positive myelin sheaths were counted. Panel d shows the number of MBP-positive segments present in each condition as a percentage of that in the h-Fc-treated cultures. Cultures grown in the presence of Necl4-Fc contained significantly (p<0.0001) fewer myelin segments compared to the control h-Fc and MAG-Fc treated cultures. Note that in contrast to Necl4-Fc, MAG-Fc had no effect on myelination. Scale bar: 100 μm.

FIG. 17 shows that ectopic expression of Necl4 mutants modulates myelination.

Panel A is a schematic presentation of the different mutants expressed in Schwann cells and 3T3 cells. Necl4-FL is a full length Necl4; Necl4dCT lacks the entire cytoplasmic domain, including the 4.1 (square) and a PDZ (circle)-binding sequences. The transmembrane (TM) domain and the myc-tag (gray rectangle) are indicated. GFP-Necl4CT and GFP-NF155CT contain the cytoplasmic domain of Necl4 or neurofascin (NF) fused to green fluorescence protein (GFP), respectively.

Panel B shows Western blot analysis of infected Schwann cells and 3T3 cells infected with green fluorescence protein (GFP); Necl4FL (N4FL) or Necl4dCT N4dCT) using antibodies that recognize the cytoplasmic domain of Necl4 or the myc-tag sequence as indicated. Molecular weight markers (in kDa) are shown on the right.

Panel C shows immunolabeling of non-permeabilized 3T3 cells with anti-Necl4 or anti-myc tag antibodies, and binding by Necl1-Fc. Both Necl4-FL and Necl4-dCT reach the cell surface, and can bind Necl1.

Panel D shows expression of GFP-Necl4CT and GFP-NF155CT in 3T3 cells, as analyzed by immunolabeling with antibodies to Necl4 (αNecl4CT) or neurofascin (αNF155CT) as indicated. Insets show the fluorescence signal of the GFP.

Panel E shows the effect of Necl4 expression on myelination. Schwann cells infected with retroviruses that direct the expression of GFP or the indicated Necl4 constructs were allowed to myelinate sensory DRG axons and the extent of myelination was determined by immunolabeling with an antibody to MBP. Quantification of the results obtained from several experiments is shown as percentage in the lower left of each panel.

Panel F shows that ectopic expression of Necl4-FL (FL), as well as GFP-Necl4ct (N4CT) in Schwann cells inhibited myelination by 60-65% (p<0.001), while the cytoplasmic domain of neurofascin (NFCT) had no significant effect as compared to cultures infected by the vector alone (GFP). Expression of Necl4-dCT (dCT; Necl4 lacking the cytoplasmic domain) resulted in a marked increase (600%; p<0.001) in the number of myelinating segments.

FIG. 18 shows that Necl4-Fc inhibits remyelination in vivo.

Demyelination of adult rat sciatic nerves was induced by lysolecithin followed by intraneural injections of human Fc (Panel a) or Necl4-Fc (Panel b). Eleven days post-injection, nerves were collected and longitudinal sections of the injected region were immunostained for MBP and Na⁺ channels. The border between demyelinated and unaffected region is marked by a dotted line. Insets depict higher magnification images of selected nodes from remyelinated regions, many of which have binary clusters of Na⁺ channels. Circles mark the locations of representative regions shown in Panels c-f: unaffected nerve regions (Panel c), remyelinated (Panel d) or inhibited (Panel e) areas in nerves injected with Necl4-Fc, as well as in remyelinated regions of nerves injected with hFc (Panel f). Regions of inhibited remyelination were only detected in the Necl4-Fc and not in the hFc control-injected nerves. Panel g shows the number of Na⁺ channel clusters in the regions defined in c-f as an average number (mean±SEM) per field of view (FOV a minimum of 10 FOV were counted for each region). Norm, normal; Re, remyelinated. Scale bar: (a-b) 100 μm; (c-f) 20 μm.

### DETAILED DESCRIPTION OF TILE INVENTION

### Definitions

The terms “Necl”, “Necl molecule” and “Necl protein molecule” are used herein interchangeably to refer to any one or more of the members of the Nectin-like (Necl) protein molecules, including Necl1 (also named IgSF4B/SynCAM3/Tsl11); Necl2 (also named IgSF4A/SynCAM1/Tslc1/RA175/SgIGSF); Necl3 (also named IgSF4D/SynCAM2), and Necl4 (also named IgSF4C/SynCAM4/TSLL2).

Accordingly, a Necl molecule comprises an amino acid sequence corresponding to that determined for the respective Necl member in any species including human, mouse, rat, dog and chimpanzee, and includes polypeptide and peptide fragments, domains, subregions, chimeric proteins, fusion proteins, mutants, analogs, homologs, and peptidomimetics.

The term “Necl4” as used herein refers to a Necl4 protein molecule, and includes but is not limited to the full-length protein, fragments and individual domains of Necl4.

The term “Necl1” as used herein refers to a Necl1 protein molecule, and includes but is not limited to the full-length protein, fragments and individual domains of Necl1.

The term “Necl2” as used herein refers to a Necl2 protein molecule, includes but is not limited to the full-length protein, fragments and individual domains of Necl2.

The term “Necl3” as used herein refers to a Necl3 protein molecule, includes but is not limited to the full-length protein, fragments and individual domains of Necl3.

The term “domain” as used herein refers to a structural/topographical or functional subunit of a full length or complete protein. A domain may be found within the context of the full length or complete protein, or may be separate therefrom, as in an isolated domain. Domains corresponding to structural/topographical subunits, include for example, a cytoplasmic domain, an extracellular domain and a transmembrane domain. Domains corresponding to functional subunits, include for example, a receptor binding domain. Accordingly, a “domain of Necl4” as used herein can refer to a structural/topographical subunit, for example, the cytoplasmic domain (Necl4-CT), the extracellular domain (Necl4-ECD) or the transmembrane domain (Necl4-TM), and/or to a functional subunit, for example, a myelination enhancing domain.

The term “isolated domain of human Necl4” refers to a domain of human Necl4 which is separate from and not in the context of the full length Necl4 protein.

The term “a heterologous human Necl” as used herein means a human Necl member other than Necl4, for example, Necl1, Necl2 or Necl3.

The term “domain of a heterologous human Necl” as used herein means a domain from a human Ned member other than Necl4. For example, a “domain of a heterologous human Necl” can be a domain from any of human Necl1, Necl2 and Necl3, but not from human Necl4.

The term “exogenous domain of Necl4” as used herein means that the genetic material encoding the domain of Necl4 is artificially introduced to the cell, and can be different to that normally expressed by the cell.

As used herein, the term “a polypeptide comprising an isolated domain of Necl4” expressly excludes a polypeptide corresponding to the full length amino acid sequence of human Necl4 as set forth in SEQ ID NO:1.

The term “derivative” as used herein, refers to protein or polypeptide variants including fragments, chimeric proteins, fusion proteins, mutants, homologs, and peptidomimetics.

The term “therapeutically effective amount” as used herein means an amount of an active ingredient, such as a domain of Necl4, which is sufficient to treat, alleviate, ameliorate or prevent neurological damage.

The term “neurological damage” as used herein, refers to any defect occurring in either the central or peripheral nervous system which is sustained a result of physical injury, disease or defective development. Neurological damage can variously occur in nervous tissue and the tissue that supports it including, but not limited to neuronal cells, Schwann cells, stellate cells, neuroglial cells, granule cells, ganglia cells, grey matter, white matter, myelin, neurolimma, axons, dendrites, motor neurons, fibrils and fibular processes. Neurological damage can be associated with demyelination or excessive myelination.

The term “neural cell” as used herein means any type of cell associated with the nervous system, including neuronal cells, Schwann cells, stellate cells, neuroglial cells, granule cells, ganglia cells and the like.

The term “demyelination” as used herein means loss or destruction of myelin in the central or peripheral nervous system.

The terms “polynucleotide sequence” and “polynucleotide construct” are used herein interchangeably to refer to a polymer of nucleotides, such as deoxyribonucleotides, ribonucleotides, or modified forms thereof in the form of an individual fragment or as a component of a larger construct, in a single strand or in a double strand form. The polynucleotides to be used in the invention include sense and antisense polynucleotide sequences of DNA or RNA as appropriate to the goals of the therapy practiced according to the invention. The DNA or RNA molecules may be complementary DNA (cDNA), genomic DNA, synthesized DNA or a hybrid thereof or an RNA molecule such as mRNA. Accordingly, as used herein, the terms “DNA construct”, “gene construct” and “polynucleotide” are meant to refer to both DNA and RNA molecules.

As used herein, the terms “recombinant protein” and “recombinant polypeptide” refer interchangeably to a protein produced using cells that do not have, in their native state, an endogenous copy of the DNA able to express the protein. The cells produce the recombinant protein because they have been genetically altered by the introduction of the appropriate isolated nucleic acid sequence, for example as a component of an expression vector.

The terms “recombinant fusion protein” and “fusion protein” are used herein interchangeably to refer to a protein produced by recombinant technology which comprises segments i.e. amino acid sequences, from heterologous sources, such as different proteins or different organisms. The segments are joined either directly or indirectly to each other via peptide bonds. By indirect joining it is meant that an intervening amino acid sequence, such as a peptide linker is juxtaposed between segments forming the fusion protein.

The term “siRNA” as used herein means a small interfering RNA (siRNA) that can act as a mediator of RNA interference (RNAi). RNAi is a process by which a particular cellular RNA sequence is targeted and destroyed upon activation of cellular enzymes, as a result of complementarity between the siRNA and the particular RNA sequence, resulting in its degradation.

As used herein, siRNA molecules include double stranded RNA (dsRNA) molecules comprising two separate RNA strands in which one strand has at least one region complementary to a region on the other strand, and single stranded RNA molecules comprising a hairpin loop i.e. comprising a double stranded RNA (dsRNA) region, wherein one strand of the double stranded region has at least one region complementary to a region on the other strand of the double stranded region.

The terms “down-regulate” and “inhibit” are used herein interchangeably to mean that the expression of a gene, or the level of RNAs encoding one or more corresponding protein subunits, or the activity of one or more corresponding protein subunits, such as Necl4 or a domain thereof, is reduced to below that observed in the absence of an siRNA molecule of the invention.

The present invention provides a polypeptide comprising an isolated domain of Necl4, or a fragment, analog or derivative thereof. The Necl4 is preferably human Necl4.

The nucleotide sequence of the gene encoding human Necl4 and the deduced Necl4 amino acid sequence are disclosed in Fukuhara et al (2001) Oncogene 20:5401-5407, and under GenBank Accession No. NM 145296. Human Necl4 is a protein of 388 amino acids which is composed of an extracellular domain spanning positions 1-323, including a signal sequence at positions 1-24 and three immmunoglobulin-like loops at positions 29-121, 136-221 and 236-298, respectively; a transmembrane domain spanning positions 324-346 and a cytoplasmic domain spanning positions 347-388, including a 4.1 protein-binding motif at positions 344-362 and a type II PDZ domain-binding sequence at positions 385-388.

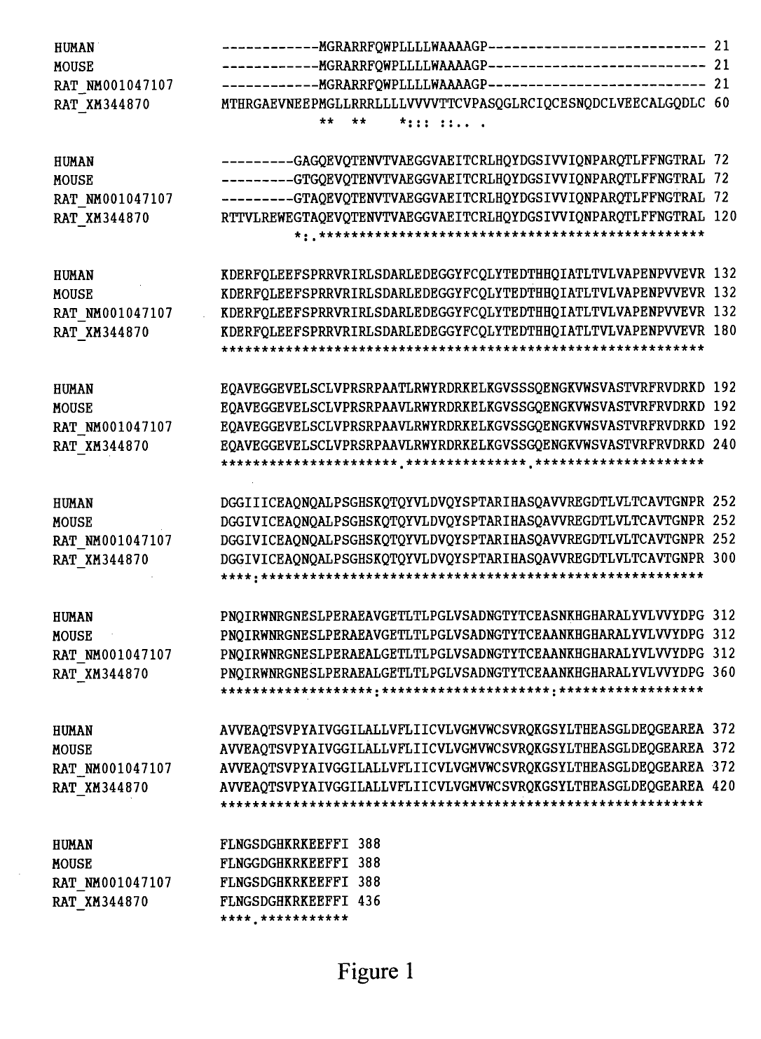

The full length nucleotide sequences of genes encoding rat Necl4 and the corresponding deduced Necl4 amino acid sequences are disclosed under GenBank Accession No. XM_{—}344870 and under GenBank Accession No. NM_{—}001047107. As shown in FIG. 1, these deduced rat Necl4 protein amino acid sequences are highly homologous, differing mainly in the N-terminal region comprising the signal sequence. The protein encoded by the gene of NM_{—}001047107 has 388 amino acids, and is composed of an extracellular domain spanning positions 1-323, including a signal sequence at positions 1-24 and three immmunoglobulin-like loops at positions 29-121, 136-221 and 236-298, respectively; a transmembrane domain spanning positions 324-346 and a cytoplasmic domain spanning positions 347-388, including a 4.1 protein-binding motif at positions 344-362 and a type II PDZ domain-binding sequence at positions 385-388. The protein encoded by the gene of XM_{—}344870 has 436 amino acids, and is composed of an extracellular domain spanning positions 1-371, including a signal sequence at positions 1-36 and three immmunoglobulin-like loops at positions 87-154, 186-249 and 286-341, respectively; a transmembrane domain spanning positions 372-394 and a cytoplasmic domain spanning positions 395-436, including a 4.1 protein-binding motif at positions 392-410 and a type II PDZ domain-binding sequence at positions 433-436.

The nucleotide sequence of the gene encoding mouse Necl4 and the deduced Necl4 amino acid sequence are disclosed in Fukami et al (2003) Gene 323:11-18, and under GenBank Accession No. NM_{—}153112. Mouse Necl4 is a protein of 388 amino acids which is composed of an extracellular domain spanning positions 1-323, including a signal sequence at positions 1-24 and three immmunoglobulin-like loops at positions 29-121, 136-221 and 236-298, respectively; a transmembrane domain spanning positions 324-346 and a cytoplasmic domain spanning positions 347-388, including a 4.1 protein-binding motif at positions 344-362 and a type II PDZ domain-binding sequence at positions 385-388. The structure of mouse Necl4 is highly homologous to that of human Necl4 and differs only at three amino acid positions.

As disclosed herein, Necl4 is the major Necl present on glial cells in the peripheral nervous system and the central nervous system, and its expression is dramatically increased during periods of active myelination. Furthermore, Necl4 interacts with Necl1, found on axons of neuronal cells, and together these molecules mediate glial-axonal contact required for myelination. More specifically, individual domains of Necl4 exhibit distinct functional activities. As disclosed herein, the extracellular domain of Necl4 has the ability to bind to axons, and in particular, to the extracellular domain of axonal Necl1. Furthermore, the cytoplasmic domain and the extracellular domain of Necl4, each individually influence the extent of myelination in neuronal cells.

Hence, without wishing to be bound by any theory or mechanism of action, Necl4 domains have an important role in the nerve building process. Accordingly, it is advantageous to provide a polypeptide comprising an isolated domain of Necl4 and a pharmaceutical composition comprising a polypeptide comprising an isolated domain of Necl4, which can be used for the treatment of neurological disorders associated with aberrant myelination.

A Necl4 domain according to the invention includes one or more of the amino acid sequences set forth in SEQ ID NO:2; SEQ ID NO:3; SEQ ID NO:4; SEQ ID NO:5; SEQ ID NO:6; SEQ ID NO:7, SEQ ID NO:8; SEQ ID NO:9, SEQ ID NO:10; SEQ ID NO:11; SEQ ID NO:12; SEQ ID NO:13; SEQ ID NO:98 and SEQ ID NO:99.

A Necl4 extracellular domain may or may not include the native Necl4 protein signal sequence at the N-terminus. A Necl4 extracellular domain including a Necl4 signal sequence encompasses that from human (SEQ ID NO:2), rat (SEQ ID NOS:6 and 98) or mouse (SEQ ID NO:2. A Necl4 extracellular domain lacking the Necl4 signal sequence encompasses that from human (SEQ ID NO:3), rat (SEQ ID NOS:7 and 99) and mouse (SEQ ID NO:11).

A Necl4 signal sequence encompasses that from human Necl4 (SEQ ID NO:14), rat Necl4 (SEQ ID NOS:15 and 102) and mouse Necl4 (SEQ ID NO:16. A Necl4 cytoplasmic domain encompasses that from human (SEQ ID NO:4), rat (SEQ ID NO:8) and mouse (SEQ ID NO:12).

A Necl4 transmembrane domain encompasses that from human (SEQ ID NO:5), rat (SEQ ID NO:9) and mouse (SEQ ID NO:13).

The polypeptide of the invention may contain more than one Necl4 domain, but it preferably and generally will not contain a complete Necl4 protein of any particular species. In one embodiment, the domain of Necl4 is selected from the group consisting of: (i) the cytoplasmic domain wherein the polypeptide lacks at least one domain of Necl4 selected from the group consisting of: the extracellular domain and the transmembrane domain; and (ii) the extracellular domain, wherein the polypeptide lacks at least one domain of Necl4 selected from the group consisting of: the cytoplasmic domain and the transmembrane domain. In specific embodiments, the polypeptide comprises the extracellular domain of human Necl4 as set forth in SEQ ID NO:2 or SEQ ID NO:3; or the cytoplasmic domain of human Necl4 as set forth in SEQ ID NO:4; or the transmembrane domain of human Necl4, as set forth in SEQ ID NO:5, or fragments, analogs and derivatives thereof.

According to the invention, a polypeptide comprising an isolated Necl4 domain can include or lack the native Necl4 signal sequence. Since the signal sequence is required for cell surface expression but not for biological activity, it can be advantageous to provide an isolated Necl4 domain lacking the signal sequence. An isolated Necl4 domain lacking the signal sequence can be produced by methods known in the art. Alternately, an oligonucleotide encoding the Necl4 domain or the polypeptide may be engineered to include a signal sequence upstream of the polypeptide coding sequence in order to facilitate its expression on a cell surface. The signal sequence may be that of the native protein i.e. the signal sequence of human Necl4 (SEQ ID NO:14), the signal sequence of rat Necl4 (SEQ ID NOS:15 or 102), the signal sequence of mouse Necl4 (SEQ ID NO:16), or that of a heterologous protein. A non-limiting example of a signal sequence of a heterologous protein is the signal sequence of immunoglobulin kappa chain (SEQ ID NO:48). A non-limiting example of a polypeptide according to the invention which includes a heterologous signal sequence is provided by SEQ ID NO:47 which comprises the signal sequence of immunoglobulin kappa chain and the extracellular and transmembrane domains of rat Necl4.

A polypeptide comprising an isolated Necl4 domain also encompasses shorter and longer polypeptides, fragments, derivatives, and analogs of SEQ ID NOS:2-5. Derivatives of the extracellular domain of human Necl4 include for example, SEQ ID NO:6; SEQ ID NO:7, SEQ ID NO:10, SEQ ID NO:11, SEQ ID NO:98 and SEQ ID NO:99. Derivatives include chimeric proteins, fusion proteins, mutants, homologs, and peptidomimetics. A derivative may fall into one or more of the aforementioned categories. For example, a Necl4 domain from a particular species e.g. rat, may be mutated to have the amino acid sequence of its homolog from a different species e.g. human. Accordingly, such a mutated Necl4 domain is both a mutant and a homolog. Furthermore, a chimeric Necl4 domain may comprise segments from different species e.g. rat and mouse, and may further be a fusion protein when fused to a heterologous sequence such as an immunoglobulin Fc region.

A polypeptide comprising an isolated domain of Necl4 can be a recombinant product prepared using recombinant DNA methodology and expression in a suitable host cell, as is known in the art (see for example Sambrook et al., (2001) Molecular Cloning, Cold Spring Harbor Laboratory Press, Cold Spring Harbor, N.Y.). Nucleotide sequences encoding specific Necl4 domains may be conveniently prepared, for example by polymerase chain reaction using appropriate oligonucleotide primers corresponding to the 5′ and 3′ regions of the domain required for isolation, and a full length coding Necl4 sequence as template. The source of the full length coding Necl4 sequence may be for example, DNA extracted from neural cells or a plasmid vector containing a cloned Necl4 gene.

The nucleotide sequence encoding the Necl4 domain may be used on its own to generate the polypeptide, or it may be fused with coding sequence(s) of a heterologous protein or fragment or domain thereof in order to direct expression of a fusion protein or chimeric protein. The heterologous protein may be a different Ned, for example a domain of Necl1, or a protein which is not a Ned. In particular, the heterologous protein may be an immunoglobulin, a marker protein, a protein associated with neural cells, a signal sequence or a particular fragment of any of the aforementioned. Immunoglobulin fragments include, without limitation Fc, Fab, scFv, dsFv, V_{L} and V_{H}. In some cases, it may be desirable to select a coding sequence of an immunoglobulin or immunoglobulin fragment which has specificity for a protein associated with neural cells. In other cases, it may be desired to select a coding sequence for an immunoglobulin Fc region. An example of a polynucleotide encoding an Fc-Necl4 extracellular domain fusion protein is provided by SEQ ID NO:83. In other cases, the coding sequence of the heterologous protein encodes a protein associated with neural cells. A protein associated with neural cells includes without limitation MBP (myelin basic protein); MAG (myelin associated glycoprotein); Caspr; GFAP (glial fibrillary acidic protein); L1; contactin; neurofascin; TAG-1; Nr-CAM (neuron-glia-related cell-adhesion molecule) and gliomedin.

The DNA sequence encoding the polypeptide is cloned into an expression vector or plasmid, selected to direct the synthesis of the polypeptide upon transformation into a suitable host cell, including E. coli, other bacterial hosts, yeasts and various higher eucaryotic cells such as for example, COS, 293T, CHO, Schwann cell and HeLa cell lines.

The recombinant DNA sequence encoding the polypeptide will be operably linked to appropriate expression control sequences for each host. For E. coli this includes a promoter such as the T7, trp, or lambda promoters, a ribosome binding site and preferably a transcription termination signal. For eucaryotic cells, the control sequences generally include a promoter and an enhancer e.g. derived from immunoglobulin genes, SV40, cytomegalovirus, etc., and a polyadenylation sequence, and may include splice donor and acceptor sequences. The plasmids can be transferred into the chosen host cell by well-known methods such as calcium chloride transformation for E. coli and calcium phosphate treatment or electroporation for eucaryotic cells. Cells transformed by the plasmids can be selected by resistance to antibiotics conferred by genes contained on the plasmids, such as the ampicillin resistance gene.

Once expressed, the recombinant polypeptide can be purified according to standard procedures of the art, including ammonium sulfate precipitation, affinity columns, column chromatography, gel electrophoresis and the like, as is known in the art. Substantially pure compositions of at least about 90 to 95% homogeneity are preferred, and 98 to 99% or more homogeneity are most preferred for pharmaceutical uses.

Alternately, an isolated domain of Necl4 may be produced as a cleavage product or fragment of a native polypeptide isolated from tissue of an animal species. Alternately, an isolated polypeptide comprising a domain of Necl4 can be chemically synthesized.

A Necl domain, and particularly a Necl4 domain, or a fragment thereof, can provide a specific polypeptide region or subunit which exhibits a particular biological activity. A fragment of a Necl domain which exhibits a particular biological activity may also be termed an active fragment. As disclosed herein, the cytoplasmic domain and the extracellular domain of rat Necl4 exhibit distinct, and possibly opposing, biological activities with respect to myelination of neuronal cells. Accordingly, it is advantageous to provide an isolated Necl4 domain, or an active fragment thereof, comprising either the cytoplasmic domain and/or the extracellular domain, as the active ingredient of a pharmaceutical composition. The fragment selected depends on the neurological condition to be treated and the corresponding therapeutic effect desired. For example, a neurological condition associated with demyelination would advantageously be treated with a particular Necl4 domain having activity in enhancing myelination, while a neurological condition associated with excessive myelination would advantageously be treated with a Necl4 domain having activity in inhibiting myelination.

In addition, Necl1 is disclosed herein to be the binding partner of Necl4, with both proteins acting in concert to promote myelination. Accordingly, it may be advantageous to provide specific domains of both Necl4 and Necl1 in the same fusion protein, and/or in the same pharmaceutical composition when the combined activities of those domains is required. Similarly, it may be advantageous to provide polynucleotide sequences encoding domains of both Necl4 and Necl1 in the same expression vector, host cell or pharmaceutical composition for gene therapy of aberrant neurological conditions which can be ameliorated by the combined expression and activities of Necl4 and Necl1 domains.

The active fragment may furthermore correspond to a portion, and preferably the minimal portion, of a particular Necl domain which retains its biological activity. The amino acid sequence and nucleotide sequence encoding such an active fragment may be determined by one skilled in the art. For example, progressively shorter fragments of a Necl4 domain can be produced upon construction and expression of progressively shorter mutants of a nucleotide sequence encoding that domain. The mutants can be expressed in a cell culture, such as a Schwann cell culture, and thereafter analyzed for biological activity in a myelination assay as disclosed herein. The shortest such active fragment retaining the desired biological activity may advantageously be selected as the active ingredient of a pharmaceutical composition. Alternately, such an active fragment can form the basis of a peptidomimetic, or it can be used as the basis of a screening procedure for a small chemical entity which exerts the biological activity of the Necl4 domain.

The Necl fragment may furthermore be mutated to include one or more amino acid substitution, or non-natural amino acid with respect to the native sequence. Preferably such mutants retain the biological activity of the Necl native fragment or domain.

The terms “mutant” and “analog” are used interchangeably herein to describe altered proteins e.g. of Necl4 or fragments thereof, and includes shorter and longer polypeptides and/or proteins with one or more amino acid substitution, non-natural amino acid or chemical modification. It is to be understood that mutant proteins intended for therapeutic applications will exhibit a desired biological activity, e.g. the ability to interact with Necl1, whereas other mutants may be devoid of that biological activity and can be useful for demonstrating or delinating domain regions having biological activity. A non-limiting example of a Necl4 mutant is SEQ ID NO:47, a polypeptide consisting of rat Necl4 extracellular and transmembrane domains in which the native rat Necl4 signal sequence has been replaced by the Ig kappa chain signal sequence and a myc tag.

The term “amino acid substitution” means that a functionally equivalent amino acid residue is substituted for a residue within the sequence resulting in a silent change. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Additionally, a non-conservative substitution may be made in an amino acid that does not contribute to the biological activity, e.g. Necl1 binding.

As used herein, a chimeric protein means a protein sequence comprising sequences of more than one different Necl member e.g. Necl1-4, and/or Necl sequences of distinct animal species. Thus for example, a chimeric Necl protein can comprise a Necl4 sequence (either the full length sequence or a fragment thereof) continuous with a Necl1 sequence (either the full length sequence or a fragment thereof). The two sequences can be from the same or different animal species. Alternately, a chimeric Necl protein can comprise portions of the same Necl member derived from distinct animal species. Thus for example, a chimeric Necl4 protein can comprise an N-terminal fragment of Necl4 from mouse continuous with a C-terminal fragment of Necl4 from rat. A non-limiting example of a chimeric Necl4 protein is SEQ ID NO:46, comprising amino acids 28 to 284 of mouse Necl4, continuous with amino acids 333 to 436 of rat Necl4 (XM_{—}344870). SEQ ID NO:46 further comprises an Ig kappa chain and a myc tag at the N-terminal region, meaning that it is also a fusion protein. SEQ ID NO:46 may be expressed by the polynucleotide sequence of SEQ ID NO:78, which is contained within expression plasmid pC3.1-Necl4 (SEQ ID NO:79), disclosed herein. An additional chimeric Necl4 protein which further corresponds to isolated domains of Necl4, is SEQ ID NO:47, which is a mutated form of SEQ ID NO:46 in that it lacks the cytoplasmic domain of rat Necl4 i.e. amino acids 394 to 436. SEQ ID NO:47 may be expressed by the polynucleotide sequence of SEQ ID NO:94, which is contained within expression plasmid pMX-SSmyc-Necl4dCT (SEQ ID NO:95), disclosed herein. As used herein, a fusion protein means a protein comprising segments from different i.e. heterologous proteins wherein the segments are linked via peptide bonds. The linkage may be direct i.e. contiguous, or indirect, meaning that the segments are joined via an intervening peptide linker or spacer or other amino acid sequence. Accordingly, a fusion protein may consist of a first segment which is a Necl domain (or a fragment thereof) linked to a second segment of a heterologous protein which may or may not be a Necl. The heterologous protein may be for example an immunoglobulin, a marker protein, a protein associated with neural cells, a signal sequence, a heterologous Necl, or a particular fragment of any of the aforementioned. Immunoglobulin fragments include, without limitation Fc, Fab, scFv, dsFv, V_{L} and V_{H}. The immunoglobulin or immunoglobulin fragment may be selected to have specificity for a protein associated with neural cells, in which case the immunoglobulin or immunoglobulin fragment comprises antigen binding region(s). In other cases, it may be useful to produce a fusion protein comprising the immunoglobulin Fc region as the heterologous protein segment. For example, an Fc-human Necl4 extracellular domain fusion protein may be produced using SEQ ID NO:44 directly fused to SEQ ID NO:2 to produce Fc-human Necl4 extracellular domain. A marker protein may be a visual reporter protein such as Green Fluorescent Protein (GFP), or a genetic tag, such as myc.

In other cases, the heterologous protein may be a protein associated with neural cells. A protein associated with neural cells includes without limitation MBP (myelin basic protein); MAG (myelin associated glycoprotein); Caspr; GFAP (glial fibrillary acidic protein); L1; contactin; neurofascin; TAG-1; Nr-CAM (neuron-glia-related cell-adhesion molecule) and gliomedin.

A non-limiting example of a Necl4 fusion protein is provided by the product of expression plasmid pMX-GFP-Necl4ct (SEQ ID NO:91), comprising the nucleotide sequence SEQ ID NO:90, which encodes the cytoplasmic domain of rat Necl4 (SEQ ID NO:8) in fusion with EGFP (SEQ ID NO:45). Other Necl4 fusion proteins include, without limitation SEQ ID NO:46 and SEQ ID NO:47, described hereinbefore.

As used herein, the term “homologs” refers to distinct polypeptide sequences which exhibit significant amino acid homology. The degree of homology between or among two or more amino acid sequences is assessed by the degree of amino acid identity and similarity between them, as determined by computerized sequence alignment, for example BLAST analysis (Altschul et al., 1990). Among homologous polypeptides, the homology may occur along the full length of the polypeptides, or it may be limited to specific regions, for example immunoglobulin-like domains. The identification of homologous sequences within polypeptides from different animal species indicates that the polypeptides may be phylogenetically related, and/or may have similar function and/or mechanism of action. As used herein, “homologs” have at least 80%, 81%, 82%, 83%, 84%, 85%, 86%, 87%, 88%, 89%, 90%, 91%, 92%, 93%, 94%, 95%, 96%, 97%, 98%, 99% or 100% identity along their length.

As used herein, “significant amino acid homology” means at least about 80% identity between or among two or more amino acid sequences. More preferably, “significant homology” means at least about 85% identity between or among two or more amino acid sequences. More preferably, “significant homology” means at least about 90%, 91%, 92%, 93%, 94%, 95%, 96%, 97%, 98%, 99% or 100% identity between or among two or more amino acid sequences.

Examples of Necl homologs having significant homology are Necl4 molecules from human, rat and mouse, as shown in FIG. 1. The regions of significant homology include the extracellular domains, the cytoplasmic domains and the transmembrane domains. For example, the cytoplasmic domains of Necl4 from human, rat and mouse (corresponding respectively to SEQ ID NOS.:4, 8 and 12), are homologous Necl4 domains, with the human and rat domains exhibiting 100% identity.

As used herein, a peptidomimetic means a molecule that mimics the biological activity of a particular peptide or polypeptide, for example a Necl4 molecule, but is not completely peptidic in nature. Whether completely or partially non-peptide, peptidomimetics according to this invention provide a spatial arrangement of chemical moieties that closely resembles the three-dimensional arrangement of groups in the Necl molecule on which the peptidomimetic is based. As a result of this similar active-site geometry, the peptidomimetic has effects on biological systems which are similar to the biological activity of the Ned molecule.

According to the invention disclosed herein, an isolated Necl domain can further comprise a chemical modification. Such modifications include but are not limited to glycosylation, pegylation, oxidation, permanent phosphorylation, reduction, myristylation, sulfation, acylation, acetylation, ADP-ribosylation, amidation, hydroxylation, iodination, methylation, and derivatization by blocking groups.

As disclosed herein, Necl4 exhibits heterophilic binding to each of Necl1, Necl2 and Necl3. As disclosed herein at least some of these binding interactions mediate axial-glial interaction required for myelination in the PNS and/or CNS. Without wishing to be bound by any theory, heterophilic binding among Necl molecules may be part of the nerve building process. Accordingly, it is advantageous to provide a pharmaceutical composition comprising an isolated domain of Necl4, and further comprising an isolated domain of a heterologous Necl. Such a pharmaceutical composition can be used for the treatment of neurological disorders associated with aberrant myelination.

Accordingly, the invention provides a pharmaceutical composition comprising as an active ingredient a polypeptide comprising an isolated domain of human Necl4, or a fragment, analog or derivative thereof, and further comprising at least one isolated domain of a heterologous human Necl, or a fragment, analog or derivative thereof. A heterologous human Necl includes human Necl1, Necl2 and Necl3, but not human Necl4. A domain of a heterologous human Necl means that the source of the domain is a human Necl member which is not human Necl4. Accordingly, an “isolated domain of a heterologous human Necl” can be an isolated domain from any of human Necl1, Necl2 and Necl3, but not from human Necl4. Furthermore, the domain can be a structural domain, such as a cytoplasmic domain, an extracellular domain or a transmembrane domain, and/or a functional domain, such as a myelination enhancing domain.

Accordingly, the isolated domain of a heterologous human Ned may be selected from the group consisting of: the extracellular domain of human Necl1 as set forth in SEQ ID NO:17; the cytoplasmic domain of human Necl1 as set forth in SEQ ID NO:18; the transmembrane domain of human Necl1 as set forth in SEQ ID NO:19; the extracellular domain of human Necl2 as set forth in SEQ ID NO:20; the cytoplasmic domain of human Necl2 as set forth in SEQ ID NO:21; the transmembrane domain of human Necl2 as set forth in SEQ ID NO:22; the extracellular domain of human Necl3 as set forth in SEQ ID NO:23; the cytoplasmic domain of human Necl3 as set forth in SEQ ID NO:24 and the transmembrane domain of human Necl3 as set forth in SEQ ID NO:25.

A derivative of the domain of a heterologous human Necl may be selected from the group consisting of SEQ ID NOS.:26-43. SEQ ID NOS.:26-28 correspond respectively to the extracellular domain, the cytoplasmic domain and the transmembrane domain of rat Necl1. SEQ ID NOS.:29-31 correspond respectively to the extracellular domain, the cytoplasmic domain and the transmembrane domain of rat Necl2. SEQ ID NOS.:32-34 correspond respectively to the extracellular domain, the cytoplasmic domain and the transmembrane domain of rat Necl3. SEQ ID NOS.:35-37 correspond respectively to the extracellular domain, the cytoplasmic domain and the transmembrane domain of mouse Necl1. SEQ ID NOS.:38-40 correspond respectively to the extracellular domain, the cytoplasmic domain and the transmembrane domain of mouse Necl2. SEQ ID NOS.:41-43 correspond respectively to the extracellular domain, the cytoplasmic domain and the transmembrane domain of mouse Necl3.

The term “Necl domain” as used herein refers to one or more Necl protein domains, and includes subregions, subunits, fragments, derivatives, chimeric proteins, fusion proteins, mutants, analogs, homologs, and peptidomimetics. An isolated Necl domain may be a recombinant expression product encoded by an isolated or cloned gene, DNA or RNA fragment or it can be a cleavage product or fragment of a native polypeptide isolated from tissue of an animal species. Alternately an isolated Necl domain may be chemically synthesized. Fragments, derivatives, chimeric proteins, fusion proteins, mutants, analogs, homologs, and peptidomimetics are as hereinbefore defined.

### Pharmaceutical Compositions

The present invention provides a pharmaceutical composition comprising as an active ingredient, a therapeutically effective amount of a polypeptide comprising an isolated domain of Necl4 or a fragment, analog or derivative thereof; or a therapeutically effective amount of an expression vector encoding a domain of Necl4, or a fragment, analog or derivative thereof; or a therapeutically effective amount of an siRNA capable of down regulating Necl4 expression. The Necl4 is preferably human Necl4. The active ingredient may be combined with a pharmaceutically acceptable carrier, as is known in the art.

As used herein, the term “pharmaceutically acceptable carrier” includes any and all solvents, diluents, dispersion media, coatings, surfactants, detergents, antioxidants, adjuvants, preservatives (e.g., antibacterial agents, antifungal agents), isotonic agents, bulking agents, absorption delaying agents, salts, drug stabilizers, gels, binders, excipients, disintegration agents, lubricants, sweetening agents, flavoring agents, dyes, similar materials and combinations thereof, as is known to one of ordinary skill in the art (see for example, Remington's Pharmaceutical Sciences, 18th Ed. Mack Printing Company, 1990). Any pharmaceutical carrier is contemplated, except for those incompatible with the active ingredient, and/or those likely to produce a significant adverse, allergic or other untoward reaction when administered to a human in the amount(s) present in the pharmaceutical composition.

The choice of carriers will depend on the physico-chemical properties of the active ingredient and the route of administration contemplated for the pharmaceutical composition. Such carriers can influence the physical state, solubility, stability, rate of in vivo release, and rate of in vivo clearance of the active ingredient comprising the pharmaceutical composition.

The choice of carriers will further depend on whether the pharmaceutical composition is to be administered in solid, liquid or aerosol form, and whether it needs to be sterile for such routes of administration as injection. The compositions may be formulated into a composition in a free base, neutral or salt form. In embodiments where the composition is in a liquid form, a carrier can be a solvent or dispersion medium comprising but not limited to, water, ethanol, a polyol (e.g. glycerol, propylene glycol, liquid polyethylene glycol), lipids (e.g. triglycerides, vegetable oils, liposomes) and combinations thereof. Sterile injectable solutions are prepared by incorporating the active compounds in the required amount in the appropriate solvent with various of the other ingredients enumerated above, followed by filter sterilization.

The active ingredient of the pharmaceutical composition may be modified by covalent attachment of polymers such as polyethylene glycol, complexation with metal ions, or incorporation of the compound into or onto particulate preparations of polymeric compounds such as polylactic acid, polyglycolic acid, hydrogels, or onto liposomes, microemulsions, micelles, unilamellar or multi lamellar vesicles, erythrocyte ghosts, or spheroplasts.

The pharmaceutical composition of the invention may be provided in the form of controlled or sustained release composition. Controlled or sustained release compositions include formulation in lipophilic depots (e.g. fatty acids, waxes, oils). Also comprehended by the invention are particulate compositions coated with polymers (e.g. poloxamers or poloxamines). Other embodiments of the compositions of the invention incorporate particulate forms, protective coatings, protease inhibitors or permeation enhancers for various routes of administration.

Portions of the active ingredient may be labeled by association with a detectable marker substance (e.g. ¹²⁵I radiolabel or biotin) to provide reagents useful in detection and quantification of the active ingredient or its receptor, or its derivatives in solid tissue and fluid samples such as blood, cerebral spinal fluid or urine.

The pharmaceutical composition of the present invention can be formulated for administration by a route selected from: intravenously, intradermally, intraarterially, intraperitoneally, intralesionally, intracranially, intraarticularly, intraprostaticaly, intrapleurally, intratracheally, intranasally, intravitreally, intravaginally, intrauterinely, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

The preparation of pharmaceutical compositions comprising peptides is well known in the art, as disclosed for example in U.S. Pat. Nos. 5,736,519, 5,733,877, 5,418,219, 5,354,900, 5,298,246, 5,164,372, 4,900,549 and 4,457,917. Means for processing the pharmaceutical compositions of the present invention include, without limitations, conventional mixing, dissolving, granulating, grinding, pulverizing, dragee-making, levigating, emulsifying, encapsulating, entrapping or lyophilizing processes.

### Expression Vectors

The invention also provides an expression vector comprising an isolated polynucleotide sequence encoding a polypeptide domain of Necl4, or a fragment, analog or derivative thereof. The Necl4 can be human Necl4, and a polypeptide domain of human Necl4 includes an amino acid sequence selected from the group consisting of: SEQ ID NO:2, SEQ ID NO:3 SEQ ID NO:4 and SEQ ID NO:5. Necl4 domains of other species, which may be considered derivatives of human Necl4 domains, include SEQ ID NOS:6-13, 98 and 99. An isolated polynucleotide sequence encoding a human Necl4 domain or a derivative thereof may be selected from the group consisting of: SEQ ID NO:50, SEQ ID NO:51, SEQ ID NO:52; SEQ ID NO:53; SEQ ID NO:103 and SEQ ID NO:104. Examples of expression vectors directing expression of a polypeptide comprising a domain of Necl4 include: SEQ ID NO:84 (pCX-Necl4, for expression of an Fc-mouse Necl4 extracellular domain fusion protein); SEQ ID NO:89 (pGX-Necl4ct, for expression of a GST-Necl4 cytoplasmic domain fusion protein); SEQ ID NO:91 (pMX-GFP-Necl4ct, for expression of a GFP-Necl4 cytoplasmic domain fusion protein), and SEQ ID NO:95 (pMX-SSmyc-Ncl4dCT, for expression of a mouse/rat chimeric mutant Necl4 protein lacking the cytoplasmic domain) disclosed herein. The Necl4 cytoplasmic domain encoded and expressed by pGX-Necl4ct and by pMX-GFP-Necl4ct corresponds to SEQ ID NO:4 i.e the cytoplasmic domain of human Necl4.

The term “expression vector” refers to a vector containing a nucleic acid sequence coding for at least part of a gene product capable of being transcribed into RNA molecules which are then translated into a protein, polypeptide, or peptide. The vector may be a prokaryotic expression vector, a eukaryotic expression vector, a mammalian expression vector, a yeast expression vector, a baculovirus expression vector or an insect expression vector. Examples of such vectors include PKK233-2, pEUK-C1, pREP4, pBlueBacHisA, pYES2, PSE280 or pEBVHis.

In certain embodiments, the expression vector further comprises at least one regulatory element operatively linked to the polynucleotide. The term “regulatory element” as used herein refers to nucleic acid sequences necessary for the transcription and possibly translation of an operably linked coding sequence in a particular host organism. In addition to regulatory elements that govern transcription and translation, expression vectors may contain nucleic acid sequences that serve other functions as well. Examples of regulatory elements include a promoter, an enhancer, a selectable gene, a signal peptide, a recombinase gene, a transcription factor gene and a reporter gene.

The term “operatively linked” means that a regulatory element is in a correct functional location and/or orientation in relation to a nucleic acid sequence to control transcriptional initiation and/or expression of that sequence, or to provide another function as is provided by the particular regulatory element.

A “promoter” is a region of a nucleic acid sequence at which initiation and rate of transcription are controlled. It may contain genetic elements at which regulatory proteins and molecules may bind such as RNA polymerase and other transcription factors. A promoter may be one naturally associated with a gene or sequence, as may be obtained by isolating the 5′ non-coding sequences located upstream of the coding segment and/or exon. Such a promoter can be referred to as “endogenous.” Alternatively, certain advantages will be gained by positioning the coding nucleic acid segment under the control of a recombinant or heterologous promoter (examples include the bacterial promoters SP6, T3, and T7), which refers to a promoter that is not normally associated with a nucleic acid sequence in its natural environment. Promoters include promoters of other genes, promoters isolated from any prokaryotic, viral, or eukaryotic cell, and promoters which are not “naturally occurring” i.e. containing different promoter elements of different transcriptional regulatory regions, and/or mutations that alter expression. The promoter employed may be constitutive, tissue-specific, inducible, and/or useful under the appropriate conditions to direct high level expression of the introduced polynucleotide sequence, such as is advantageous in the large-scale production of recombinant proteins and/or peptides. A promoter may or may not be used in conjunction with an “enhancer”.

An “enhancer” is a cis-acting regulatory sequence involved in the transcriptional activation of a nucleic acid sequence. An enhancer may be one naturally associated with a nucleic acid sequence, located either downstream or upstream of that sequence. A recombinant or heterologous enhancer refers to an enhancer not normally associated with a nucleic acid sequence in its natural environment. Enhancers include enhancers of other genes, enhancers isolated from any prokaryotic, viral, or eukaryotic cell, and enhancers not “naturally occurring” i.e. containing different enhancer elements of different transcriptional regulatory regions, and/or mutations that alter expression

A “selectable gene” is a gene encoding a product which enables a cell or organism comprising the selectable gene (for example on an expression vector), to grow in a selective medium comprising a chemical which is normally toxic or lethal or non-metabolizable to the cell or organism lacking the product of the selectable gene. Examples of selectable genes include antibiotic resistance genes, the herpes simplex virus thymidine kinase gene, the hypoxanthine-guanine phosphoribosyltransferase gene, the dhfr gene which confers resistance to methotrexate, and the gpt gene which confers resistance to mycophenolic acid.

A “signal peptide”, also known as a leader peptide or a signal sequence, is a sequence which functions in directing migration of a nascent protein to the cell membrane and/or its extrusion through the membrane.

A “recombinase gene” is a gene encoding a recombinase enzyme which functions in catalyzing recombination between two DNA sequences. In genetic engineering, site-specific recombination systems have been developed to enable site specific ligation and/or excision of a particular DNA sequence within a target DNA sequence. Examples of recombinases encoded by recombinase genes include Cre recombinase, Flp recombination and R recombinase. Recombinases can be used for example, to remove a reporter or marker gene from a genetic construct, once the marker is no longer needed. Recombinase gene expression may be made inducible through the combined use of an induction system enabling conditional expression of the recombinase.

A “transcription factor gene” is a gene encoding a DNA-binding protein or a functional domain thereof. A transcription factor functional domain includes a DNA-binding domain and a transcriptional activation or suppression domain. A transcription factor gene can be included within an expression vector in order to enhance transcription.

A “reporter gene” is a gene encoding a molecule which by its chemical nature, provides an identifiable signal allowing detection of the polynucleotide. Detection can be either qualitative or quantitative. The present invention contemplates using any commonly used reporter genes as well as reporter molecules including radionuclides, enzymes, biotins, psoralens, fluorophores, chelated heavy metals, and luciferin. Commonly used enzymes include horseradish peroxidase, alkaline phosphatase, glucose oxidase and α-galactosidase. The substrates to be used with the specific enzymes are generally chosen because a detectably colored product is formed by the enzyme acting upon the substrate. For example, p-nitrophenyl phosphate is suitable for use with alkaline phosphatase conjugates; for horseradish peroxidase, 1.2-phenylenediamine, 5-aminosalicylic acid or toluidine are commonly used.

### Host Cells

The present invention provides a host cell expressing an exogenous domain of Necl4 or a fragment thereof. According to one embodiment, the host cell is selected from the group consisting of: a eukaryotic cell, a somatic cell, a germ cell, a neuronal cell, a pluripotent stem cell and a nerve progenitor cell. According to yet another embodiment, the neural cell is selected from the group consisting of: Schwann cell, myelinating Schwann cell, Schwann cell microvilli, glial cell and dorsal root ganglion neuron.

According to another embodiment, the host cell comprises an exogenous polynucleotide sequence encoding an amino acid sequence selected from the group consisting of: SEQ ID NO:2; SEQ ID NO:3, SEQ ID NO:4 and SEQ ID NO:5, which correspond to human Necl 4 domains. According to another embodiment, the host cell comprises an exogenous polynucleotide sequence encoding an amino acid sequence selected from the group consisting of: SEQ ID NO: 6; SEQ ID NO: 7, SEQ ID NO: 8; SEQ ID NO: 9; SEQ ID NO: 10; SEQ ID NO: 11; SEQ ID NO: 12; SEQ ID NO: 13; SEQ ID NO: 98 and SEQ ID NO: 99. The polynucleotide sequence may be selected from the group consisting of: SEQ ID NO:50, SEQ ID NO:51, SEQ ID NO:52; SEQ ID NO:53; SEQ ID NO:103 and SEQ ID NO:104.

The host cell may be a human cell which has been stably transformed by a recombinant nucleic acid molecule encoding a domain of Necl4 or an active fragment thereof. The nucleic acid may be operatively linked to a regulatory element as defined herein including but not limited to a promoter, an enhancer, a selectable gene, a signal peptide, a recombinase gene, a transcription factor gene and a reporter gene. In one embodiment, the expression vector has a nucleotide sequence selected from the group consisting of: SEQ ID NO:84; SEQ ID NO:89; SEQ ID NO:91 and SEQ ID NO:95.

### Antibodies

The present invention provides an anti-Necl4 antibody that recognizes, binds and is specific for a Necl4 domain or a fragment thereof. Antibodies of the invention can be produced using any method well known and routinely practiced in the art.

As used herein an isolated Necl4 domain includes an amino acid sequence selected from the group consisting of: SEQ ID NOS:2-13. Antibody fragments, including Fab, Fab′, F(ab′)₂, and Fv, are also provided by the invention. The terms “specific for” and “directed to” synonymously indicate that the variable regions of the antibodies of the invention recognize and bind Necl4 and/or domains thereof exclusively, but may also interact with other proteins (for example, other antibodies in ELISA techniques) through interactions with sequences outside the variable region of the antibodies, and in particular, in the constant region of the molecule.

Non-human antibodies may be humanized by any methods known in the art. In one method, the non-human complementarity determining regions (CDRs) are inserted into a human antibody or consensus antibody framework sequence. Further changes can then be introduced into the antibody framework to modulate affinity or immunogenicity.

For example, U.S. Pat. No. 5,585,089 discloses a humanized immunoglobulin and methods of preparing same, wherein the humanized immunoglobulin comprises complementarity determining regions (CDRs) from a non-human donor immunoglobulin and heavy and light chain variable region frameworks from human acceptor immunoglobulin heavy and light chains. U.S. Pat. No. 5,225,539 also discloses an altered antibody or antigen-binding fragment thereof and methods of preparing same, wherein a variable domain of the antibody or antigen-binding fragment has the framework regions of a first immunoglobulin heavy or light chain variable domain and the complementarity determining regions of a second immunoglobulin heavy or light chain variable domain, wherein said second immunoglobulin heavy or light chain variable domain is different from said first immunoglobulin heavy or light chain variable domain in antigen binding specificity, antigen binding affinity, species, class or subclass.

Antibodies of the invention have utility in therapy of conditions responsive to modulation of Necl4 or a domain thereof; diagnosis of conditions characterized by aberrant levels of Necl4 or a particular domain thereof; quantitation of isolated Necl4 or a domain thereof; and purification of Necl4 or a domain thereof. Antibodies are useful for detecting and/or quantitating Necl4 expression in cells, tissues, organs and lysates and extracts thereof, as well as fluids, including serum, plasma, cerebrospinal fluid, urine, sputum, peritoneal fluid, pleural fluid, or pulmonary lavage. Kits comprising an antibody of the invention for any of the purposes described herein are also comprehended. In general, a kit of the invention also includes a control antigen for which the antibody is immunospecific.

Specific binding proteins can be identified or developed using isolated or recombinant Necl4 products, Necl4 variants, or cells expressing such products. Binding proteins are useful for purifying Necl4 products and detection or quantification of Necl4 products in fluid and tissue samples using known immunological procedures. Binding proteins are also manifestly useful in modulating (i.e. blocking, inhibiting or stimulating) biological activities of Necl4, especially those activities involved in myelination of neuronal cells.

Anti-idiotype antibodies specifically immunoreactive with an antibody of the invention are also contemplated.

Small interfering RNA (siRNA)

The present invention provides a siRNA molecule that is capable of down regulating expression of Necl4 via RNA interference (RNAi). RNAi is a process by which a particular RNA sequence is targeted and destroyed upon activation of cellular enzymes, as a result of complementarity between a siRNA and the particular RNA sequence. Both nuclear and cytoplasmic RNA can be targeted. When messenger RNA transcripts of a particular gene are targeted and destroyed, the expression of the corresponding gene product is reduced or eliminated.

RNAi is a multi-step process mediated by double stranded RNA (dsRNA). dsRNA activates post-transcriptional gene expression surveillance mechanisms that appear to function to defend cells from virus infection and transposon activity. (Fire et al., 1998; Grishok et al., 2000; Ketting et al., 1999; Lin et al., 1999; Montgomery et al., 1998; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene.

According to certain embodiments, the siRNA molecule of the invention useful for down regulating Necl4 expression is a double stranded RNA (dsRNA) molecule comprising two separate RNA strands in which one strand has at least one region complementary to a region on the other strand, or a single stranded molecule comprising a hairpin loop wherein at least one region of the hairpin loop is complementary to an opposing region of the hairpin loop. The complementarity region of such a hairpin loop may be referred to as the stem.

According to a certain embodiment, the siRNA of the invention is a dsRNA wherein: (a) each strand of the siRNA molecule is independently about 15 to about 30 nucleotides in length; and (b) one strand of the siRNA molecule comprises a nucleotide sequence having a degree of complementarity to a Necl4 RNA sufficient to induce Necl4 RNA interference.

According to certain embodiments, the siRNA molecule comprises an oligonucleotide selected from SEQ ID NO:55, SEQ ID NO:56 and SEQ ID NO:57.

As used herein, complementarity between an siRNA and a target nucleic acid sequence means that at least a region (i.e. the complementarity region) of the siRNA can bind to the target nucleic acid sequence in a sequence-specific manner, according to the dictates of nucleic acid base pairing. The complementary regions allow sufficient hybridization of the siRNA to the target RNA and thus permit cleavage.

Lengths of siRNA may be referred to in terms of bases, which refers to the length of a single strand, or in terms of basepairs, which refers to the length of the complementarity region. According to certain embodiments, the siRNA molecule is about 100 bases or less in length (or has 100 basepairs or fewer in the complementarity region). In specific embodiments, the strand or strands of the siRNA are less than about 70 bases in length. With respect to those embodiments, the siRNA strand or strands may be from about 55 to about 70, from about 45 to about 60, from about 30 to about 45, from about 15 to about 30 and from about 5 to about 30 bases or basepairs in length. A siRNA that has a complementarity region equal to or less than about 30 basepairs (such as a single stranded hairpin RNA in which the stem or complementary portion is less than or equal to about 30 basepairs) or one in which the strands are about 30 bases or fewer in length is specifically contemplated. Thus, a hairpin siRNA (one strand) may be 70 or fewer bases in length with a complementary region of about 30 basepairs or fewer.

The degree of complementarity between the siRNA and the target RNA, or between the complementarity region of the siRNA and the target RNA, can be 100% i.e. fully complementary, or less than 100%. Thus, “complementary” sequences include sequences that are at least 50% complementary, and may be at least 50%, 60%, 70%, 80%, or 90% complementary. It is contemplated that in some instances siRNA generated from sequence based on one organism may be used in a different organism to achieve RNAi of the cognate target gene. For example, siRNA generated from a dsRNA that corresponds to a human Necl4 gene may be used in a mouse cell to mediate RNAi of Necl4 expression, if there is the requisite complementarity in mediating RNAi.

Ultimately, the requisite degree of complementarity of the siRNA according to the invention is dictated by its functional capability in mediating RNAi and thus down regulating Necl4 expression in a particular cell, organ or tissue system.

The complementary strands or regions can comprise mismatches. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

In certain embodiments, the siRNA is a dsRNA comprising a nucleotide overhang, for example, a 2 nucleotide overhang. The nucleotide overhang can be at the the 3′ or 5′ or both ends of the dsRNA. The nucleotide overhang can include any combination of a thymine, uracil, adenine, guanine, or cytosine, or derivatives or analogs thereof.

The siRNAs of the invention can be made by many methods. In particular aspects, the siRNAs are chemically synthesized or are an analog of a naturally occurring siRNA.

In some embodiments of the invention, the siRNA comprises at least one modification, such as a 2′-sugar modification, a nucleic acid base modification, or a phosphate backbone modification. Such chemical modifications can enhance the stability of siRNA molecules against degradation by serum ribonucleases. International PCT Publication Nos. WO 91/03162; WO 92/07065; WO 93/15187; WO 97/26270 and WO 98/13526 and U.S. Pat. Nos. 5,334,711; 5,627,053; and 5,716,824 describe various chemical modifications that can be made to sugar, nucleic acid base and/or phosphate moieties. An siRNA having any modified residue, derivative or analog is encompassed by the invention to the extent that it does not eliminate or substantially reduce the ability of the siRNA to mediate RNAi with regard to Necl4 mRNA and down regulating of Necl4 expression.

While chemical modification of oligonucleotide internucleotide linkages with phosphorothioate, phosphorothioate, and/or 5′-methylphosphonate linkages improves stability, too many of these modifications can cause some toxicity. Therefore when designing siRNA molecules, the amount of internucleotide linkages should be minimized. The reduction in the concentration of these linkages should lower toxicity resulting in increased efficacy and higher specificity of these molecules.

The present invention further provides a method of down regulating Necl4 expression in a cell, comprising contacting the cell with the siRNA molecules of the invention, under conditions suitable for down regulating of Necl4 expression. According to one embodiment, the suitable conditions include but are not limited to the presence of a divalent cation, for example Mg²⁺. In another embodiment, the siRNA comprises at least one modification selected from the group consisting of: a 2′-sugar modification, a nucleic acid base modification, and a phosphate backbone modification. In another embodiment, the siRNA comprises an oligonucleotide selected from SEQ ID NO:55, SEQ ID NO:56 and SEQ ID NO:57.

By “down regulate” or “inhibit” it is meant that the expression of a Necl4 gene, or the level of Necl4 RNA or equivalent RNA encoding a full length Necl4 or a domain or fragment thereof is eliminated or reduced to a level less than that observed in the absence of the siRNA molecule of the invention. In one embodiment, the degree of down regulation or inhibition achieved with the siRNA molecule is greater than that achieved in the presence of an enzymatically inactive or attenuated control molecule. In other words, the Necl4 gene expression is higher in the presence of the inactive or attenuated control molecule. An inactive or attenuated control molecule can include for example, a scrambled sequence, a sequence that is able to bind to the same site on the target RNA, but is unable to induce cleavage of the target RNA, and a sequence having an insufficient degree of complementarity. The effect of the siRNA on expression of Necl4 or of a domain thereof, in a cell treated with siRNA can be assessed using immunodetection methods known in the art, for example immunofluorescence using antibodies directed against the full length Necl4 protein or a Necl4 domain of interest. Cells having down regulation of Necl4 mediated by a siRNA according to the invention, will exhibit reduced or eliminated specific immunofluorescence, compared to control cells treated with an inactive dsRNA. Alternately, expression of Necl4 or of a domain thereof, in a cell treated with siRNA according to the invention, can be assessed using a biological assay, for example, a receptor binding assay. For assessment of Necl4 expression, an example of an appropriate receptor is Necl1. Cells having down regulation of Necl4 mediated by a siRNA according to the invention, will exhibit reduced or eliminated specific receptor binding, compared to control cells treated with an inactive dsRNA.

Any cell that undergoes RNAi can be employed in the method of down regulating Necl4 expression. The cell may be a eukaryotic cell, a mammalian cell such as a primate, rodent, rabbit, or human cell, a prokaryotic cell, or a plant cell. In some embodiments, the cell is alive, while in others the cell or cells is in an organism or tissue. Alternatively, the cell may be dead. The dead cell may also be fixed. In some cases, the cell is attached to a solid, non-reactive support such as a plate or petri dish. Such cells may be used for array analysis. It is contemplated that cells may be grown on an array and dsRNA administered to the cells.

The siRNA and the method of down regulating Necl4 expression according to the invention have utility in investigating Necl4 function and its mechanism of action. For example, cells having down regulated Necl4 expression can be used for identification and characterization of Necl4 receptors, and for assessment of the role of Necl4 in myelination and neural cell development. Accordingly there is also disclosed a method of examining the function of a Necl4 gene in a cell or organism comprising (a) introducing siRNA of from about 5 to about 30 nucleotides that targets mRNA of the Necl4 gene for degradation into the cell or organism, thereby producing a test cell or test organism; (b) maintaining the test cell or test organism under conditions under which degradation of Necl4 mRNA occurs, thereby producing a test cell or test organism in which Necl4 mRNA is degraded; and (c) observing the myelination or neural cell receptor binding phenotype of the test cell or test organism produced in (b). The method can further comprise comparing the myelination or neural cell receptor binding phenotype observed to that of an appropriate control cell or control organism, thereby providing information about the function of Necl4 with respect to myelination or neural cell development.

The siRNA and the method of down regulating Necl4 expression according to the invention have utility in treating a disease or condition associated with aberrant myelination. Aberrant myelination includes demyelination and excessive myelination. According to the disclosures herein, individual domains of Necl4 can have opposing effects on the extent of myelination of neuronal cells. Accordingly, there is also disclosed a method of treating a disease or condition associated with aberrant myelination in an subject comprising introducing to the subject siRNA of from about 5 to about 30 nucleotides that targets mRNA of a Necl4 gene or a fragment thereof for degradation.

The siRNA and the method of down regulating Necl4 expression according to the invention have further utility in assessing whether an agent acts on a Necl4 gene product, including a Necl4 domain. Accordingly there is also disclosed a method of assessing whether an agent acts on a Necl4 gene product comprising: (a) introducing siRNA of from about 5 to about 30 nucleotides which targets the Necl4 mRNA for degradation into a cell or organism; (b) maintaining the cell or organism of (a) under conditions in which degradation of the Necl4 mRNA occurs; (c) introducing the agent into the cell or organism of (b); and (d) determining whether the agent has an effect on the cell or organism, for example with respect to myelination, wherein if the agent has no effect on the cell or organism then the agent acts on the Necl4 gene product or on a biological pathway that involves the Necl4 gene product.

The invention further provides a method of inhibiting Necl4 activity in a cell, the method comprising down regulating expression of Necl1 via RNA interference with an siRNA molecule specific for Necl1. According to one embodiment, the siRNA molecule specific for Necl1 comprises the oligonucleotide of SEQ ID NO:57. It is disclosed herein (see Example 6) that down-regulating the expression of Necl4 expression in a cell via siRNA specific for Necl4 results in decreased binding of Necl1. In a reciprocal experiment, down-regulating the expression of Necl1 expression in a cell via siRNA specific for Necl1 results in decreased binding of Necl4. Accordingly, when inhibition of the biological activity of Necl4 is required, it may be advantageous to also target Necl1 i.e. the binding partner of Necl4.

The terms “siRNA”, “dsRNA” and “DNA” are used interchangeably herein to describe nucleic acid molecules with enzymatic activity, and include similar technologies such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme and DNA enzyme. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it has nucleotide sequences within or surrounding that substrate binding site which impart a nucleic acid cleaving and/or ligation activity to the molecule (e.g. U.S. Pat. No. 4,987,071).

Methods for the delivery of siRNA molecules are described in Akhtar et al., 1992, Trends Cell Bio. 2, 139; and Delivery Strategies for Antisense Oligonucleotide Therapeutics, ed. Akhtar, 1995 both incorporated herein by reference. WO 94/02595 further describes the general methods for delivery of siRNA molecules. siRNA molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules and bioadhesive microspheres. Alternatively, the nucleic acid/vehicle combination is locally delivered by direct injection or by use of an infusion pump. More detailed descriptions of nucleic acid delivery and administration are provided in WO93/23569; WO99/05094, and WO99/04819 all of which have been incorporated by reference herein.

The present invention further provides methods of administering the siRNA molecules of the invention to a cell. The cell can be a mammalian cell (e.g. human cell), wherein the cell can be in culture or in a mammal. The siRNA molecules of the invention are added directly, or can be complexed with cationic lipids, packaged within liposomes, or otherwise delivered to target cells or tissues.

The siRNA can be locally administered to relevant tissues ex vivo, or in vivo through injection or infusion pump, with or without their incorporation in biopolymers.

The siRNA molecules of the invention may be expressed from transcription units inserted into DNA or RNA vectors. The recombinant vectors are preferably DNA plasmids or viral vectors. The expressing viral vectors can be constructed based on, but not limited to, adeno-associated virus, retrovirus, adenovirus, or alphavirus. Preferably, the recombinant vectors are capable of delivering and expressing the siRNA molecules in target cells. Alternatively, viral vectors can be used that provide for transient expression of the siRNA molecules. Such vectors can be repeatedly administered as necessary. Once expressed, the siRNA molecules bind to the target RNA and down-regulate its function and/or expression. Delivery of the expressing vectors can be systemic, such as by intravenous or intramuscular administration, by administration to target cells explanted from the patient or subject followed by reintroduction into the patient or subject, or by any other means that would allow for introduction into the desired target cell.

### Methods of Treatment

The present invention provides several methods of treating a neurological disorder in a subject in need thereof. The neurological disorder can comprise nerve demyelination or excessive myelination associated with either or both of the peripheral nervous system and the central nervous system. The neurological disorder can be associated with a neural injury or disease. Neural injury can be associated with physical trauma to either or both of the peripheral nervous system and the central nervous system, or to defective development, either in utero or following birth. Clinical conditions and diseases comprising nerve demyelination include immune defects, inherited disorders, metabolic disorders, mitochondrial defects, toxin or radiation exposure and infectious diseases. Examples of diseases comprising nerve demyelination include but are not limited to acute inflammatory demyelinating polyradiculoneuropathy, chronic inflammatory demyelinating polyradiculoneuropathy, chronic idiopathic axonal polyneuropathy, adrenoleukodystrophy, diabetic neuropathy, Guillain-Barre disease (acute demyelinating polyneuropathy), multiple sclerosis, HIV inflammatory demyelinating disease and post infection encephalomyelitis. Examples of diseases comprising excessive myelination include but are not limited to meningioangiomatosis, Lhermitte-Duclas disease and Vogt's syndrome.

According to one embodiment, the method of treatment comprises administering to the subject a pharmaceutical composition comprising a therapeutically effective amount of a polypeptide comprising an isolated domain of Necl4 or an active fragment, analog or derivative thereof. Administering is typically carried out by oral administration, intravenous injection, intramuscular injection or intrathecal injection.

According to particular embodiments, the polypeptide comprises an amino acid sequence selected from the group consisting of: SEQ ID NO:2, SEQ ID NO:3, SEQ ID NO:4 and SEQ ID NO:5.

For pharmaceutical use, the polypeptides of the present invention are formulated for parenteral, particularly intravenous or subcutaneous, delivery according to conventional methods. Intravenous administration will be by bolus injection or infusion over a typical period of one to several hours. As detailed above, pharmaceutical formulations will include a Necl4 domain or an active fragment thereof in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art (see for example, Remington's Pharmaceutical Sciences, 18th Ed. Mack Printing Company, 1990).

Therapeutic doses will generally be in the range of 0.1 to 100 μg/kg of patient weight per day, preferably 0.5-20 μg/kg per day, with the exact dose determined by the clinician according to accepted standards determination of dose is within the level of ordinary skill in the art. The proteins may be administered for acute treatment, over one week or less, often over a period of one to three days or may be used in chronic treatment, over several months or years.

According to another embodiment, the method of treatment comprises transplanting the host cells of the invention in a subject in need thereof. The transplanted host cells may be autologous cells genetically modified to express a Necl4 domain or an active fragment thereof. Preferably, the host cells are transplanted at or near at least one predetermined locus. In particular embodiments, the host cells comprise a polynucleotide sequence selected from the group consisting of SEQ ID NO:50; SEQ ID NO:51; SEQ ID NO:52; SEQ ID NO:53 and SEQ ID NO:103.

It is possible to carry out the methods of the invention by autologous transplantation. Cells are removed from the body and the vector is introduced thereto as a naked DNA plasmid to form transformed cells which are then re-implanted into the body. Naked DNA vector for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter.

According to yet another embodiment, the method of treatment comprises introducing the expression vectors of the invention to the subject by means of gene therapy. In one embodiment, in the vector is a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes virus 1 (HSV1) vector, an attenuated adenovirus vector, such as the vector described by Stratford-Perricaudet et al., J. Clin. Invest. 90:626-630 (1992), and a defective adeno-associated virus vector.

In another embodiment, the vector is a retroviral vector, e.g., as described in Anderson et al., U.S. Pat. No. 5,399,346; U.S. Pat. Nos. 4,650,764; 4,980,289; U.S. Pat. No. 5,124,263; and International Patent Publication No. WO 95/07358.

The vector can be introduced by lipofection in vivo using liposomes. Synthetic cationic lipids can be used to prepare liposomes for in vivo transfection of a gene encoding a marker. The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

The invention further provides a pharmaceutical composition comprising host cells expressing an exogenous domain of human Necl4 or a fragment, analog or derivative thereof for treating neurological damage. The invention further provides a pharmaceutical composition comprising a polypeptide comprising an isolated domain of human Necl4 or a fragment, analog or derivative thereof for treating neurological damage. The invention further provides a pharmaceutical composition comprising an expression vector comprising an isolated polynucleotide sequence encoding a polypeptide domain of human Necl4, or a fragment, analog or derivative thereof for treating neurological damage.

Diagnostic Assays

The antibodies of the invention can be used for the purpose of detecting, diagnosing or monitoring a neurological defect. Such antibodies can be polyclonal or monoclonal, or prepared by molecular biology techniques.

Antibodies can be labeled with a variety of detectable labels including, but not limited to, radioisotopes, ultrasound contrast agents and paramagnetic metals. Targetable diagnostic and/or therapeutically active agents, particularly ultrasound contrast agents are disclosed in U.S. Pat. No. 6,680,047.

In general labeling moieties should be compatible with the detection method that is applied. Thus, for positron emission tomography (PET) radionuclides are typically used. The radionuclide of choice may be selected from F-18, Mn-51, Mn-52m, Fe-52, Co-55, Cu-62, Cu-64, Ga-68, As-72, Br-75, Br-76, Rb-82m, Sr-83, Y-86, Zr-89, Tc-94m, In-110, I-120, and I-124.

In magnetic resonance imaging techniques the agent should comprises a suitable metal, optionally the metal is associated with a chelating agent such as DTPA and DOTA and the like. Metals that can be used as the diagnostic moiety for detection with MRI techniques are selected from the group consisting of gadolinium, manganese, iron, chromium, copper, cobalt, nickel, dysprosium, rhenium, europium, terbium, holmium and neodymium.

For gamma-ray detection the diagnostic moiety bound to the antibody of the invention may be any one or more of the following: Cr-51, Co-57, Co-58, Fe-59, Cu-67, Ga-67, Se-75, Ru-97, Tc-99m, In-111, In-114m, I-123, I-125, I-131, Yb-169, Hg-197, and T1-201.

Thus, for instance, a diagnostic assay in accordance with the invention for measuring levels of Necl4 compared to normal control bodily fluids, cells, or tissue samples may be used to diagnose the neurological damage.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

In order that this invention may be better understood, the following examples are set forth. These examples are for the purpose of illustration only and are not to be construed as limiting the scope of the invention in any manner.

### EXAMPLES

### Experimental Procedures

### REX-SST Library Construction

Poly-A+-RNA was isolated from dbcAMP-stimulated rat Schwann cells or 3-day-old rat sciatic nerves using FastTrack 2.0 kit (Invitrogen) according to the manufacturer's instructions. The original pMX-SST-vector (a generous gift from Y. Kitamura, University of Tokyo, Japan) was modified slightly by introducing EcoRI and XhoI sites to the multiple cloning site to allow directional cloning of cDNAs. cDNA was synthesized with the cDNA synthesis kit (Stratagene) using custom-made random-primers containing an XhoI-site. The cDNAs were size-selected on ChromaSpin TE-400 columns (Clontech), ligated into the EcoRI-XhoI digested REX-SST-vector and electroporated into ElectroMax DH10B-cells (Invitrogen). The primary libraries (5×10⁵ cfu for the iSC-library and 1.7×10⁶ cfu for the 3drSN-library) were titrated and amplified by growing 2.5×10⁴ clones on 15-cm agar LB-Amp plates overnight at 37° C. Plasmid DNA was prepared and used for transfection of Phoenix-Eco packaging cells to prepare viral stocks.

Screening and Isolation of cDNA-Inserts

Screening of the REX-SST libraries was done as previously described (Kojima and Kitamura, 1999). Ba/F3-cells (2−6×10⁷) were infected with the iSC- and the 3drSN-retroviral libraries and grown in the presence of interleukin 3 (IL-3). After 24 hours, the infected Ba/F3-cells were washed three times in RPMI 1640 medium without IL-3, seeded in 96-well plates at a density of 3.3×10³ well⁻¹ and grown for 10 days in selection medium without IL-3. Surviving clones were transferred to new 96-well plates, and confluent wells were passaged three further times. Cells were then lysed in lysis-buffer (10 mM Tris-HCl pH 7.5, 200 mM NaCl, 1 mM EDTA, 1.7 μM SDS, 0.5 mg ml⁻¹ Proteinase K) at 56° C. in a humid chamber, followed by heat inactivation at 85° C. for 20 minutes. Lysed cells (3 μl) were used for PCR (5′-primer, GAAGGCTGCCGACCCCG; SEQ ID NO:70); 3′-primer, GGCGCGCAGCTGTAAACG; SEQ ID NO:71) to isolate the cDNA-inserts, and the resulting products were separated on agarose gels. When two or more PCR products were detected, additional PCR was performed on the respective bands using the same primers. Pre-screening for highly abundant genes was done by spotting the PCR-products onto Hybond+nylon membranes and hybridizing them to a mix of P32-labeled probes derived from clones representing four genes: osteonectin (bp 11-356; D28875), collagen 1α1 (bp 1-405; Z78279), collagen 18α1 (bp 15-586; AK031798) and tyrosinase-related protein 1 (bp 330-867; XM_{—}238398). Hybridization-negative PCR-products were purified and sequenced using the original 5′ primer of the PCR.

### Tissue Culture Methods

Culture of primary rat Schwann cells, purified DRG neurons and dissociated myelinating DRG-cultures were done as previously described (Eshed et al., 2005). Viral infection was carried out for two hours with 8 μg/ml polybrene on the first, second and third days after plating with undiluted viral stock; myelination was induced after 10 days in culture and proceeded for additional 10-12 days before analysis. Cell-lines (COS-7, 293T and Phoenix-Eco) were grown in DMEM/10% FCS. Fc-fusion proteins were produced in HEK 293T cells as described previously (Gollan et al., 2003). COS-7 cells were transfected with LipofectAMINET reagent (GIBCO BRL).

### Constructs and Antibodies

The plasmids used are detailed in Table 1. Retroviral expression-constructs were made in pMX (Kojima and Kitamura, 1999). Viral stocks were prepared using the helper-virus-free Phoenix-Eco packaging cells (kindly provided by G. Nolan, Stanford, Calif.). Plasmid pMX-SSmyc-Necl4FLdirects expression of a mutant Necl4 molecule (SEQ ID NO: 46), corresponding to the full length rat Necl4 in which the native signal sequence is replaced by the human immunoglobulin kappa chain signal sequence (corresponding to SEQ ID NO:48), and which further has a myc tag addition (SEQ ID NO:49) near the N-terminus. Plasmid pMX-SSmyc-Necl4dCT directs expression of a mutant Necl4 molecule (SEQ ID NO: 47) corresponding to a rat Necl4 molecule lacking the cytoplasmic domain, and in which the native signal sequence is replaced by the human immunoglobulin kappa chain signal sequence (corresponding to SEQ ID NO:48), and which further has a myc tag addition (SEQ ID NO:49) near the N-terminus.

Polyclonal antibodies to isolated domains of Necl1 and Necl4 were generated by immunizing rabbits with GST-fusion proteins containing the cytoplasmic (ct) domains of rat Necl1 and rat Necl4, respectively. Affinity-purified antibodies were prepared by first absorbing each serum on a column containing the non-immunizing GST-fusion (e.g. GST-Necl4ct for anti-Necl1ct serum and GST-Necl1ct for anti-Necl4ct serum) and then purifying the unbound antibodies on a column containing the GST-fusion protein used for immunization. Antibodies to gliomedin (Eshed et al., 2005), Caspr (Peles et al., 1997; Poliak et al., 1999) were previously described. The following antibodies were obtained from commercial sources: rat anti-MBP (Chemicon), mouse anti-neurofilaments (Sigma), mouse anti-MAG (clone 513; Roche), rabbit and mouse anti-S100 (Sigma), mouse anti-Na⁺ channels (Sigma), and mouse anti-bIII Tubulin (Covance).

TABLE 1
Plasmid Constructs.

| | | | | SEQ ID | | | |
|---|---|---|---|---|---|---|---|
| | | | | NO of | | | |
| Plasmid | Gene | Species | Accession | insert | Vector | 5′ seq | 3′ seq |
| pGEM-Necl1-ISH | partial | rat | XM_341157 | 72 | pGEM-T | GGGCCAAT | CCCTCCAATG |
| | Necl1 | | | | Easy | CTTTCCCA | ATGGCGTGG |
| | | | | | | GG | |
| pGEM-Necl2-ISH | partial | rat | NM_001012201 | 73 | pGEM-T | GCCGCGGC | CACCGGTAC |
| | Necl2 | | | | Easy | CCAGCCGG | CTCCACTGCC |
| | | | | | | CCACAGGT | CCAATGGCG |
| | | | | | | GATGGGCA | |
| | | | | | | GAATC | |
| pGEM-Necl3-ISH | partial | mouse | NM_178721 | 74 | pGEM-T | GCACGCTA | AGCAGGATC |
| | Necl3 | | | | Easy | GCGTTCGC | CGGGCCATTC |
| | | | | | | TCAACCAG | TGGCCAGCC |
| | | | | | | CATCTC | |
| pGEM-Necl4-ISH | partial | rat | XM_344870 | 75 | pGEM-T | GAGGGTGG | GGTCAAGAT |
| | Necl4 | | | | Easy | GGTGGCTG | GGAGGGAAG |
| | | | | | | AG | GG |
| pC3-Necl1 | Necl1 | rat | XM_341157 | 76 | pCDNA3 | CGCCGCTA | CGCACTCGA |
| | | | | | | GCAAGCTT | GGTGTGGGT |
| | | | | | | GGGAGGTG | GCCCCTAGAT |
| | | | | | | GCCAGGAA | G |
| | | | | | | GC | |
| pC3.1-Necl3- | Necl3 | mouse | NM_178721 | 77 | pCDNA3.1- | GCACGCTA | CTGAGGATC |
| myc/his | | | | | myc/his | GCGTTTCGC | CAATGAAAT |
| | | | | | | TCAACCAG | ACTCTTTTTT |
| | | | | | | CATCTC | CTCTTCG |
| pC3.1-Necl4 | Necl4 | mouse | NM_153112 | 78 | pCDNA3.1- | GTATCTGC | CAATCTCGA |
| | | | | | myc/his | AGATAACG | GGCTGGCTCC |
| | | | | | | GCACC | TTGCAGCTGG |
| pSX-Necl1 | Necl1 | rat | XM_341157 | 80 | pSX-Fc | GGGCGGCC | GGCGGGATC |
| | | | | | | CAGCCGGC | CGTACTGGA |
| | | | | | | CAATCTTT | GGATGAGGG |
| | | | | | | CCCAGGAC | CAC |
| pSX-Necl2 | Necl2 | mouse | NM_207675 | 81 | pSX-Fc | CGGCGGCC | GTCCGGATCC |
| | | | | | | CAGCCGGC | CCAATGGTCC |
| | | | | | | CCCCACAG | CCTCTTC |
| | | | | | | GTGATGGA | |
| | | | | | | CAG | |
| pCX-Necl3 | Necl3 | mouse | NM_178721 | 82 | pCX-Fc | GCACGCTA | AGCAGGATC |
| | | | | | | GCGTTCGC | CGGGCCATTC |
| | | | | | | TCAACCAG | TGGCCAGCC |
| | | | | | | CATCTC | |
| pCX-Necl4 | Necl4 | mouse | NM_153112 | 83 | pCX-Fc | TGGCGCTA | AATGAGATC |
| | | | | | | GCAAGCTT | TGGAACCGA |
| | | | | | | GCCACCAT | TGTCTGAGCC |
| | | | | | | GGGCCGGG | TC |
| | | | | | | CCC | |
| pGX-Necl1ct | Necl1 | rat | XM_341157 | 85 | pGEX- | ATTGAATT | GTGGCTCGA |
| | | | | | 6P1 | CGGACACT | GCTAGATGA |
| | | | | | | ATTTGATC | AATATTCCTT |
| | | | | | | CGGCAC | CTTGTC |
| pGX-Necl2ct | Nec12 | rat | NM_001012201 | 86 | pGEX- | TTTGGAAT | GAACCTCGA |
| | | | | | 6P1 | TCATTCTG | GGCTGATCTA |
| | | | | | | GGCCGCTA | GATGAAGTA |
| | | | | | | TTTTGC | CTC |
| pGX-Necl3ct | Necl3 | mouse | NM_178721 | 87 | pGEX- | CTCCGAAT | GAACCTCGA |
| | | | | | 6P1 | TCCTGCTT | GCCTGAATTT |
| | | | | | | GGCCGATA | TAAATGAAA |
| | | | | | | TC | TACTC |
| pGX-Necl4ct | Necl4 | rat | XM_344870 | 88 | pGEX- | ATGGAATT | GGGACTCGA |
| | | | | | 6P1 | CTGCTCTG | GTCAAATGA |
| | | | | | | TCCGACAG | AGAACTCTTC |
| | | | | | | AAGG | TTTCCG |
| pMX-GFP- | Necl4 | rat | XM_344870 | 90 | pMX- | ATGGAATT | GGGACTCGA |
| Necl4ct | | | | | GFP-C | CTGCTCTG | GTCAAATGA |
| | | | | | | TCCGACAG | AGAACTCTTC |
| pMX-SSmyc- | Necl4 | mouse/ | NM_153112/ | 92 | pMX | 5′ SSmyc | 3′ SSmyc |
| Necl4FL | | rat | XM_344870 | | | GCTTAGAT | TATCGCATGC |
| | | | | | | CTGCTAGC | TCAGATCCTC |
| | | | | | | GTCGCCAC | TTCTGAGATG |
| | | | | | | CATGGAGA | AGTTTTTGTT |
| | | | | | | CAGACAC | CGGGATCCA |
| | | | | | | 5′ Necl4FL | AGCTTCGTAC |
| | | | | | | CAGGGCAT | GG |
| | | | | | | GCAGACCG | 3′ Necl4FL |
| | | | | | | AGAATGTG | CTGCGTCTGC |
| | | | | | | ACTGTG | TTGCTGTGC |
| pMX-SSmyc- | Necl4 | mouse/ | NM_153112/ | 94 | pMX | 5′ SSmyc: | 3′ SSmyc: |
| Necl4dCT | | rat | XM_344870 | | | GCTTAGAT | TATCGCATGC |
| | | | | | | CTGCTAGC | TCAGATCCTC |
| | | | | | | GTCGCCAC | TTCTGAGATG |
| | | | | | | CATGGAGA | AGCTTTTTGTT |
| | | | | | | CAGACAC | CGGGATCCA |
| | | | | | | 5′ Necl4dCT: | AGCTTCGTAC |
| | | | | | | CAGGGCAT | GG |
| | | | | | | GCAGACCG | 3′ Necl4dCT: |
| | | | | | | AGAATGTG | CTCGCTCGAG |
| | | | | | | ACTGTG | GGCTACCAG |
| | | | | | | | ACCATGCCC |
| | | | | | | | ACTAGC |

### RNA Expression Analysis

Semi-quantitative RT-PCR was performed using the primers listed in Table 2. Total RNA was extracted with TriReagent (Sigma) and cDNA was prepared with the ImProm II Reverse Transcription System (Promega) using PolyT-primers; the amounts of the resulting cDNA were normalized using actin-specific primers. Northern blot analysis of Necl4 was performed as previously described (Poliak et al., 2002) using the same probe that was used for in situ hybridizations. In situ hybridization (ISH) was performed using cRNA probes SEQ ID NOS:72-75 for Necl1-4, respectively (from pGEM-Necl-ISH plasmids listed in Table 1). Hybridization was performed at very stringent hybridization-conditions (71.5° C.) as previously described (Spiegel et al., 2002).

TABLE 2
RT PCR Primers

| Gene | 5′ seq | 3′ seq |
|---|---|---|
| Rat Necl1 | CTCGGAATCCCACAGAAACC | GCTGAGAGGTGGATCTGTC |
| | (SEQ ID NO: 58) | (SEQ ID NO: 59) |
| Rat Necl2 | CGATATCCAGAAAGACACGG | CTGCACTTCTAGATACCGCTG |
| | (SEQ ID NO: 60) | (SEQ ID NO: 61) |
| Rat Necl3 | CTTTTCCACAAGAAGGACAGG | CTGCGCTGCTTTGACCAATG |
| | (SEQ ID NO: 62) | (SEQ ID NO: 63) |
| Rat Necl4 | CGTGGAAATGGGAATTCTG | GGTCAAGATGGAGGGAAGGG |
| | (SEQ ID NO: 64) | (SEQ ID NO: 65) |
| Rat Gliomedin | AGAGAGTCTGCTAACAGGAG | GGTATGTGGTATTGATGTGC |
| | (SEQ ID NO: 66) | (SEQ ID NO: 67) |
| Rat Actin | GAGCACCCTGTGCTGCTCACCGAGG | GTGGTGGTGAAGCTGTAGCCACGCT |
| | (SEQ ID NO: 68) | (SEQ ID NO: 69) |

### Immunoprecipitation and Immunoblot Analysis

Brain membrane preparation, immunoprecipitation (IP), and immunoblot analysis were done as described previously (Poliak et al., 2003). For immunopreciptation of Necl1 and Necl4 from rat brain membranes, membranes were solubilized in SB-buffer and lysate of one rat brain was used for twelve IP's; for immunoprecipitation from Schwann cells, one confluent 10 cm plate was solubilized in SB, centrifugated twice for 10 min at 14′000 rpm at 4° C. and the resulting supernatant used for one IP. For immunoblot-analysis of Necl4, two cleaned rat sciatic nerves were crushed in 400 μl of 25 mM Tris, pH 8.0, and 1 mM EDTA 2% SDS, boiled for 15 min, and spun for 15 min at 15° C.

### Immunofluorescence

Teased sciatic nerves were fixed for 10 min in Zamboni's fixative and postfixed/permeabilized in cold methanol for 5 min at −20° C. Slides were blocked for 1 hour with PBS containing 5% normal goat serum and 0.1% Triton X-100 (blocking solution). The samples were incubated overnight at 4° C. with different primary antibodies diluted in blocking solution; washed with PBS and then incubated for 45 min at room-temperature with secondary anti-mouse-Cy3 (Jackson Laboratories), anti-rabbit-488 (Molecular Probes), and anti-rat-Cy5 (Jackson laboratory). Slides were mounted with elvanol and analyzed using a Nikon Axioplan microscope or a BioRad confocal microscope. Myelinated cultures were fixed for 15 min in 4% PFA, permeabilized for 10 min in ice-cold methanol at −20° C., blocked for 1 hour at room-temperature and then processed as above.

### Fc-Fusion Binding, Clustering and Perturbation Experiments

Binding experiments were done by incubating the cells with medium containing different the Fc-fusions proteins pre-incubated with anti human Fc-Cy3 as described previously (Eshed et al., 2005). For clustering experiments, purified DRG-neurons or isolated Schwann cells were incubated with medium containing the respective Fc-fusion protein as described above, washed once and grown for additional 24 (DRG) or 4 hours (Schwann) before fixing. Fc-perturbation experiments were preformed by adding 50 mg/ml purified proteins to the medium of dissociated DRG cultures 2 days before the induction of myelination. Fresh medium containing the Fc-fusion proteins was replaced every second day and the cultures were fixed and stained after 11-12 days of myelination.

siRNA and Transfection of Schwann Cells

RNA-oligonucleotides were purchased from Dharmacon and handled according to the manufacturers instruction; stock-solutions of all siRNAs were at a concentration of 20 mM. The following target sequences were used for rat Necl4: CTATAGTCGTCATTCAGAATT (SEQ ID NO:55) and GCCCAAGGCCGATGAAATCTT (SEQ ID NO:56). For rat Necl1, the following target sequence was used: CCAGCAGACTCTATACTTT (SEQ ID NO:57). For transfection, one confluent 10 cm Primaria-plate of rat Schwann cells was divided onto two 24-well plates containing PLL-coated 12 mm-coverslips with SCPM-medium without antibiotic. The next day, immediately prior to transfection, the medium was changed to DMEM alone at a volume of 200 ml per well. For transfection of 4 wells, the following was prepared: one tube containing 12 ml of Optimem I (Gibco) and 3 ml Oligofectamine (Gibco) and another tube with 180 ml Optimem 1 and 15 ml of the respective RNA-oligonucleotide. After 5 minutes at room temperature, the two tubes were mixed and kept at room temperature in the dark for another 20 minutes; after that, 50 ml of the transfection-mix were added to each well (final concentration of the RNA-oligonucleotide: 100 nM). After four to five hours, 125 ml of SCPM (without antibiotic) ×3 were added to each well. The next day, the medium was changed back to SCPM containing antibiotic and the cells were grown until they were assayed.

### Adhesion-Assay of Rat Schwann Cells to Purified Fc-Fusion Proteins

The adhesion-assay was done as previously described (Adamsky et al., 2001). Substrates were prepared by placing 2 ml containing of 50 mg/ml of proteins for 1 hour on a non-tissue culture plastic dish followed by three washes and blocking with 2% of heat-inactivated BSA for 1 hour. In competition experiments, the second Fc-fusion protein was applied before the blocking step. 2×10⁴ Schwann cells were allowed to adhere for 2 hours at room-temperature; non-adhering cells were then washed away with PBS and the remaining cells were fixed with 4% PFA and stained with Crystal Blue; low-magnification images were acquired with a CCD camera, and the cells were counted using the ImageJ software (downloadable from http://rsb.info.nih.gov/ij/). Experiments were repeated 3 times with duplicates for each Fc-fusion in each experiment. The amount of cells bound in each set of duplicates and repetition was normalized with the amount of cells bound to Necl1-Fc representing 100%; ANOVA-analysis was made using the Instat3-software (GraphPad).

### Quantification of Myelination Assays

Myelination was assayed by counting the amount of all MBP-positive segments on a coverslip. Stained coverslips were manually screened in a fluorescence microscope at low magnification using equal settings of the CCD camera for all coverslips in a given experiment. Pictures were taken of all MBP-positive segments on a coverslip and MBP-positive segments were counted with an in house-developed application for MatLab7.0 (MathWorks; the application can be received upon request). All myelination experiments were repeated two to three times with three to four samples per treatment. To asses the statistical significance of differences between the means of various treatments we performed Welch's test for a two-sample comparison of means with unequal variances between the averages with and without treatment (Ref: “Biometry”, Robert R. Sokal, F. James Rohlf, W. H. Freeman; 3rd edition); Welch's test was used since the variances of the different treatments could not be assumed to be equal. A conservative Bonferroni correction was used for multiple hypothesis testing.

### Electron Microscopy of Myelinating Cultures

Cultures grown on coverslips were washed 3 times in Kosnovsky-fixative (2% gluteraldehyde, 3% PFA, 3% sucrose, in 0.1M cacodylate-buffer), fixed in the same fixative for 2.5 hours at room temperature and additional 48 hours at 4° C., washed 4 times in 0.1M cacodylate-buffer and kept at 4° C. until further use. Ossification was made in 1% OsO₄, 0.5% K₂Cr₂O₇, 0.5% K₄[Fe(CN)₆]±3H₂O, 3% sucrose (in 0.1M cacodylate-buffer) for 2 hours at room temperature before being washed twice in 3% Sucrose (in 0.1M cacodylate-buffer) and three times in DDW. For enhancement, cultures were impregnated with 2% uranyl-acetate for 1 hour. Dehydrated samples were incubated with increasing concentrations of Epon (“hard”). Glass coverslips were removed by treatment for 2 hours in 30% fluoric acid, washed in DDW and dried overnight in an oven. The Epon-blocks of the cultures were then roughly cut into small pieces and re-embedded. Finally, 70-100 nm ultra-thin slices were cut from each block, mounted onto grids and analyzed in a CM-12 Philips electron-microscope equipped with a BioCam CCD-camera.

### Demyelination and Intraneural Injection of Fusion Proteins

All experiments involving animals were performed in accordance with the National Institutes of Health Guidelines for the human treatment of animals. Adult Sprague Dawley rats were anesthetized, and the sciatic nerve was exposed. Nerves were injected with 2-3 μl of 1% lysolecithin in sterile Locke's solution (pH 7.4) by using a glass micropipette. To mark the injection sites and observe the filling of each nerve, 0.05% Fast Green (Sigma, St. Louis, Mo.) was included in the lysolecithin solution. Each incision was then closed and rats were returned to cages for recovery. Five, and then again eight days, after the original lysolecithin injection, rats were anesthetized and each demyelinated site was injected with 3 ml of 3 mg ml⁻¹ human Fc or Necl4-Fc (3 rats for each fusion protein) diluted in PBS containing 0.05% Fast Green. Each incision was then closed and rats were returned to cages for recovery. Finally, eleven days after the initial injection of lysolecithin the rats were killed and the injected nerves were collected and fixed for 30 min using 4% paraformaldehyde. Nerves were cryoprotected overnight in 20% sucrose, cut in 16 μm thick sections, and immunostained as described in Schafer et al., 2004.

### Example 1

### Isolation of Necl Molecules Using a Signal Sequence Trap

In order to isolate cell surface molecules that mediate axon-glial interactions at the onset of myelination and during maintenance of the myelin sheath, a signal sequence trap technique was used to screen a Schwann cell library and a sciatic nerve cell library, as previously described (Spiegel et al. 2006). The rationale of the technique is that signal sequences, which direct cell surface proteins to the cell surface, can be detected in cDNA fragments, based on the ability to redirect a constitutively active mutant of the thrombopoeitin receptor MPL (MPLM) to the cell surface, thereby permitting IL-3-independent growth of Ba/F3 cells, which, otherwise, require IL-3 for survival (Kojima and Kitamura, 1999). The retroviral REX-SST contains a truncated MPLM variant that lacks most of the extracellular domain, including the signal sequence. Cloning a cDNA that contains a signal sequence in frame to the MPLM sequence will direct the expression of this fusion protein to the plasma membrane and result in IL 3-independent growth of Ba/F3-cells.

Two cDNA-libraries were constructed in the REX-SST vector. The first library was made using PolyA+RNA extracted from rat primary Schwann cells that were stimulated with the cAMP analog dibutyryl cAMP (dbcAMP) to induce their differentiation. For the second library, sciatic nerves of 3 day old rats were used as the source of RNA. Both of these sources are expected to be enriched in mRNAs that might be important for myelination. Treating Schwann cells with dbcAMP mimics axonal contact and induces the genetic myelination program (Morgan et al., 1991). Accordingly, stimulating primary Schwann cells with dbcAMP should generate a highly enriched source of mRNAs expressed in Schwann cells once they have contacted their axons. The 3 day old sciatic nerve was selected because it represents a stage when most Schwann cells have already contacted the axons and begun to myelinate (Friede and Samoraj ski, 1968), thereby allowing the identification of proteins that are expressed during the early, active period of myelination.

Sequence analysis of 863 isolated clones resulted in the identification of 158 cDNAs. Of these 158 cDNAs, 28 (18%) were found in both Schwann cells and sciatic nerve. Among these, several cell adhesion molecules were isolated, with immunoglobulin superfamily (IgSF) members constituting the largest subgroup. Two members of this subgroup have been described previously in the nervous system: Necl1 at non junctional contact sites between neuron and glia in the CNS and PNS (Kakunaga et al., 2005); and Necl2 (also termed SynCAM1), which induces synaptogenesis in the CNS (Biederer et al., 2002). The third member of this group, Necl4, was hitherto uncharacterized with respect to its function in the nervous system.

The rat Necl4 gene is disclosed under GenBank Accession No. XM_{—}344870 and under GenBank Accession No. NM_{—}001047107.

### Example 2

### Members of the Necl Family are Found in the Peripheral Nervous System

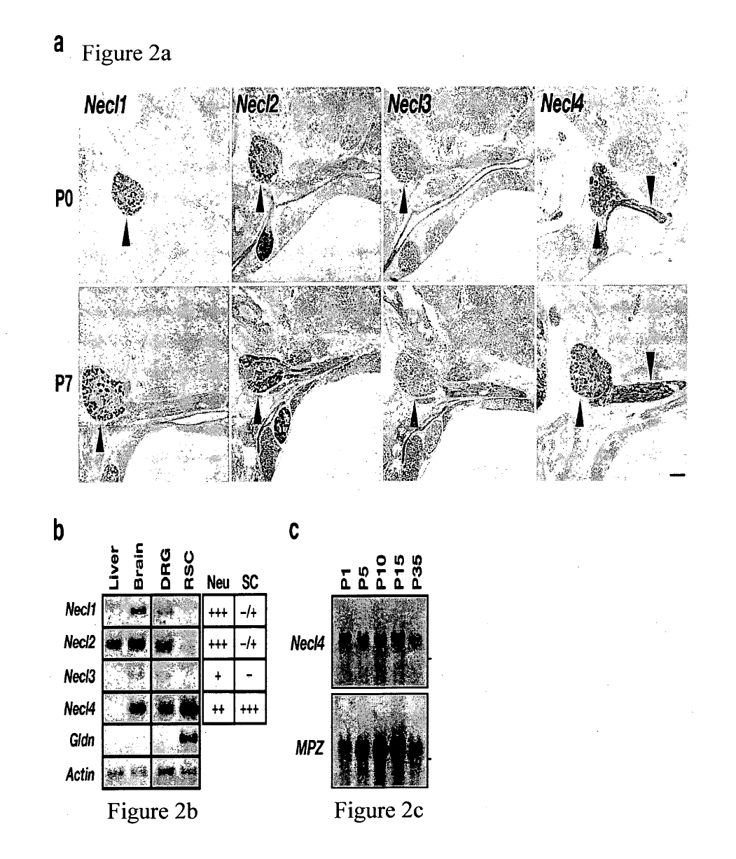

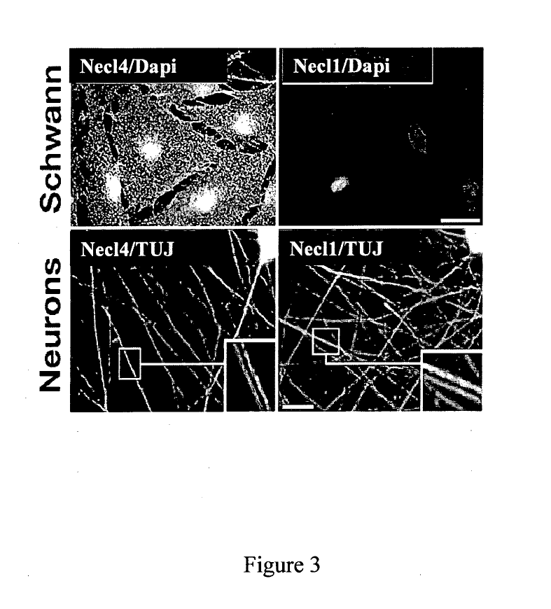

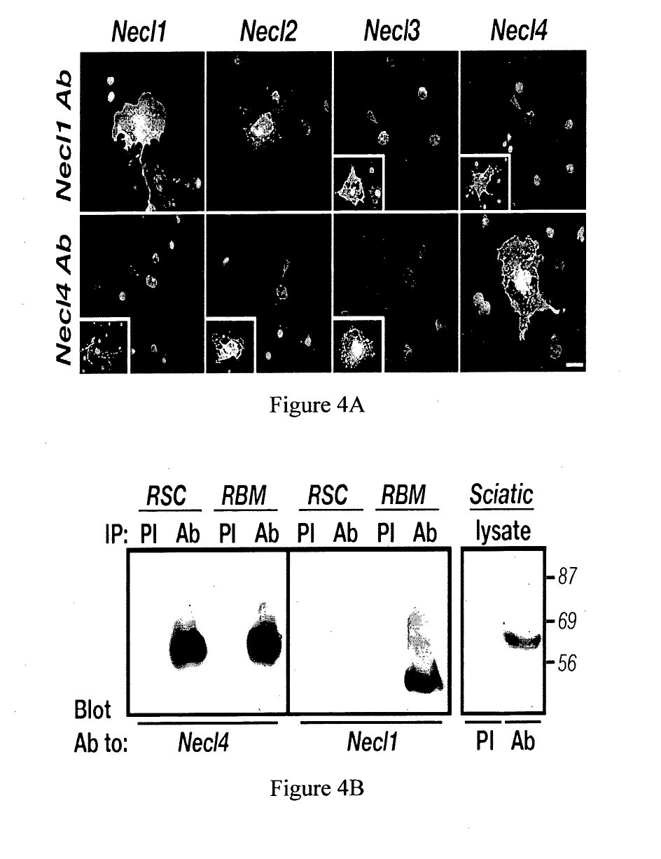

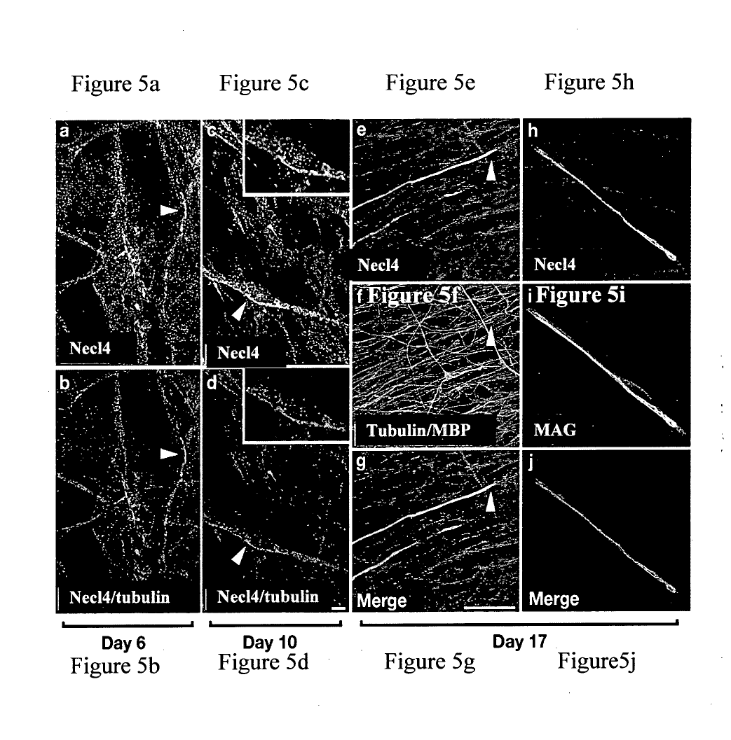

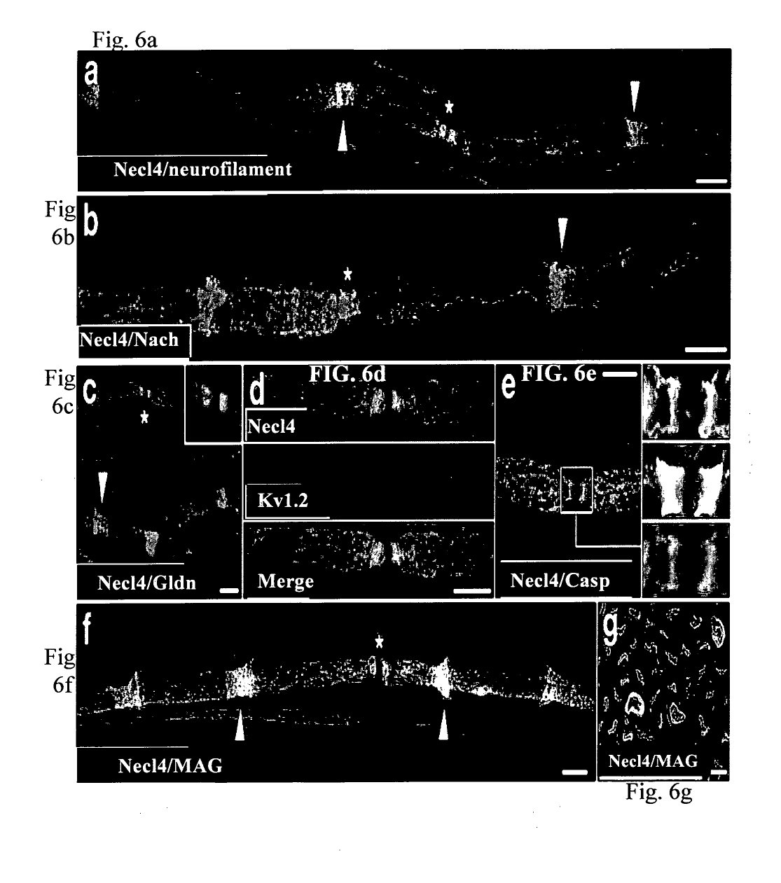

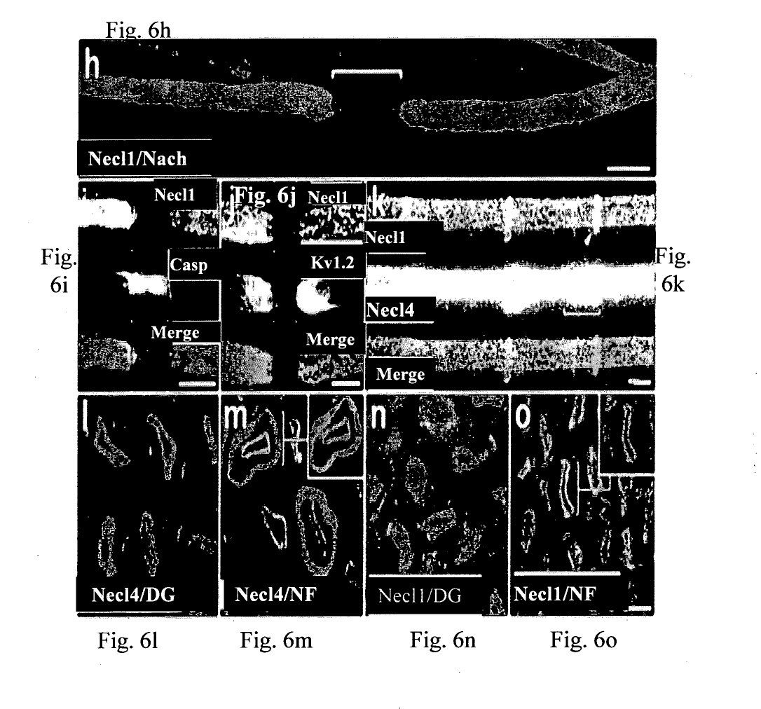

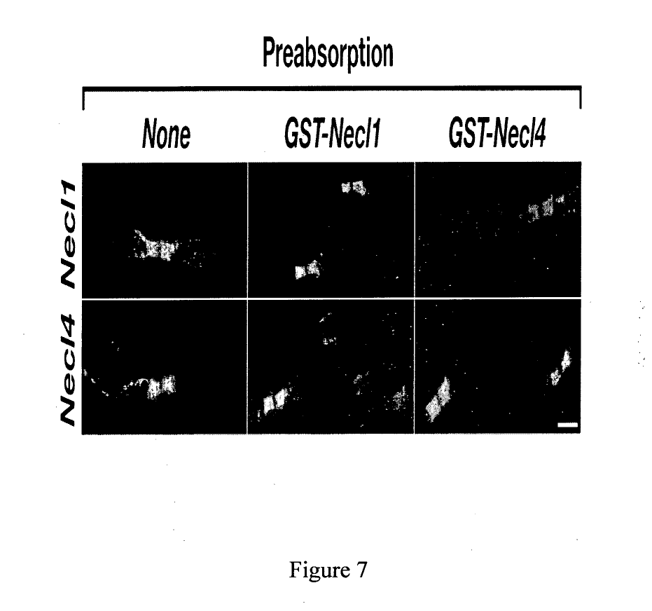

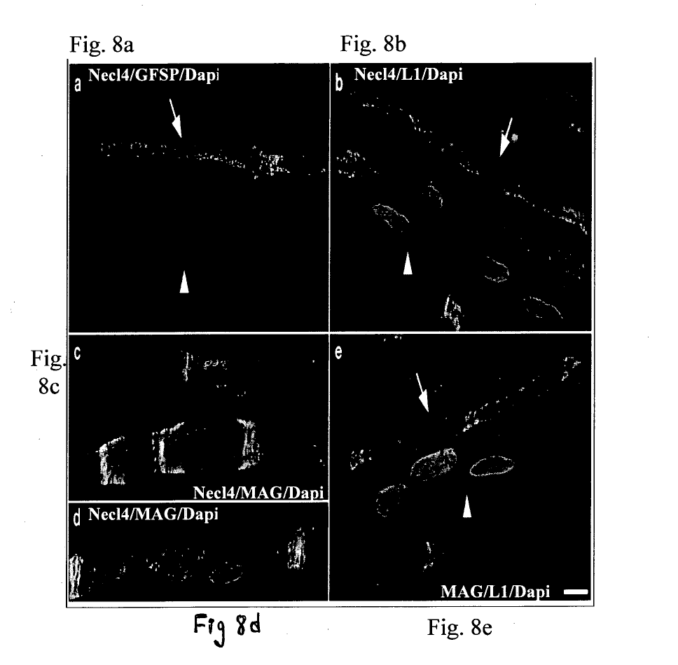

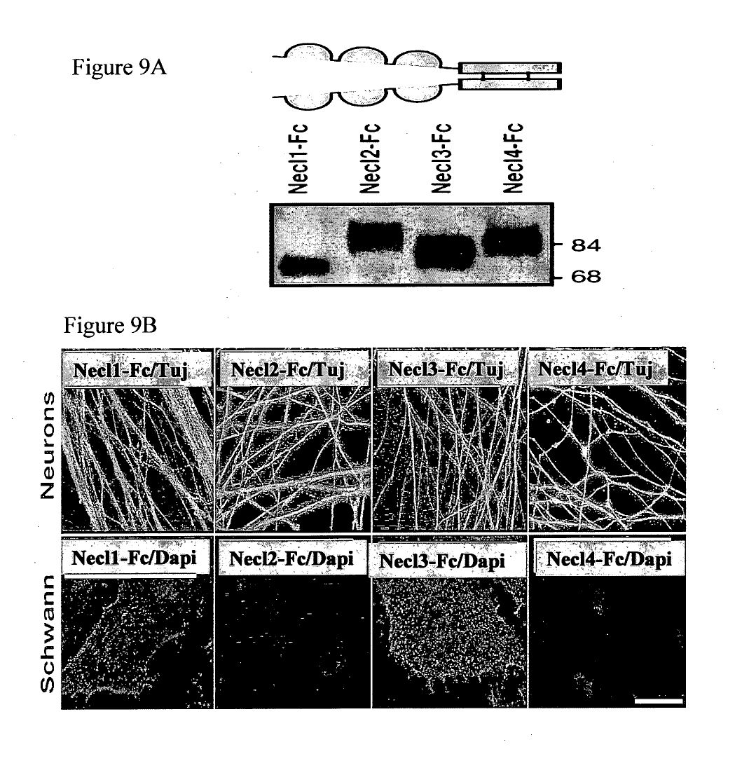

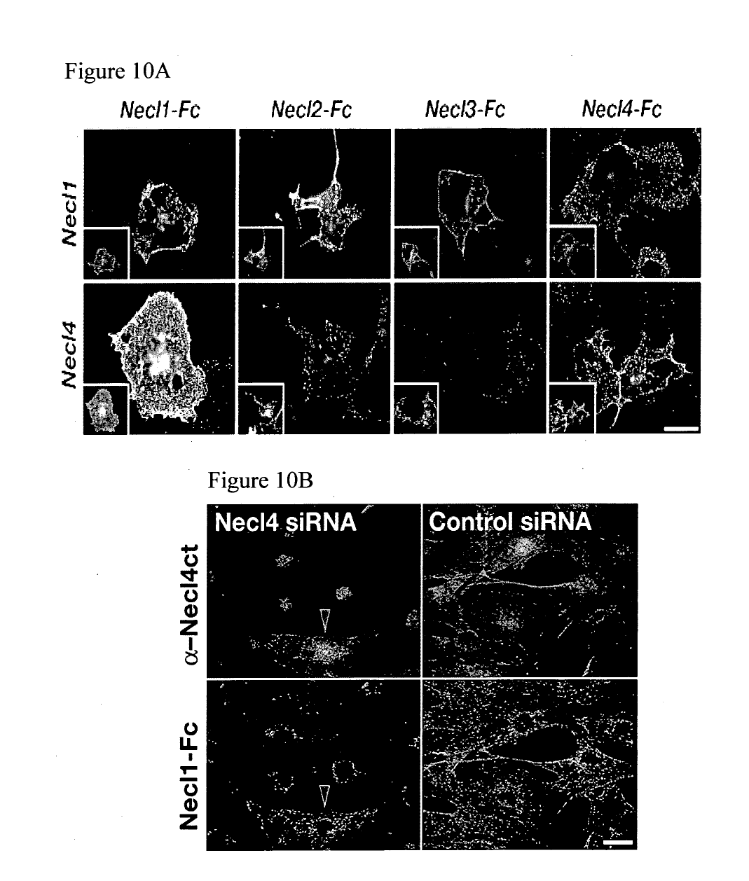

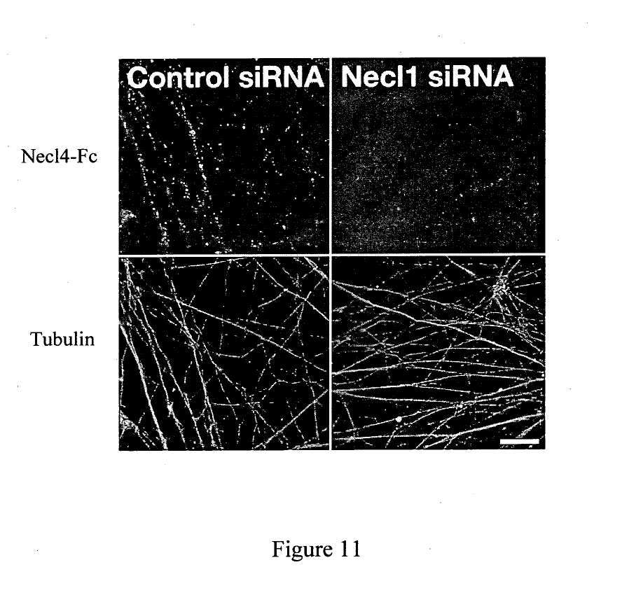

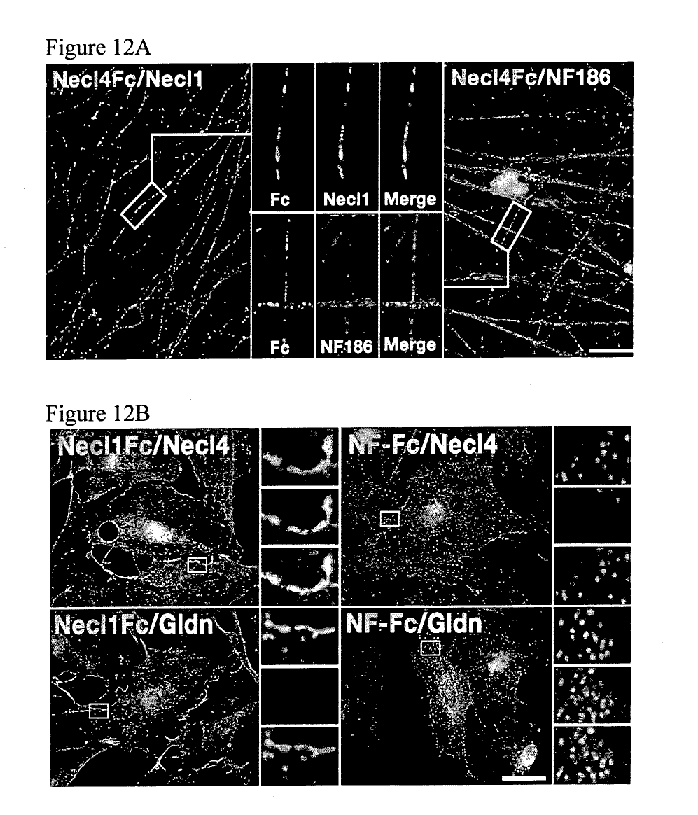

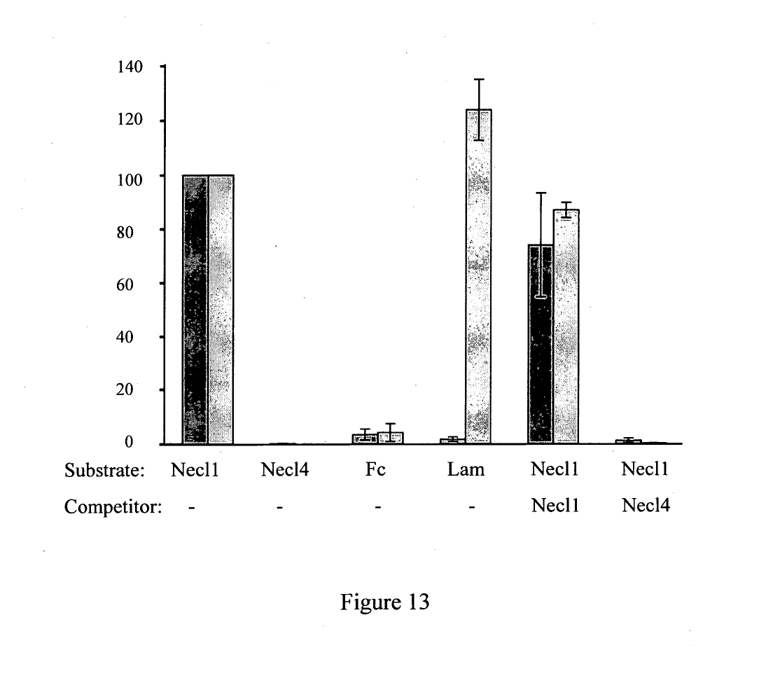

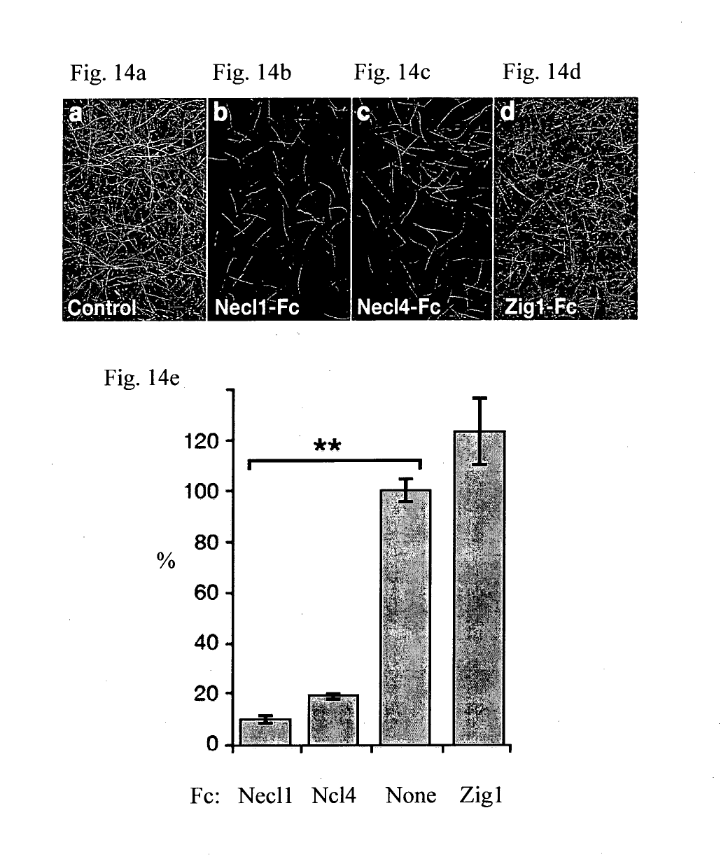

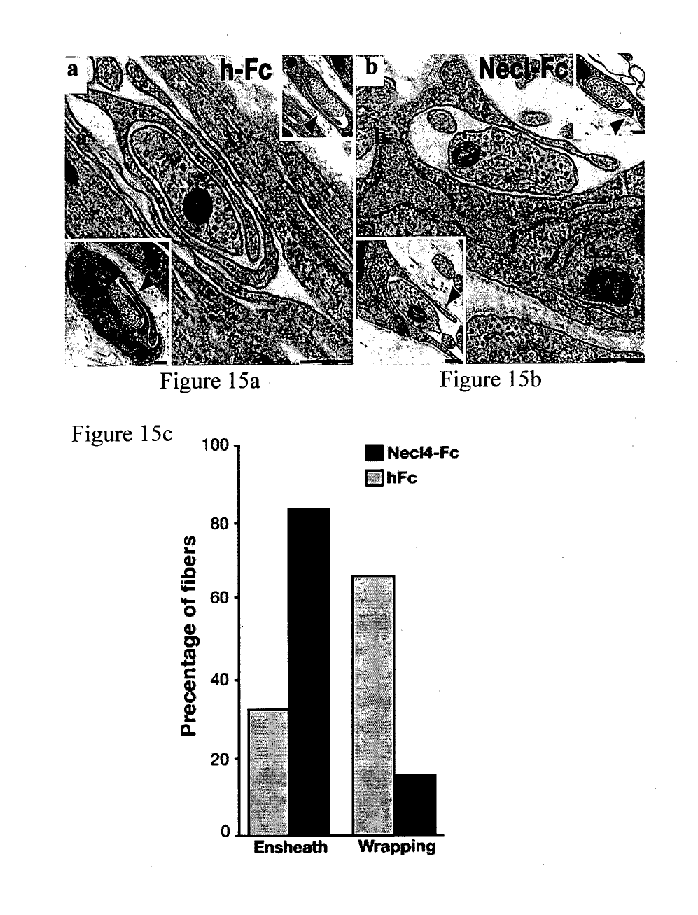

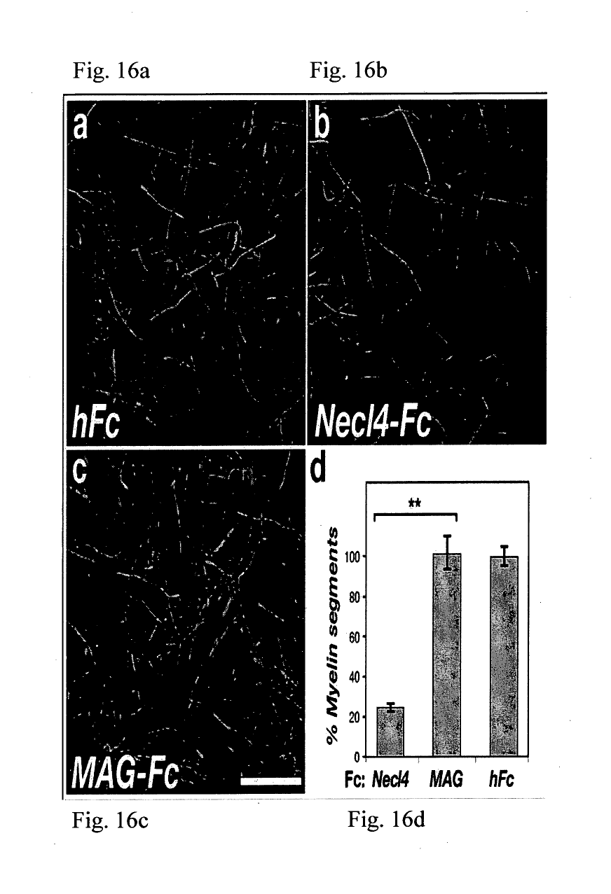

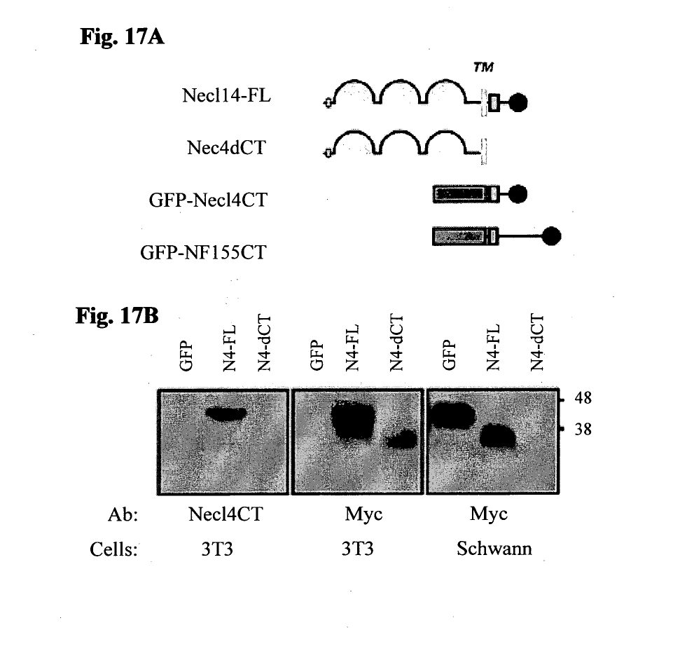

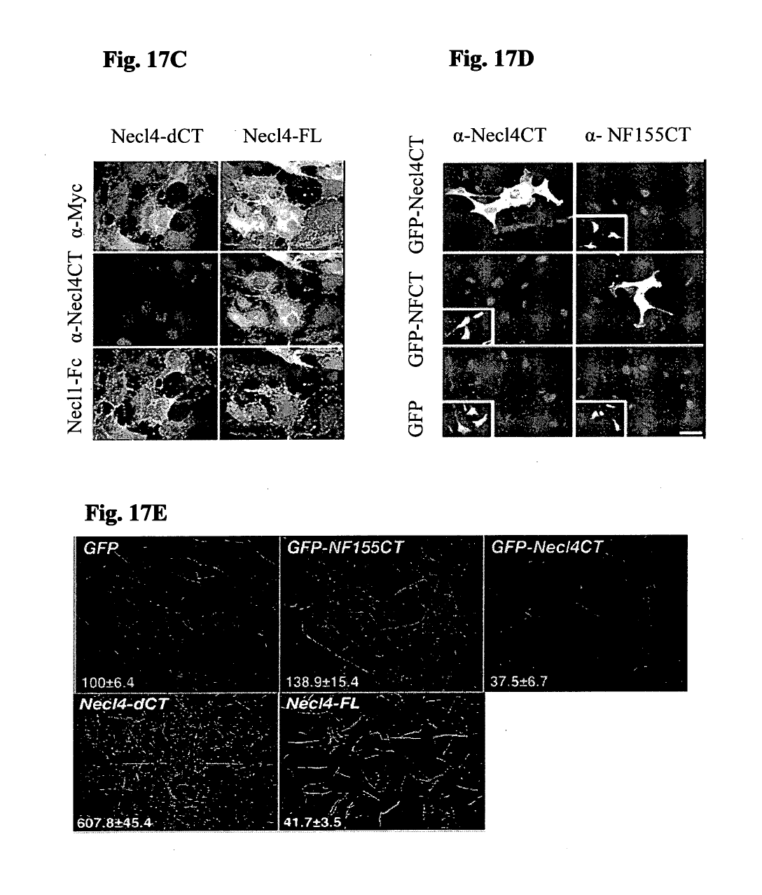

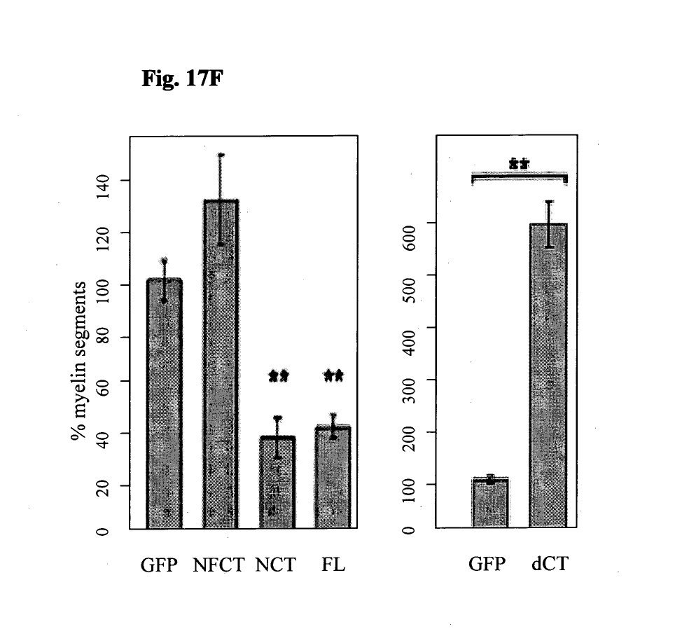

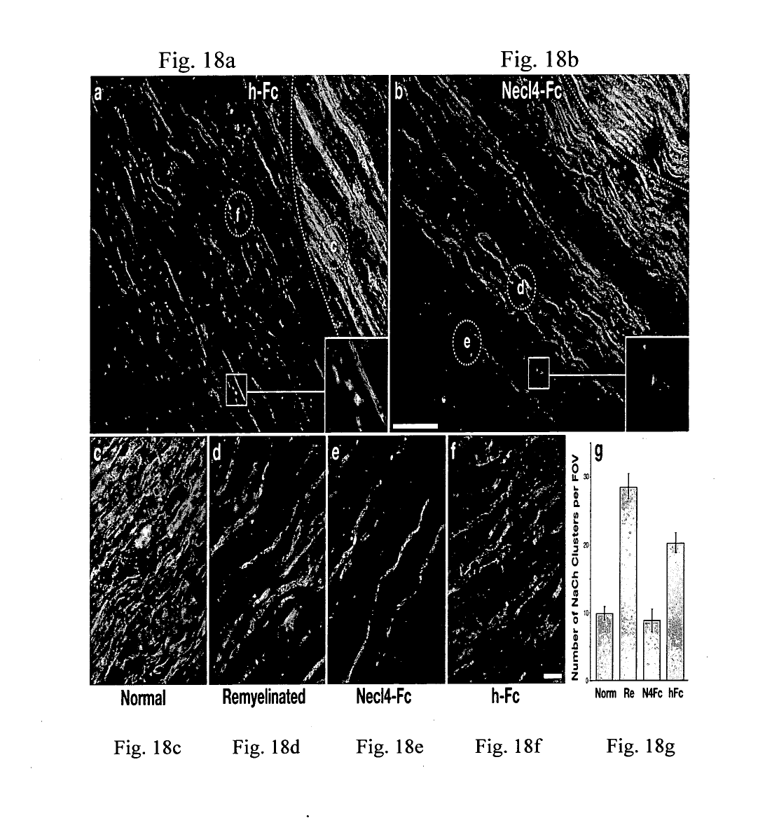

To examine the expression of Necls in the PNS, we performed in situ hybridization of newborn and 7-day old rats using specific probes for Necl1, Necl2, Necl3 and Necl4 (FIG. 2a). Necl1, Necl2 and Necl4 were clearly detected in dorsal root ganglia. In contrast to the other Necl2, Necl4 was also detected in Schwann cells located along the nerve. The expression of Necl4 dramatically increases in myelinating Schwann cells during the first postnatal week, which corresponds to the initial period of active myelination in the PNS.

RT-PCR analysis on mRNA isolated from cultured dorsal root ganglia (DRG) neurons and Schwann cells showed that while transcripts of all four Necls could be detected in DRG neurons, cultured Schwann cells express high levels of Necl4 and low levels of Necl2, but do not express Necl1 or Necl3 (FIG. 2b).

Northern blot analysis of sciatic nerve from different postnatal days demonstrated that Necl4 mRNA expression increases during the first two weeks after birth in a manner that is reminiscent of other myelin-related genes such as Myelin P) (MPZ; FIG. 2c).

An antiserum against Necl 4 that specifically recognizes Necl 4 but not Necl1-Necl3 was generated, as well as an antiserum that recognizes Necl1 and slightly Necl2 but not Necl3 or Necl4 (further described in Example 3). Cultured rat Schwann cells immunostained with these antibodies had strong Necl4-immunoreactivity in the cell membrane and processes, but were Necl1-negative (FIG. 3). Staining of isolated DRG neurons revealed high levels of Necl1 and weaker levels of Necl4 on the cell soma and along the axons.

As summarized in the table in FIG. 2b, these results show that members of the Necl family are differentially expressed by neurons and myelinating Schwann cells in peripheral nerves. While Necl1 and Necl2 are found in sensory neurons, Necl4 is the major Necl that is expressed by myelinating Schwann cells along the nerve.

### Example 3

### The Expression of Necl4 in Schwann Cells is Induced by Axonal Contact and Myelination

To determine the expression of Ned proteins in peripheral nerve, antibodies were raised to Necl1 and Necl4. The specificity of these antibodies was examined using COS-7 cells expressing the different Necl proteins (FIG. 4A). Anti-Necl4 was found to be specific to this Necl and did not recognize Necl1-Necl3, while the anti-Necl1 antibody reacted with Necl1, weakly with Necl2, but not with Necl3 or Necl4. Immunolabeling of cultured rat Schwann cells with these antibodies showed an intense immunoreactivity of Necl4, but not of Necl1 at the cell membrane and processes (FIG. 3). Staining of isolated DRG neurons revealed high levels of Necl1 and weaker levels of Necl4 in the cell soma and along the axons (identified by βIII-tubulin; lower panels in FIG. 3).

While both Necl4 and Necl1 could be immunoprecipitated from rat brain, only Necl4, but not Necl1 was detected in cultured rat Schwann cells (FIG. 4B). Similarly, Necl4 was readily detected by Western blot in lysates of sciatic nerves, further indicating that it is the major member of this family that is found in myelinating glia. These results are in agreement with the Northern blot analysis described in Example 2 (FIG. 2c).

To examine the expression of Necl4 during myelination, we made use of mixed sensory DRG neuron and Schwann cell cultures, which allows a refined analysis of the process (Eshed et al., 2005). Necl4 was weakly expressed in Schwann cells during the first week in culture, but increased thereafter in Schwann cells that were aligned along axons (FIG. 5, a-d). The expression of Necl4 appeared to increase further after the induction of myelination and was particularly high in myelinating Schwann cells labeled for myelin basic protein (MBP) or myelin-associated glycoprotein (MAG) (FIG. 5, e-j). These results demonstrate that Necl4 protein is found in myelinating Schwann cells and indicate that its expression is up-regulated by axonal contact and myelination.

### Example 4

### Complementary Localization of Necl4 and Necl1 Along the Internodes

The localization of Necl4 and Necl1 in myelinated nerves was determined using affinity purified antibodies to these proteins in combination with antibodies to various axonal or glial markers (FIG. 6). In teased sciatic nerve fibers from adult rats, Necl4-immunoreactivity was detected along the internodes but was absent from the nodes (FIG. 6, a-e). This labeling was specific and was completely abolished by preincubating the antibody with a recombinant protein containing the cytoplasmic tail of Necl4 but not of Necl1 (FIG. 6). Double labeling of teased rat sciatic nerve fibers for Necl4 and MAG showed a remarkable co-localization of these proteins in Schmidt-Lanterman incisures (SLI), paranodal loops and all along the adaxonal Schwann cell membrane (apposing the axon) (FIG. 6, f-g). At the paranodes, Necl4-immunoreactivity was distinct from Caspr (FIG. 6e), the latter of which is reported to label the axoglial junction (Poliak and Peles, 2003). In contrast to myelinating Schwann cells, Necl4 was absent from GFAP- and L1-labeled ensheathing non-myelinating Schwann cells (FIG. 8). Immunolabeling of rat sciatic nerve for Necl1 and Na⁺ channels (FIG. 6h), Caspr (FIG. 6i) or Kv1.2 (FIG. 6j), demonstrated that Necl1 was present along the internodes, including the juxtaparanodal region, but was absent from the nodes and paranodes. Occasionally, Necl1 labeling was also detected in the outermost ring of the SLI (FIG. 6k).

Double immunolabeling of cross sections of sciatic nerves for Necl4 and neurofilament (labels axons) or β-dystroglycan (labels abaxonal Schwann cell membrane) provided further evidence that Necl4 was present at the adaxonal membrane surrounding the axon (FIG. 6 i-m). In addition, in ˜15% of the fibers in the sections, Necl4 was found in an inner, wider ring corresponding to incisures (FIG. 6m). In cross sections, Necl1 was localized to the circumference of the axons (marked by neurofilament staining), indicating that it is localized at the axon-Schwann cell interface (FIG. 6 n-o). In summary, these results revealed a complementary localization of Necl4 and Necl1 at the axoglial interface along the internodes.

### Example 5

### Differential Binding of the Extracellular Domain of Necls to Neurons and Schwann Cells

To characterize the interaction between cell adhesion molecules of the Necl family, soluble fusion-proteins of the extracellular domain of each family-member fused to the constant region of human IgG1 were produced (FIG. 9A). We then tested whether these soluble Fc-fusion proteins are able to bind to sensory neurons or Schwann cells. As depicted in FIG. 9B, soluble extracellular domain of Necl1 and Necl3 bound to Schwann cells. In contrast, no binding was detected when using Fc-fusion proteins containing the extracellular domain of Necl2 or Necl4. Similar analysis performed using cultured DRG neurons, revealed that the extracellular domain of all of the examined Necls bound to axons (FIG. 9B). However, while Necl4-Fc robustly labeled the axons, there was a moderate binding of Necl2-Fc, and only weak binding of Necl1-Fc and Necl3-Fc to these cultures. In other experiments using mixed cultures of neurons and glial cells isolated from mice cortex, we found that soluble Necl1 and Necl3 strongly bind to oligodendrocytes, while, the other two Necls bound to axons. These results support a role for Necl proteins in mediating axon-glia interaction in both the peripheral and central nervous system.

### Example 6

### Identification of Necl4 as the Glial Receptor for Axonal Necl1

To determine whether the binding of Necl1-Fc to Schwann cells, as well as the binding of Necl4-Fc to DRG neurons could be mediated by their interaction with other members of the Necl family, we tested the ability Fc-fusion proteins containing the extracellular domains of Necl1-Necl4 to bind COS-7 cells expressing the different Necls. As depicted in FIG. 10A, Necl-Fc molecules were able to bind to COS-7 cells expressing the full-length version of the same molecule (homophilic), as well as to cells expressing other members of the family (heterophilic). In contrast, no binding of soluble Necl-4 was detected to COS-7 cells expressing other immunoglobulin CAMs that were previously shown to mediate axon-glial, such as contactin, neurofascin, TAG-1 and Nr-CAM.

To further determine whether Necl4 is the Schwann cell receptor for axonal Necls, we examined the ability of Necl1-Fc and Necl3-Fc to bind Schwann cells that were transfected with Necl4-specific siRNAs to block its expression (FIG. 10B). In contrast to Schwann cells transfected with a control siRNA, down-regulation of Necl4 in Schwann cells by siRNA abolished the binding of Necl1-Fc and Necl3-Fc. Aggregation of Necl1-Fc on the surface of Schwann cells using a secondary antibody to human Fc, specifically induced the co-clustering of Necl4 but not of other CAMs such as gliomedin, which was clustered by NF155-Fc (FIG. 12B). In a reciprocal set of experiments, reduction of Necl1 expression in DRG neurons by transfection of siRNA abolished the binding of Necl4-Fc (FIG. 11).

Furthermore, aggregation of Necl4-Fc on the surface of DRG neurons induced co-clustering of Necl1 (FIG. 12A). Clustering of Necl1 was specific as Necl4-Fc had no effect on the distribution of other control CAMs such as neurofascin, which was efficiently co-clustered by an Fc-fusion protein containing the olfactomedin-domain of gliomedin (FIG. 12B and Eshed et al., 2005). Taken together, these results demonstrate that Necl4 serves as the glial receptor for axonal Necls, which in the PNS is mainly Necl1.

### Example 7

### Necls Interaction Mediate Schwann Cells Adhesion

To determine whether the interaction between Necl1 and Necl4 is sufficient to mediate axon-glial adhesion, we tested the ability of purified Schwann cells to adhere to an otherwise non-adhesive plastic surface coated with Fc fusion proteins containing the extracellular domain of Necl1 (Necl1-Fc) or Necl4 (Necl4-Fc), using human-Fc and laminin as negative and positive controls, respectively (FIG. 13). Schwann cells adhered to and spread on Necl1-Fc and laminin, but not on Necl4-Fc or human Fc. In contrast to the adherence on laminin, efficient adherence of Schwann cells on Necl1-Fc did not require the presence of Ca²⁺ ions. Pretreating Necl1-Fc substrates with Necl4-Fc, but not with Necl1-Fc, prior to the addition of the Schwann cells, completely abolished their ability to adhere to Necl1-Fc substrate.

### Example 8

### Addition of Necl-Fc to Schwann/Neuron Cultures Inhibit Myelination

The above results suggest that the trans-interaction between Necl4 and Necl1 mediates Schwann cell-axon adhesion. To determine whether disrupting this interaction affects myelination, we added Necl1-Fc, Necl4-Fc, or Zig1-Fc (Zig1 is a related IgCAM expressed in Schwann cells; Spiegel et al., 2006) to co-cultures of DRG neurons and Schwann cells. Fc-fusions were added prior to the induction of myelination, after Schwann cells had already aligned with axons. The cultures were supplemented with fresh Fc-fusion proteins for an additional 10 days and then fixed and immunolabeled for MBP (FIG. 14, a-d). The addition of Necl-Fc fusion proteins resulted in a dramatic reduction (—90% by Necl1-Fc and ±80% by Necl4-Fc, p<0.005) in the number of myelin segments as compared to control-treated (Zig1-Fc) or untreated cultures (FIG. 14e). In contrast, no effect on myelination was observed using soluble extracellular domain of MAG (FIG. 16), indicating that although Necl4 and MAG colocalized in the nerve, they play different roles in PNS myelination. No effect on myelination was also reported for the extracellular domains of other IgCAMs including neurofascin (Eshed et al., 2005; Koticha et al., 2006) and NrCAM (Lustig et al., 2001), further supporting the specificity of the effect of the Necl proteins. Notably, the addition of Necl-Fc fusion proteins to the culture had no effect on Schwann cell proliferation, as determined by the number of DAPI-labeled nuclei per field of view (Necl1-Fc, 478±39; Necl4-Fc, 438±86; Zig1-Fc, 474±40; no Fc, 461±75; n=7), or the percentage of BrdU-incorporated nuclei (Necl1-Fc, 8.3±2.2; Necl4-Fc, 11.2±2.1, Zig1-Fc, 7.4±2.3; no Fc, 10.2±1.9; n=7). These results indicate that disruption of the axon-glia interactions mediated by Necl proteins inhibits myelination.

Electron microscopy analysis of the cultures 9 days after the induction of myelination revealed that many axons were ensheathed by Schwann cells in both the hFc control and the Necl4-Fc-treated cultures (FIG. 15 a-c). However, while in the control cultures 68% (n=87) of the Schwann cell processes wrapped around the axon at least 1.5 turns, in the Necl4-Fc-treated cultures membrane wrapping by Schwann cells was only detected in 12% (n=118) of the cases (FIG. 15 c). Instead, in the Necl4-Fc treated cultures most (88%) of the Schwann cell processes that contacted an axon surrounded it only once or less. In these cultures we frequently detected Schwann cells sending long membrane-protrusions (occasionally sufficient to make a 1.5 turns around the axon), which failed to wrap around the axon, resulting in a horseshoe configuration. This analysis suggests that the Necl proteins are not required for the initial axon-glia contact, but rather for the complete ensheathment by Schwann cells and the transition from the ensheathing stage to myelin wrapping.

### Example 9

### Ectopic Expression of Necl4 Mutants in Schwann Cells Regulates Myelination

To further determine the role of Necl4 in myelination, we expressed a full-length Necl4 cDNA (Necl4-FL), a mutant Necl4 that lacks its entire cytoplasmic domain (Necl4-dCT) or the cytoplasmic domain of Necl4 fused to GFP (GFP-Necl4CT) in Schwann cells before the induction of myelination (FIG. 17A). Necl4-FL and Necl4-dCT were also tagged with a myc-tag sequence in their extracellular domain in order to distinguish them from the endogenous proteins. As a control for GFP-Necl4CT we used a GFP fusion protein containing the cytoplasmic domain of neurofascin (GFP-NF155CT). Immunolabeling of 3T3 fibroblasts infected with retroviruses encoding for the different Necl4 variants used revealed that both Necl4-FL and Necl4-dCT reached the cell surface and efficiently bound Necl1-Fc (FIG. 17C). The expression of GFP-Necl4CT and GFP-NF155CT was demonstrated with antibodies directed against the carboxyl-termini of Necl4 and neurofascin (FIG. 17D).

The same viral stocks were used to infect proliferating Schwann cells in dissociated DRG cultures (Eshed et al., 2005) and myelination was analyzed eighteen days later by staining with an anti-MBP antibody and counting the number of MBP-positive myelin segments. As depicted in FIG. 17E, cultures infected with viruses encoding for Necl4-FL and GFP-Necl4CT virus contain significantly less (˜60-65%; p<0.001) myelinated segments as compared to GFP-infected cultures; expression of GFP-NF155CT had no significant effect on myelination. In addition, no significant difference in the length of the MBP-positive internodes between the cultures infected with the different viruses was found (data not shown). In contrast to cultures expressing Necl4-FL, expression of Necl-dCT resulted in a remarkable increase (˜600%; p<0.001) in the number of myelin segments (FIG. 17F). Taken together, our results demonstrate that an intricate axon-glia contact mediated by Necl proteins is critical for myelination.

### Example 10

### Soluble Necl4 Inhibits Remyelination of Sciatic Nerve

To evaluate the significance of Necl1-Necl4 interactions during myelination in vivo, we made use of a PNS remyelination paradigm (Hall and Gregson, 1971). In this experimental setting, demyelination is induced by lysolecithin, and is followed by a period of remyelination, which is fundamentally similar to developmental myelination. This model has the advantage that it allows an examination of the effect on myelination of various substances introduced directly into the nerve. Furthermore, it avoids the technical difficulties of working with premyelinated axons of newborn animals due to the smaller size of their nerves, and the fact that the perineurial barrier has not yet fully formed (Thomas and Olsson, 1984). We induced a focal demyelinating lesion of sciatic nerves by intraneural injection of lysolecithin and then injected either Necl4-Fc or hFc fusion proteins directly into the demyelinated site 5 and again at 8 days later, at a time when Schwann cells actively remyelinate the lesion (Dugandzija-Novakovic et al., 1995). If Necl4 is indeed required for myelination, then addition of the Necl4-Fc fusion protein should function to inhibit remyelination in this model by competing with endogenous Schwann cell Necl4 for the binding of axonal Necl1. We examined the injection sites at 11 dpi by immunofluorescence labeling for MBP, as a marker for compact myelin and for Na⁺ channels, as a marker for nodes of Ranvier. Na⁺ channel clustering was used since after remyelination, new myelin segments are shorter, resulting in an increased number of nodes of Ranvier (Dugandzija-Novakovic et al., 1995). In both the Necl4-Fc and hFc-injected nerves, we found unaffected regions that had intact myelin sheaths with relatively few nodes of Ranvier (FIG. 18a, region c), as well as remyelinated nerve fibers (FIG. 18a, region f and FIG. 18b, region d). Remyelinated regions were clearly identified by reduced MBP immunoreactivity and the frequent occurrence of binary clusters of Na⁺ channels (FIG. 18 a-b, inset). However, in contrast to the control hFc-injected nerves, in the Necl4-Fc injected nerves we also observed large areas that had relatively few remyelinated axons (FIG. 18b, region e, and FIG. 18e). Compared to the remyelinated regions (FIG. 18 d,f), these zones had 2-3 fold fewer Na⁺ channel clusters (FIG. 18 e, g), further indicating that the presence of a soluble extracellular domain of Necl4 in the demyelinated nerve inhibits remyelination.

### CONCLUSION

The results shown herein provide evidence that Necl4 (SynCAM4) and Necl1 (SynCAM3) mediate critical interactions between Schwann cells and axons during myelination: (i) These two CAMs are expressed on apposed cell membranes along the internode of myelinated axons, i.e., Schwann cells express Necl4 and axons express Necl1; (ii) Axonal contact increases the expression of Necl4 in Schwann cells, especially at the onset of myelination; (iii) Necl4 is the glial binding partner of axonal Necl1, and is both necessary and sufficient for Necl1 binding; (iv) Necl4 and Necl1 can recruit and cluster each other specifically to sites of higher ligand concentration on axons and Schwann cells respectively; (v) The interaction between Necl1 and Necl4 mediates Schwann cell adhesion; (vi) Interfering with Necl4Necl1 interaction in myelinated cultures using a soluble ectodomain of either molecule inhibits myelination; (vii) Ectopic expression of a dominant negative mutant of Necl4 in Schwann cells markedly reduces myelination, and (viii) Intraneural injection of a soluble Necl4 protein inhibited remyelination of demyelinated sciatic nerves. Based on these results, Necl4 and Necl1 appear to mediate Schwann cell-axon interaction(s) required for myelination.

### REFERENCES

Adamsky, K., Schilling, J., Garwood, J., Faissner, A., and Peles, E. (2001). Glial tumor cell adhesion is mediated by binding of the FNIII domain of receptor protein tyrosine phosphatase beta (RPTPbeta) to tenascin C. Oncogene 20, 609-618.
Altschul, S. F., Gish, W., Miller, W., Myers, E. W. & Lipman, D. J. (1990) Basic local alignment search tool. J. Mol. Biol. 215, 403-410.
Biederer, T., Sara, Y., Mozhayeva, M., Atasoy, D., Liu, X., Kavalali, E. T., and Sudhof, T. C. (2002). SynCAM, a synaptic adhesion molecule that drives synapse assembly. Science 297, 1525-1531.
Dugandzija-Novakovic, S., Koszowski, A. G., Levinson, S. R. & Shrager, P. (1995). Clustering of Na+channels and node of Ranvier formation in remyelinating axons. J. Neurosci. 15, 492-503 Eshed, Y., Feinberg, K., Poliak, S., Sabanay, H., Sarig-Nadir, O., Spiegel, I., Bermingham, J. R., Jr., and Peles, E. (2005). Gliomedin mediates Schwann cell-axon interaction and the molecular assembly of the nodes of Ranvier. Neuron 47, 215-229.
Fire, A., Uu, S., Montgomery, M. K., Kostas, S. A., Driver, S. e. and Mello, C. C. (1998) Potent and specific genetic interference by double-stranded RNA in Caenorhabditis elegans. Nature 391, 806-811.
Friede, R. L. and Samorajski, T. (1968). Myelin formation in the sciatic nerve of the rat. A quantitative electron microscopic, histochemical and radioautographic study. J Neuropath Exp Neurol 27, 546-570.
Gollan, L., Salomon, D., Salzer, J. L., and Peles, E. (2003). Caspr regulates the processing of contactin and inhibits its binding to neurofascin. J Cell Biol 163, 1213-1218.
Grishok, A., Pasquinelli, A. E., conte, D., Li, N., Parrish, S., Ha, I., Baillie, D. L., fire, A., Ruvkun, G. and Mello, C. C. (2001). Genes and mechanisms related to RNA interference regulate expression of the small temporal RNAs that control C. elegans developmental timing. Cell 106, 23-34.
Hall, S. M. and Gregson, N. A. (1971). The in vivo and ultrastructural effects of injection of lysophosphatidyl choline into myelinated peripheral nerve fibres of the adult mouse. J. Cell Sci. 9, 769-89.
Jessen, K. R., and Mirsky, R. (2005). The origin and development of glial cells in peripheral nerves. Nat Rev Neurosci 6, 671-682.
Kakunaga, S., Ikeda, W., Itoh, S., Deguchi-Tawarada, M., Ohtsuka, T., Mizoguchi, A., and Takai, Y. (2005). Nectin-like molecule-1/TSLL1/SynCAM3: a neural tissue-specific immunoglobulin-like cell-cell adhesion molecule localizing at non junctional contact sites of presynaptic nerve terminals, axons and glia cell processes. J Cell Sci 118, 1267-1277.
Ketting, R. F., Haverkamp, T. H., van Luenen, H. G. and Plasterk, R. H. (2001). Mut-7 of C. elegans, required for transposon silencing and RNA interference, is a homolog of Werner syndrome helicase and RNaseD. Cell 99, 133-141.
Kojima, T., and Kitamura, T. (1999). A signal sequence trap based on a constitutively active cytokine receptor. Nat Biotechnol 17, 487-490.
Koroll, M., Rathjen, F. G., and Volkmer, H. (2001). The neural cell recognition molecule neurofascin interacts with syntenin-1 but not with syntenin-2, both of which reveal self-associating activity. J Biol Chem 276, 10646-10654.
Kuramochi, M., Fukuhara, H., Nobukuni, T., Kanbe, T., Maruyama, T., Ghosh, H. P., Pletcher, M., Isomura, M., Onizuka, M., Kitamura, T., et al. (2001). TSLC1 is a tumor-suppressor gene in human non-small-cell lung cancer. Nat Genet 27, 427-430.
Koticha, D., Maurel, P., Zanazzi, G., Kane-Goldsmith, N., Basak, S., Bablarz, J., Salzer, J. and Grumet, M. (2006). Neurofascin interactions play a critical role in clustering sodium channels, ankyrin G and beta IV spectrin at peripheral nodes of Ranvier. Dev. Biol. 293, 1-12. Lin, R. and Avery, L. (1999). RNA interference. Policing rogue genes. Nature 402, 128-129.
Lustig, M., Zanazzi, G., Sakurai, T., Blanco, C., Levinson, S. R., Lamber, S., Grumet, M. and Salzer, J. L. (2001). Nr-CAM and neurofascin interactions regulate ankyrin G and sodium channel clustering at the node of Ranvier. Curr. Biol. 11, 1864-9. Mao, X., Seidlitz, E., Ghosh, K., Murakami, Y., and Ghosh, H. P. (2003). The cytoplasmic domain is critical to the tumor suppressor activity of TSLC1 in non-small cell lung cancer. Cancer Res 63, 7979-7985.
Montgomery, M. K., Xu, S. and Fire, A. (1998) RNA as a target of double-stranded RNA-mediated genetic interference in Caenorhabditis elegans. Proc. Natl. Acad. Sci. USA, 95, 155-2-15507.
Morgan, L. Jessen, K. R., and Mirsky, R. (1991) The effects of camp on differentiation of cutured Schwann cells: pregression from an early pheotype (04+) to a myelin phenotype (PO+, GFAP-, N-CAM-, NGF-receptor-) depends on growth inhibition. J. Cell Biol. 112, 457-467.
Pedraza, L., and Colman, D. R. (2000). Fluorescent myelin proteins provide new tools to study the myelination process. J Neurosci Res 60, 697-703.
Peles, E., Nativ, M., Lustig, M., Grumet, M., Schilling, J., Martinez, R., Plowman, G. D., and Schlessinger, J. (1997). Identification of a novel contactin-associated transmembrane receptor with multiple domains implicated in protein-protein interactions. Embo J 16, 978-988.
Poliak, S. and Peles, E. (2003). The local differentiation of myelinated axons at nodes of Ranvier. Nat Rev Neurosci 4, 968-80.
Poliak, S., Gollan, L., Martinez, R., Custer, A., Einheber, S., Salzer, J. L., Trimmer, J. S., Shrager, P., and Peles, E. (1999). Caspr2, a new member of the neurexin superfamily, is localized at the juxtaparanodes of myelinated axons and associates with K+ channels. Neuron 24, 1037-1047.
Poliak, S., Matlis, S., Ullmer, C., Scherer, S. S., and Peles, E. (2002). Distinct claudins and associated PDZ proteins form different autotypic tight junctions in myelinating Schwann cells. J Cell Biol 159, 361-372.
Poliak, S., Salomon, D., Elhanany, H., Sabanay, H., Kiernan, B., Pevny, L., Stewart, C. L., Xu, X., Chiu, S. Y., Shrager, P., et al. (2003). Juxtaparanodal clustering of Shaker-like K+ channels in myelinated axons depends on Caspr2 and TAG-1. J Cell Biol 162, 1149-1160.
Sambrook et al., (2001Molecular Cloning, Cold Spring Harbor Laboratory Press, Cold Spring Harbor, N.Y.
Schafer, D. P., Bansal, R., Hedstrom, K. L., Pfeiffer, S. E. and Rasband, M. N. (2004). Does paranode formation and maintenance require partitioning of neurofascin 155 into lipid rafts? J. Neurosci. 24, 3176-85.
Shingai, T., Ikeda, W., Kakunaga, S., Morimoto, K., Takekuni, K., Itoh, S., Satoh, K., Takeuchi, M., Imai, T., Monden, M., and Takai, Y. (2003). Implications of nectin-like molecule-2/IGSF4/RA175/SgIGSF/TSLC1/SynCAM1 in cell-cell adhesion and transmembrane protein localization in epithelial cells. J Biol Chem 278, 35421-35427.
Spiegel, I., Adamsky, K., Eisenbach, M., Eshed, Y., Spiegel, A., Mirsky, R., Scherer, S. S., and Peles, E. (2006). Identification of novel cell-adhesion molecules in peripheral nerves using a signal-sequence trap. Neuron Glia Biology 2, 27-38.
Spiegel, I., Salomon, D., Erne, B., Schaeren-Wiemers, N., and Peles, E. (2002). Caspr3 and caspr4, two novel members of the caspr family are expressed in the nervous system and interact with PDZ domains. Mol Cell Neurosci 20, 283-297. Tabara, H., Sarkissian, M., Kelly, W. G., Fleenor, J., Grishok, A., Timmons, L., Fire, A. and Mello, C. C. (1999). The rde-1 gene, RNA interference, and transposon silencing in C. elegans. Cell, 99, 123-132.
Thomas, P. K. and Olsson, Y. (1984) Microscopic anatomy and function of the connective tissue components of peripheral nerve., pages 97-120, in Peripheral Neuropathy, eds. Dyck, P. J., Thomas, P. K., Lambert, E. H. & Bunge, R., W. B. Saunders., Philadelphia.
Williams, Y. N., Masuda, M., Sakurai-Yageta, M., Maruyama, T., Shibuya, M., and Murakami, Y. (2006). Cell adhesion and prostate tumor-suppressor activity of TSLL2/IGSF4C, an immunoglobulin superfamily molecule homologous to TSLC1/IGSF4. Oncogene 25(10):1446-53.
Yageta, M., Kuramochi, M., Masuda, M., Fukami, T., Fukuhara, H., Maruyama, T., Shibuya, M., and Murakami, Y. (2002). Direct association of TSLC1 and DAL-1, two distinct tumor suppressor proteins in lung cancer. Cancer Res 62, 5129-5133.
Zhou, Y., Du, G., Hu, X., Yu, S., Liu, Y., Xu, Y., Huang, X., Liu, J., Yin, B., Fan, M., et al. (2005). Nectin-like molecule 1 is a protein 4.1N associated protein and recruits protein 4.1N from cytoplasm to the plasma membrane. Biochim Biophys Acta 1669, 142-154.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-55. (canceled)

56. A polypeptide comprising an isolated domain of human Necl4, or a fragment, analog or derivative thereof, wherein the domain of human Necl4 is selected from the group consisting of:
i) a cytoplasmic domain wherein the polypeptide lacks at least one domain of human Necl4 selected from the group consisting of: an extracellular domain and a transmembrane domain; and
ii) the extracellular domain, wherein the polypeptide lacks at least one domain of human Necl4 selected from the group consisting of: the cytoplasmic domain and the transmembrane domain.

57. The polypeptide of claim 56, comprising a domain selected from the group consisting of:
the cytoplasmic domain of human Necl4 as set forth in a sequence selected from the group consisting of SEQ ID NO:4 and SEQ ID NO:8;
the extracellular domain of human Necl4 as set forth in a sequence selected from the group consisting of SEQ ID NO:2; SEQ ID NO:3; SEQ ID NO:6; SEQ ID NO:7; SEQ ID NO:10; SEQ ID NO:11; SEQ ID NO:98 and SEQ ID NO:99; and
further optionally comprising the transmembrane domain of human Necl4 as set forth in SEQ ID NO:5.

58. The polypeptide of claim 56, wherein the polypeptide is a fusion protein further comprising an amino acid sequence derived from a heterologous protein, wherein the heterologous protein is selected from the group consisting of: an immunoglobulin; a marker protein; a protein associated with neural cells; a heterologous human Necl, and a fragment thereof.

59. The polypeptide of claim 58, wherein the heterologous protein is an immunoglobulin fragment selected from the group consisting of: Fc; Fab; scFv; dsFv; V_{L} and V_{H}.

60. The polypeptide of claim 59, wherein the isolated domain of human Necl4 is the extracellular domain selected from the group consisting of SEQ ID NO:2; SEQ ID NO:3; SEQ ID NO:6; SEQ ID NO:7; SEQ ID NO:10; SEQ ID NO:11; SEQ ID NO:98 and SEQ ID NO:99, and the immunoglobulin fragment is Fc.

61. The polypeptide of claim 58, wherein the heterologous protein is selected from the group consisting of:
an immunoglobulin or fragment thereof having specificity for a protein associated with neural cells, wherein the protein associated with neural cells is selected from the group consisting of MBP; MAG; Caspr; GFAP; L1; contactin; neurofascin; TAG-1; Nr-CAM and gliomedin;
a protein associated with neural cells, selected from the group consisting of MBP; MAG; Caspr; GFAP; L1; contactin; neurofascin; TAG-1; Nr-CAM and gliomedin; and
an isolated domain of a heterologous human Necl, selected from the group consisting of: SEQ ID NO:17; SEQ ID NO:18; SEQ ID NO:19; SEQ ID NO:20; SEQ ID NO:21; SEQ ID NO:22; SEQ ID NO:23; SEQ ID NO:24, and SEQ ID NO:25.

62. The polypeptide of claim 56, comprising a chemical modification selected from the group consisting of: glycosylation, pegylation, oxidation, permanent phosphorylation, reduction, myristylation, sulfation, acylation, acetylation, ADP-ribosylation, amidation, hydroxylation, iodination, methylation, and derivatization by blocking groups.

63. A pharmaceutical composition comprising as an active ingredient the polypeptide of claim 57.

64. The pharmaceutical composition of claim 63, wherein the polypeptide is a fusion protein further comprising an amino acid sequence derived from a heterologous protein, wherein the heterologous protein is selected from the group consisting of: an immunoglobulin; a marker protein; a protein associated with neural cells; an isolated domain of a heterologous human Ned, and a fragment thereof.

65. The pharmaceutical composition of claim 64, wherein the protein associated with neural cells is selected from the group consisting of MBP; MAG; Caspr; GFAP; L1; contactin; neurofascin; TAG-1; Nr-CAM and gliomedin.

66. An isolated polynucleotide sequence encoding the polypeptide of claim 57.

67. The isolated polynucleotide sequence of claim 66, comprising a nucleotide sequence selected from the group consisting of: SEQ ID NO:50; SEQ ID NO:51; SEQ ID NO:52; SEQ ID NO:53 and SEQ ID NO:103.

68. An expression vector comprising the isolated polynucleotide of claim 66, and further comprising at least one regulatory element operatively linked to the polynucleotide, wherein the regulatory element is selected from the group consisting of: a promoter, an enhancer, a selectable gene, a signal peptide, a recombinase gene, a transcription factor gene and a reporter gene.

69. The expression vector of claim 68, having a nucleotide sequence selected from the group consisting of: SEQ ID NO:89 and SEQ ID NO:91.

70. A pharmaceutical composition comprising as an active ingredient the expression vector of claim 68, and a pharmaceutically acceptable carrier.

71. A host cell transformed with the expression vector of claim 68, wherein the host cell is selected from the group consisting of: an eucaryotic cell; a somatic cell; a germ cell; a neuronal cell; a pluripotent stem cell; and a nerve progenitor cell.

72. The host cell of claim 71, wherein the neuronal cell is selected from the group consisting of: Schwann cell; myelinating Schwann cell; glial cell; and dorsal root ganglion neuron.

73. A method of treating neurological damage, wherein the method comprises administering to a subject in need thereof a pharmaceutical composition comprising an expression vector comprising an isolated polynucleotide sequence, wherein the isolated polynucleotide sequence encodes the polypeptide of claim 57, thereby treating the neurological damage.

74. The method of claim 73, wherein the neurological damage is selected from the group consisting of:
neurological damage comprising nerve demyelination associated with the peripheral nervous system or the central nervous system;
neurological damage associated with a neural injury or disease, wherein the disease is selected from the group consisting of: acute inflammatory demyelinating polyradiculoneuropathy, chronic inflammatory demyelinating polyradiculoneuropathy, chronic idiopathic axonal polyneuropathy, adrenoleukodystrophy, diabetic neuropathy, Guillain-Barre disease (acute demyelinating polyneuropathy), multiple sclerosis, HIV inflammatory demyelinating disease and post infection encephalomyelitis.

75. An siRNA molecule capable of down regulating expression of Necl4 via RNA interference, wherein the siRNA molecule is selected from the group consisting of: a double stranded molecule comprising two separate RNA strands in which one strand has at least one region complementary to a region on the other strand and a single stranded molecule comprising a hairpin loop wherein at least one region of the hairpin loop is complementary to an opposing region of the hairpin loop.

76. The siRNA molecule of claim 75, wherein the siRNA comprises an oligonucleotide selected from the group consisting of SEQ ID NO:55 and SEQ ID NO:56.

77. A method of down regulating Necl4 expression in a cell, comprising contacting the cell with the siRNA molecule of claim 76, under conditions suitable for down regulating of Necl4 expression.

78. A method of inhibiting Necl4 activity in a cell, the method comprising down regulating expression of Necl1 via RNA interference with an siRNA molecule specific for Necl1.

79. The method of claim 78, wherein the siRNA molecule specific for Necl1 comprises the oligonucleotide of SEQ ID NO:57.


SEQUENCE LISTING (104 sequences)

SEQ ID NO: 1 - protein, 388 aa, Homo sapiens
MGRARRFQWPLLLLWAAAAGPGAGQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAATLRWYRDRKELKGVSSSQENGKVWSVASTVRFRVDRKDDGGIIICEAQNQALPSGHSKQTQYVLDVQYSPTARIHASQAVVREGDTLVLTCAVTGNPRPNQIRWNRGNESLPERAEAVGETLTLPGLVSADNGTYTCEASNKHGHARALYVLVVYDPGAVVEAQTSVPYAIVGGILALLVFLIICVLVGMVWCSVRQKGSYLTHEASGLDEQGEAREAFLNGSDGHKRKEEFFI

SEQ ID NO: 2 - protein, 323 aa, Homo sapiens
MGRARRFQWPLLLLWAAAAGPGAGQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAATLRWYRDRKELKGVSSSQENGKVWSVASTVRFRVDRKDDGGIIICEAQNQALPSGHSKQTQYVLDVQYSPTARIHASQAVVREGDTLVLTCAVTGNPRPNQIRWNRGNESLPERAEAVGETLTLPGLVSADNGTYTCEASNKHGHARALYVLVVYDPGAVVEAQTSVPY

SEQ ID NO: 3 - protein, 299 aa, Homo sapiens
QEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAATLRWYRDRKELKGVSSSQENGKVWSVASTVRFRVDRKDDGGIIICEAQNQALPSGHSKQTQYVLDVQYSPTARIHASQAVVREGDTLVLTCAVTGNPRPNQIRWNRGNESLPERAEAVGETLTLPGLVSADNGTYTCEASNKHGHARALYVLVVYDPGAVVEAQTSVPY

SEQ ID NO: 4 - protein, 42 aa, Homo sapiens
CSVRQKGSYLTHEASGLDEQGEAREAFLNGSDGHKRKEEFFI

SEQ ID NO: 5 - protein, 23 aa, Homo sapiens
AIVGGILALLVFLIICVLVGMVW

SEQ ID NO: 6 - protein, 371 aa, Rattus norvegicus
MTHRGAEVNEEPMGLLRRRLLLLVVVVTTCVPASQGLRCIQCESNQDCLVEECALGQDLCRTTVLREWEGTAQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAAVLRWYRDRKELKGVSSGQENGKVWSVASTVRFRVDRKDDGGIVICEAQNQALPSGHSKQTQYVLDVQYSPTARIHASQAVVREGDTLVLTCAVTGNPRPNQIRWNRGNESLPERAEALGETLTLPGLVSADNGTYTCEAANKHGHARALYVLVVYDPGAVVEAQTSVPY

SEQ ID NO: 7 - protein, 349 aa, Rattus norvegicus
LVVVVTTCVPASQGLRCIQCESNQDCLVEECALGQDLCRTTVLREWEGTAQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAAVLRWYRDRKELKGVSSGQENGKVWSVASTVRFRVDRKDDGGIVICEAQNQALPSGHSKQTQYVLDVQYSPTARIHASQAVVREGDTLVLTCAVTGNPRPNQIRWNRGNESLPERAEALGETLTLPGLVSADNGTYTCEAANKHGHARALYVLVVYDPGAVVEAQTSVPY

SEQ ID NO: 8 - protein, 42 aa, Rattus norvegicus; identical to SEQ ID NO: 4

SEQ ID NO: 9 - protein, 23 aa, Rattus norvegicus
AIVGGILALLVFLIICVLVGMVW

SEQ ID NO: 10 - protein, 323 aa, Mus musculus
MGRARRFQWPLLLLWAAAAGPGTGQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAAVLRWYRDRKELKGVSSGQENGKVWSVASTVRFRVDRKDDGGIVICEAQNQALPSGHSKQTQYVLDVQYSPTARIHASQAVVREGDTLVLTCAVTGNPRPNQIRWNRGNESLPERAEAVGETLTLPGLVSADNGTYTCEAANKHGHARALYVLVVYDPGAVVEAQTSVPY

SEQ ID NO: 11 - protein, 301 aa, Mus musculus
TGQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAAVLRWYRDRKELKGVSSGQENGKVWSVASTVRFRVDRKDDGGIVICEAQNQALPSGHSKQTQYVLDVQYSPTARIHASQAVVREGDTLVLTCAVTGNPRPNQIRWNRGNESLPERAEAVGETLTLPGLVSADNGTYTCEAANKHGHARALYVLVVYDPGAVVEAQTSVPY

SEQ ID NO: 12 - protein, 42 aa, Mus musculus
CSVRQKGSYLTHEASGLDEQGEAREAFLNGGDGHKRKEEFFI

SEQ ID NO: 13 - protein, 23 aa, Mus musculus
AIVGGILALLVFLIICVLVGMVW

SEQ ID NO: 14 - protein, 24 aa, Homo sapiens
MGRARRFQWPLLLLWAAAAGPGAG

SEQ ID NO: 15 - protein, 22 aa, Rattus norvegicus
MTHRGAEVNEEPMGLLRRRLLL

SEQ ID NO: 16 - protein, 22 aa, Mus musculus
MGRARRFQWPLLLLWAAAAGPG

SEQ ID NO: 17 - protein, 363 aa, Homo sapiens
MGAPAASLLLLLLLFACCWAPGGANLSQDGYWQEQDLELGTLAPLDEAISSTVWSSPDMLASQDSQPWTSDETVVAGGTVVLKCQVKDHEDSSLQWSNPAQQTLYFGEKRALRDNRIQLVTSTPHELSISISNVALADEGEYTCSIFTMPVRTAKSLVTVLGIPQKPIITGYKSSLREKDTATLNCQSSGSKPAARLTWRKGDQELHGEPTRIQEDPNGKTFTVSSSVTFQVTREDDGASIVCSVNHESLKGADRSTSQRIEVLYTPTAMIRPDPPHPREGQKLLLHCEGRGNPVPQQYLWEKEGSVPPLKMTQESALIFPFLNKSDSGTYGCTATSNMGSYKAYYTLNVNDPSPVPSSSSTY

SEQ ID NO: 18 - protein, 46 aa, Homo sapiens
HYLIRHKGTYLTHEAKGSDDAPDADTAIINAEGGQSGGDDKKEYFI

SEQ ID NO: 19 - protein, 23 aa, Homo sapiens
HAIIGGIVAFIVFLLLIMLIFLG

SEQ ID NO: 20 - protein, 373 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQRDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVD

SEQ ID NO: 21 - protein, 46 aa, Homo sapiens
RYFARHKGTYFTHEAKGADDAADADTAIINAEGGQNNSEEKKEYFI

SEQ ID NO: 22 - protein, 23 aa, Homo sapiens
HAVIGGVVAVVVFAMLCLLIILG

SEQ ID NO: 23 - protein, 368 aa, Homo sapiens
MFVLFLCNLSLVPAAASKNKVKGSQGQFPLTQNVTVVEGGTAILTCRVDQNDNTSLQWSNPAQQTLYFDDKKALRDNRIELVRASWHELSISVSDVSLSDEGQYTCSLFTMPVKTSKAYLTVLGVPEKPQISGFSSPVMEGDLMQLTCKTSGSKPAADIRWFKNDKEIKDVKYLKEEDANRKTFTVSSTLDFRVDRSDDGVAVICRVDHESLNATPQVAMQVLEIHYTPSVKIIPSTPFPQEGQPLILTCESKGKPLPEPVLWTKDGGELPDPDRMVVSGRELNILFLNKTDNGTYRCEATNTIGQSSAEYVLIVHDVPNTLLPTTIIPSLTTATVTTTVAITTSPTTSATTSSIRDPNALAGQNGPD

SEQ ID NO: 24 - protein, 46 aa, Homo sapiens
RYLARHKGTYLTNEAKGAEDAPDADTAIINAEGSQVNAEEKKEYFI

SEQ ID NO: 25 - protein, 23 aa, Homo sapiens
HALIGGIVAVVVFVTLCSIFLLG

SEQ ID NO: 26 - protein, 360 aa, Rattus norvegicus
MGAPSALPLLLLLACSWAPGGANLSQDGYWQEQDLELGTPAPLEEALSSTVWSNPDLLASQDSQPWTSDETVVAGGTVVLKCQVKDHEDSSLQWSNPAQQTLYFGEKRALRDNRIQLVSSTPHELSISISNVALADEGEYTCSIFTMPVRTAKSLVTVLGIPQKPIITGYKSSLREKETATLNCQSSGSKPAAQLAWRKGDQELHGDQTRIQEDPNGKTFTVSSSVSFQVTRDDDGANVVCSVNHESLKGADRSTSQRIEVLYTPTAMIRPEPAHPREGQKLLLHCEGRGNPVPQQYVWVKEGSEPPLKMTQESALIFPFLNKSDSGTYGCTATSNMGSYTAYFTLNVNDPSPVPSSSST

SEQ ID NO: 27 - protein, 47 aa, Rattus norvegicus
GHYLIRHKGTYLTHEAKGSDDAPDADTAIINAEGGQSGGDDKKEYFI

SEQ ID NO: 28 - protein, 23 aa, Rattus norvegicus
YHAIIGGIVAFIVFLLLILLIFL

SEQ ID NO: 29 - protein, 407 aa, Rattus norvegicus
MASPVLPSGSQCAAAAAVAAAAAPPGLRLRLLLLLLSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNKELKGKSEVEEWSDMYTVTSQLMLKVHKEDDGVPVICQVEHPAVTGNLQTQRYLEVQYKPQVQIQMTYPLQGLTREGDAFELTCEATGKPQPVMVTWVRVDDEMPQHAVLSGPNLFINNLNKTDNGTYRCEASNTVGKAHSDYMLYVYDPPTTIPPPTTTTTTTTTTTTTTTILTIITDTTATTEPAVHGLTQLPNSAEELDSEDLSDSRAGEEGAIGAVD

SEQ ID NO: 30 - protein, 46 aa, Rattus norvegicus; identical to SEQ ID NO: 21

SEQ ID NO: 31 - protein, 23 aa, Rattus norvegicus
HAVIGGVVAVVVFAMLCLLIILG

SEQ ID NO: 32 - protein, 393 aa, Rattus norvegicus
MEASIYSAPPCSASGAEEFSPAGSSFFAICGLRVLRASVSKPTLRWLLTLSLISLIFSVLFIAILSRLSLVWRTPAECAAASKSKVKGSQGQFPLTQNVTVVEGGTAILTCRVDQNDNTSLQWSNPAQQTLYFDDKKALRDNRIELVRASWHELSISVSDVSLSDEGQYTCSLFTMPVKTSKAYLTVLGVPEKPQISGFSSPVMEGDLMQLTCKTSGSKPAADIRWFKNDKEIKDVKYLKEEDANRKTFTVSSTLDFRVDRSDDGVAVICRVDHESLNATPQVAMQVLEIHYTPSVKIIPSTPFPQEGQALTLTCESKGKPLPEPVLWTKDGAELPDPDRMVVSGRELNILFLNKTDNGTYRCEATNTIGQSSAEYVLIVHDPNSLAGQNGPD

SEQ ID NO: 33 - protein, 46 aa, Rattus norvegicus; identical to SEQ ID NO: 24

SEQ ID NO: 34 - protein, 23 aa, Rattus norvegicus
HALIGGIVAVVVFVTLCSIFLLG

SEQ ID NO: 35 - protein, 326 aa, Mus musculus
MGAPSALPLLLLLACSWAPGGANLSQDDSQPWTSDETVVAGGTVVLKCQVKDHEDSSLQWSNPAQQTLYFGEKRALRDNRIQLVSSTPHELSISISNVALADEGEYTCSIFTMPVRTAKSLVTVLGIPQKPIITGYKSSLREKETATLNCQSSGSKPAAQLTWRKGDQELHGDQTRIQEDPNGKTFTVSSSVSFQVTREDDGANIVCSVNHESLKGADRSTSQRIEVLYTPTAMIRPEPAHPREGQKLLLHCEGRGNPVPQQYVWVKEGSEPPLKMTQESALIFPFLNKSDSGTYGCTATSNMGSYTAYFTLNVNDPSPVPSSSST

SEQ ID NO: 36 - protein, 47 aa, Mus musculus; identical to SEQ ID NO: 27

SEQ ID NO: 37 - protein, 23 aa, Mus musculus
YHAIIGGIVAFIVFLLLILLIFL

SEQ ID NO: 38 - protein, 387 aa, Mus musculus
MASAVLPSGSQCAAAAAVAAAAAPPGLRLRLLLLLLSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNKELKGKSEVEEWSDMYTVTSQLMLKVHKEDDGVPVICQVEHPAVTGNLQTQRYLEVQYKPQVHIQMTYPLQGLTREGDAFELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDTTATTEPAVHDSRAGEEGTIGAVD

SEQ ID NO: 39 - protein, 46 aa, Mus musculus; identical to SEQ ID NO: 21

SEQ ID NO: 40 - protein, 23 aa, Mus musculus
HAVIGGVVAVVVFAMLCLLIILG

SEQ ID NO: 41 - protein, 335 aa, Mus musculus
MIWKRSAVLRFYSVCGLLLQAAASKSKVKGSQGQFPLTQNVTVVEGGTAILTCRVDQNDNTSLQWSNPAQQTLYFDDKKALRDNRIELVRASWHELSISVSDVSLSDEGQYTCSLFTMPVKTSKAYLTVLGVPEKPQISGFSSPVMEGDLMQLTCKTSGSKPAADIRWFKNDKEIKDVKYLKEEDANRKTFTVSSTLDFRVDRSDDGVAVICRVDHESLNATPQVAMQVLEIHYTPSVKIIPSTPFPQEGQALTLTCESKGKPLPEPVLWTKDGAELPDPDRMVVSGRELNILFLNKTDNGTYRCKATNTIGQSSAEYVLIVHDPNSLAGQNGPD

SEQ ID NO: 42 - protein, 46 aa, Mus musculus; identical to SEQ ID NO: 24

SEQ ID NO: 43 - protein, 23 aa, Mus musculus
HALIGGIVAVVVFVTLCSIFLLG

SEQ ID NO: 44 - protein, 232 aa, Artificial Sequence
  Synthetic
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 239 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 46 - protein, 409 aa, Artificial Sequence
  Synthetic
METDTLLLWVLLLWVPGSTGDAAQPARRAVRSLDPEQKLISEEDLSMMQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAAVLRWYRDRKELKGVSSGQENGKVWSVASTVRFRVDRKDDGGIVICEAQNQALPSGHSKQTQYVLDVQYSPTARIHASQAVVREGDTLVLTCAVTGNPRPNQIRWNRGNESLPERAEAVGETLTLPGLVSADNGTYTCEAANKHGHARALYVLVVYDPGAVVEAQTSVPYAIVGGILALLVFLIICVLVGMVWCSVRQKGSYLTHEASGLDEQGEAREAFLNGGDGHKRKEEFFI

SEQ ID NO: 47 - protein, 365 aa, Artificial Sequence
  Synthetic
METDTLLLWVLLLWVPGSTGDAAQPARRAVRSLDPEQKLISEEDLSMQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAAVLRWYRDRKELKGVSSGQENGKVWSVASTVRFRVDRKDDGGIVICEAQNQALPSGHSKQTQYVLDVQYSPTARIHASQAVVREGDTLVLTCAVTGNPRPNQIRWNRGNESLPERAEAVGETLTLPGLVSADNGTYTCEAANKHGHARALYVLVVYDPGAVVEAQTSVPYAIVGGILALLVFLIICVLVGMV

SEQ ID NO: 48 - protein, 20 aa, Artificial Sequence
  Synthetic
METDTLLLWVLLLWVPGSTG

SEQ ID NO: 49 - protein, 10 aa, Artificial Sequence
  Synthetic
EQKLISEEDL

SEQ ID NO: 50 - DNA, 894 bp, Artificial Sequence
  Synthetic
atgggccgggcccggcgcttccagtggccgctgctgctgctgtgggcggccgcggcggggccaggggcaggacaggaagtacagacagagaacgtgacagtggctgagggtggggtggctgagatcacctgccgtctgcaccagtatgatgggtccatagttgtcatccagaacccagcccggcagaccctcttcttcaatggcacccgtgccttgaaggatgagcgtttccagcttgaggagttctccccacgccgggtgcggatccggctctcagatgcccgcctggaggacgaggggggctatttctgccagctctacacagaagacacccaccaccagattgccacgctcacggtactagtggccccagagaatcctgtggtggaggtccgggagcaggcggtagagggcggcgaggtggagctcagctgcctcgttccgcggtcccgtccggctgccaccctgcgctggtaccgggaccgcaaggagctgaaaggagtgagcagcagccaggaaaatggcaaggtctggagcgtggcaagcacagtacggtttcgtgtggaccgtaaggacgacggtggtatcatcatctgtgaggcgcagaaccaggcgctgccctccggacacagcaagcagacgcagtacgtgctggatgtgcagtactcccccacggcccggattcatgcctcccaagctgtggtgagggagggagacacgctggtgttgacgtgtgctgtcacggggaaccccaggccaaaccagatccgctggaaccgcgggaatgagtctttgccggagagggcggaggccgtgggagagacgctcacgctgccgggtctggtatccgcggataacggcacctacacttgcgaggcgtccaataagcacggc

SEQ ID NO: 51 - DNA, 822 bp, Artificial Sequence
  Synthetic
caggaagtacagacagagaacgtgacagtggctgagggtggggtggctgagatcacctgccgtctgcaccagtatgatgggtccatagttgtcatccagaacccagcccggcagaccctcttcttcaatggcacccgtgccttgaaggatgagcgtttccagcttgaggagttctccccacgccgggtgcggatccggctctcagatgcccgcctggaggacgaggggggctatttctgccagctctacacagaagacacccaccaccagattgccacgctcacggtactagtggccccagagaatcctgtggtggaggtccgggagcaggcggtagagggcggcgaggtggagctcagctgcctcgttccgcggtcccgtccggctgccaccctgcgctggtaccgggaccgcaaggagctgaaaggagtgagcagcagccaggaaaatggcaaggtctggagcgtggcaagcacagtacggtttcgtgtggaccgtaaggacgacggtggtatcatcatctgtgaggcgcagaaccaggcgctgccctccggacacagcaagcagacgcagtacgtgctggatgtgcagtactcccccacggcccggattcatgcctcccaagctgtggtgagggagggagacacgctggtgttgacgtgtgctgtcacggggaaccccaggccaaaccagatccgctggaaccgcgggaatgagtctttgccggagagggcggaggccgtgggagagacgctcacgctgccgggtctggtatccgcggataacggcacctacacttgcgaggcgtccaataagcacggc

SEQ ID NO: 52 - DNA, 69 bp, Artificial Sequence
  Synthetic
gccattgtgggcggcatcctggcgctgctggtgtttctgatcatatgtgtgctagtgggcatggtctgg

SEQ ID NO: 53 - DNA, 126 bp, Artificial Sequence
  Synthetic
tgctcggtacggcagaagggttcctatctgacccacgaagccagtggcttggatgaacagggagaagcaagagaagccttcctcaatggcagcgacggacacaagaggaaagaggaattcttcatc

SEQ ID NO: 54 - DNA, 72 bp, Artificial Sequence
  Synthetic
atgggccgggcccggcgcttccagtggccgctgctgctgctgtgggcggccgcggcggggccaggggcagga

SEQ ID NO: 55 - DNA, 21 bp, Artificial Sequence
  Synthetic
ctatagtcgtcattcagaatt

SEQ ID NO: 56 - DNA, 21 bp, Artificial Sequence
  Synthetic
gcccaaggccgatgaaatctt

SEQ ID NO: 57 - DNA, 19 bp, Artificial Sequence
  Synthetic
ccagcagactctatacttt

SEQ ID NO: 58 - DNA, 20 bp, Artificial Sequence
  Synthetic
ctcggaatcccacagaaacc

SEQ ID NO: 59 - DNA, 19 bp, Artificial Sequence
  Synthetic
gctgagaggtggatctgtc

SEQ ID NO: 60 - DNA, 20 bp, Artificial Sequence
  Synthetic
cgatatccagaaagacacgg

SEQ ID NO: 61 - DNA, 21 bp, Artificial Sequence
  Synthetic
ctgcacttctagataccgctg

SEQ ID NO: 62 - DNA, 21 bp, Artificial Sequence
  Synthetic
cttttccacaagaaggacagg

SEQ ID NO: 63 - DNA, 20 bp, Artificial Sequence
  Synthetic
ctgcgctgctttgaccaatg

SEQ ID NO: 64 - DNA, 19 bp, Artificial Sequence
  Synthetic
cgtggaaatgggaattctg

SEQ ID NO: 65 - DNA, 20 bp, Artificial Sequence
  Synthetic
ggtcaagatggagggaaggg

SEQ ID NO: 66 - DNA, 20 bp, Artificial Sequence
  Synthetic
agagagtctgctaacaggag

SEQ ID NO: 67 - DNA, 20 bp, Artificial Sequence
  Synthetic
ggtatgtggtattgatgtgc

SEQ ID NO: 68 - DNA, 25 bp, Artificial Sequence
  Synthetic
gagcaccctgtgctgctcaccgagg

SEQ ID NO: 69 - DNA, 25 bp, Artificial Sequence
  Synthetic
gtggtggtgaagctgtagccacgct

SEQ ID NO: 70 - DNA, 17 bp, Artificial Sequence
  Synthetic
gaaggctgccgaccccg

SEQ ID NO: 71 - DNA, 18 bp, Artificial Sequence
  Synthetic
ggcgcgcagctgtaaacg

SEQ ID NO: 72 - DNA, 1040 bp, Artificial Sequence
  Synthetic
gggccaatctttcccaggacggctactggcaggagcaggatttggagctgggaactccggctccactggaggaggccctcagctccacagtctggagcaaccctgacctgctggccagtcaagatagccagccctggacgtctgatgaaacagtggtggctggtggcacagtagtgctcaagtgccaagtgaaagaccatgaagactcatctctgcagtggtctaaccctgcccagcagactctatactttggggagaaaagagcccttcgagataatcggattcagctggttagctccaccccgcatgagctcagcatcagcatcagcaacgtggcactggccgacgagggcgagtacacatgctccatcttcactatgcctgtgcggaccgccaagtccctcgtcactgtgctcggaatcccacagaaacccataatcactggttataagtcatcgttgcgggaaaaggagacagccactctaaattgtcagtcttctgggagcaaacctgcagcccagctcgcctggagaaaaggtgaccaagaactccacggggaccagacgcgaatccaggaagatcccaatgggaaaaccttcactgtgagcagctcggtgtcattccaggttacccgggatgatgatggagcaaacgtcgtgtgctctgtgaaccatgaatctctgaagggagctgacagatccacctctcagcgcattgaagtgttatacacaccaacagccatgattaggccagaacctgctcatcctcgtgaaggccagaagctgttgttacattgtgaggggcgtggcaatccagtccctcagcagtacgtgtgggtaaaagaaggcagcgagccacccctcaagatgacccaagagagtgcactcatcttcccatttttgaacaaaagtgacagtggcacctatggctgtacagccacgagcaacatgggcagctatacagcctacttcactctcaatgtcaacgaccctagtccagtgccctcatcctccagtacttaccacgccatcattggaggg

SEQ ID NO: 73 - DNA, 1091 bp, Artificial Sequence
  Synthetic
acaggtgatgggcagaatctgtttactaaggacgtgacggtgattgaaggagaagttgcaaccatcagctgtcaagtcaataagagtgatgactccgtgattcagctactgaaccccaacaggcagaccatttacttcagggacttcaggcctttgaaggacagcaggtttcagctgctgaatttttctagcagtgaactcaaagtgtcactgacaaatgtctcaatttcggatgaagggaggtacttctgccagctctacacggaccctccgcaggagagttataccaccatcacagtcctggtccctccacgtaacttgatgatcgatatccagaaagacacggcagttgaaggggaggagattgaagtcaactgcacggccatggccagcaagccggctacgaccatcaggtggttcaaagggaacaaggaactcaaaggcaaatcagaggtggaagagtggtcggacatgtacactgtgaccagtcagctgatgctgaaggtgcacaaggaggatgatggagtcccagtgatctgccaggtggagcaccctgcggtcactggaaacctgcagacccagcggtatctagaagtgcagtataaaccgcaagtgcagatccagatgacttatcctctgcaaggcctaacccgggaaggggatgcattcgagttaacgtgtgaagccaccgggaagccccagcctgtgatggtaacttgggtgagagtcgatgacgaaatgcctcaacatgccgttctgtctgggccgaatctgttcatcaataacctaaacaaaacagacaatggtacttaccgctgtgaggcttccaacacagtggggaaagctcattcggactatatgctgtatgtatacgatccccccacaactatccctcctcccacaacaaccaccaccaccactaccaccaccaccaccaccaccaccatccttaccatcatcacagacaccacggcgacgacagaaccagcagttcacggccttactcagttgcccaattccgcagaagaactggacagtgaggatctctcagattctcgagcaggtgaagagggcgccattggggcagtgga

SEQ ID NO: 74 - DNA, 1064 bp, Artificial Sequence
  Synthetic
ttcgctcaaccagcatctccttgccccctcgatccttccccagacccttaaaagaagggaccatgatttggaaacgcagcgcggttctccgcttctacagtgtctgcgggctcctgttacaagcggctgcttcaaaaagtaaagtcaaaggcagccaagggcagtttccactaactcagaatgtcacggttgttgaaggtggaactgcaattttgacctgcagagttgatcaaaatgataacacctccctccagtggtcaaatccagctcaacagactctgtacttcgatgacaagaaagctttaagggacaatcggatcgagctggttcgcgcttcttggcatgagttgagcatcagtgtcagtgatgtgtctctctcggatgaaggacagtacacctgctccttatttacaatgcctgtcaaaacttccaaggcctatctcactgtcctgggtgttccggagaagcctcagattagtggattttcgtcacccgtcatggagggagacctgatgcagctgacgtgtaagacgtccggcagtaagcctgcggctgatataagatggttcaaaaatgacaaagaaatcaaagatgtgaagtacttgaaagaggaggatgctaaccgaaagaccttcaccgttagcagcacgctggatttccgagtggaccgtagcgatgatggagtggcggtcatctgcagagtagaccatgagtccctcaatgccacccctcaggtagccatgcaggtgctagaaattcactatacaccatcagttaagattataccatcgactccttttccacaagaagggcaggctttaacgttgacctgtgaatctaaaggaaaaccactgccagaacctgttttgtggacgaaagatggtgccgaattaccagatcctgatcgaatggttgtgagtggtagggaactcaacattctcttcctgaacaaaacggacaatggcacttaccgatgtaaagccactaacaccattggccaaagcagtgccgagtatgtcctcattgttcacgatcccaattctctggctggccagaatggccct

SEQ ID NO: 75 - DNA, 1608 bp, Artificial Sequence
  Synthetic
gagggtggggtggctgagatcacctgccgtctgcatcagtatgatgggtctatagtcgtcattcagaacccagcccggcagaccctctttttcaatggcacccgagccctgaaggacgaacgattccagctggaggagttctccccgcgccgagtgcgcatcaggctctcagacgcccgcctggaggacgaggggggctacttctgccagctctacacggaggacacccaccaccagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgcgatcgcaaggagctgaaaggagtgagcagcggccaggagaacggcaaggtgtggagcgtggcgagcactgtgcggttccgcgtggaccgcaaggacgacggcggtatcgtcatctgtgaagcgcagaaccaggcgctgccctcggggcacagcaagcagacgcagtacgtgctggatgtgcagtattcccccacagctcggatccacgcctctcaagctgtagtcagggagggagacactctggtgctgacatgtgctgtcacagggaaccccaggccaaaccagatccgctggaaccgtgggaatgagtctctgccagagagggcggaggcgttgggcgagacgctcacgctgcctggcctggtatctgcagataacggcacctacacctgcgaggcggcgaacaagcacggccacgccagggcgctctacgtgcttgtggtctatgaccccggagcagtggtagaggctcagacatcggttccctacgccatcgtgggcggcatcctggcgctactggtgtttctgatcatatgcgtgctagtaggcatggtgtggtgctctgtccgacagaagggctcctatctgacccatgaggccagtggcttggatgagcagggagaagccagagaagccttcctcaacggcagtgatggacacaagcggaaagaagaattcttcatttgaccccactcccccactgccagctgcaaggaaccagcaaagacattgaccagagtccgggatggtgggcttcccccccactagctaacacctcaggcgcttgggcagggattgggggtgttggatgcctggatctctcccagggccaaaggctagggtccagagacttgggatcccctagggagacagggacttaacgtcccttctagaccctgagtccagggacgggtaatagggatttgggttgggggaagaaaacagggggaaggccagaacccctagactacagaaccaggtcttgtgggggagggggtcagattctgggaagggtgggtgggcagggaagggaaacacagatttctttggggtcccagaccccatgccagccattgtaagagttccacagagctctgggcacctctttgcaaagccatgtttgcacggtggggggaggggcaggggtgggggcagtaagggagacaaccccgacccttatccagtccccttgctccgggttcctgggcggctccccttccctccatcttgacc

SEQ ID NO: 76 - DNA, 1326 bp, Artificial Sequence
  Synthetic
gggaggtggccaggaagcgcgatgggggccccttccgccctgcccctgctcctgctcctcgcctgctcctgggcgcccggcggggccaatctttcccaggacggctactggcaggagcaggatttggagctgggaactccggctccactggaggaggccctcagctccacagtctggagcaaccctgacctgctggccagtcaagatagccagccctggacgtctgatgaaacagtggtggctggtggcacagtagtgctcaagtgccaagtgaaagaccatgaagactcatctctgcagtggtctaaccctgcccagcagactctatactttggggagaaaagagcccttcgagataatcggattcagctggttagctccaccccgcatgagctcagcatcagcatcagcaacgtggcactggccgacgagggcgagtacacatgctccatcttcactatgcctgtgcggaccgccaagtccctcgtcactgtgctcggaatcccacagaaacccataatcactggttataagtcatcgttgcgggaaaaggagacagccactctaaattgtcagtcttctgggagcaaacctgcagcccagctcgcctggagaaaaggtgaccaagaactccacggggaccagacgcgaatccaggaagatcccaatgggaaaaccttcactgtgagcagctcggtgtcattccaggttacccgggatgatgatggagcaaacgtcgtgtgctctgtgaaccatgaatctctgaagggagctgacagatccacctctcagcgcattgaagtgttatacacaccaacagccatgattaggccagaacctgctcatcctcgtgaaggccagaagctgttgttacattgtgaggggcgtggcaatccagtccctcagcagtacgtgtgggtaaaagaaggcagcgagccacccctcaagatgacccaagagagtgcactcatcttcccatttttgaacaaaagtgacagtggcacctatggctgtacagccacgagcaacatgggcagctatacagcctacttcactctcaatgtcaacgaccctagtccagtgccctcatcctccagtacttaccacgccatcattggagggatcgtggctttcatcgtcttcctgctgctcattctgctcattttccttggacactatttgatccggcacaaaggaacgtacctgacacacgaagcgaagggctccgacgacgccccagatgcagatacggccatcatcaacgcagaaggcgggcagtcaggcggggatgacaagaaggaatatttcatctaggggcacccacac

SEQ ID NO: 77 - DNA, 1308 bp, Artificial Sequence
  Synthetic
atgatttggaaacgcagcgcggttctccgcttctacagtgtctgcgggctcctgttacaagcggctgcttcaaaaagtaaagtcaaaggcagccaagggcagtttccactaactcagaatgtcacggttgttgaaggtggaactgcaattttgacctgcagagttgatcaaaatgataacacctccctccagtggtcaaatccagctcaacagactctgtacttcgatgacaagaaagctttaagggacaatcggatcgagctggttcgcgcttcttggcatgagttgagcatcagtgtcagtgatgtgtctctctcggatgaaggacagtacacctgctccttatttacaatgcctgtcaaaacttccaaggcctatctcactgtcctgggtgttccggagaagcctcagattagtggattttcgtcacccgtcatggagggagacctgatgcagctgacgtgtaagacgtccggcagtaagcctgcggctgatataagatggttcaaaaatgacaaagaaatcaaagatgtgaagtacttgaaagaggaggatgctaaccgaaagaccttcaccgttagcagcacgctggatttccgagtggaccgtagcgatgatggagtggcggtcatctgcagagtagaccatgagtccctcaatgccacccctcaggtagccatgcaggtgctagaaattcactatacaccatcagttaagattataccatcgactccttttccacaagaagggcaggctttaacgttgacctgtgaatctaaaggaaaaccactgccagaacctgttttgtggacgaaagatggtgccgaattaccagatcctgatcgaatggttgtgagtggtagggaactcaacattctcttcctgaacaaaacggacaatggcacttaccgatgtaaagccactaacaccattggccaaagcagtgccgagtatgtcctcattgttcacgatcccaattctctggctggccagaatggccctgaccatgctctcatcggaggaatagtggctgttgttgtatttgttacactgtgctccatattcctgcttggccgatatctggcaagacataaaggaacatatctaacaaatgaagctaaaggagccgaagatgcaccagatgccgacacagccattatcaatgctgaaggcagccaagtcaatgccgaagagaaaaaagagtatttcattggatccgagctcggtaccaagcttgggcccgaacaaaaactcatctcagaagaggatctgaatagcgccgtcgaccatcatcatcatcatcattga

SEQ ID NO: 78 - DNA, 1167 bp, Artificial Sequence
  Synthetic
atgggccgggcccggcgcttccagtggccgctgctgctgctgtgggcggccgcggcggggccagggacgggacaggaagtacagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagcacggccacgccagggcgctctacgtgcttgtggtctatgaccccggagcagtggtagaggctcagacatcggttccctacgccatcgtgggcggcatcctggcgctactggtgtttctgatcatatgcgtgctagtaggcatggtgtggtgctctgtccgacagaagggctcctatctgacccatgaggccagtggcttggatgagcagggagaagccagagaagccttcctcaacggcagtgatggacacaagcggaaagaagaattcttcatttga

SEQ ID NO: 79 - DNA, 6717 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcaagcttgccaccatgggccgggcccggcgcttccagtggccgctgctgctgctgtgggcggccgcggcggggccagggacgggacaggaagtacagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagcacggccacgccagggcgctctacgtgcttgtggtctatgaccccggagcagtggtagaggctcagacatcggttccctacgccatcgtgggcggcatcctggcgctactggtgtttctgatcatatgcgtgctagtaggcatggtgtggtgctctgtccgacagaagggctcctatctgacccatgaggccagtggcttggatgagcagggagaagccagagaagccttcctcaacggcagtgatggacacaagcggaaagaagaattcttcatttgaccccactcccccactgccagctgcaaggagccagcctcgagcggccgccactgtgctggatatctgcagaattccaccacactggactagtggatccgagctcggtaccaagcttgggcccgaacaaaaactcatctcagaagaggatctgaatagcgccgtcgaccatcatcatcatcatcattgagtttaaacggtctccagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 80 - DNA, 1731 bp, Artificial Sequence
  Synthetic
atggagacagacacactcctgctatgggtactgctgctctgggttccaggttccactggtgacgcggcccagccggccaatctttcccaggacgatagccagccctggacgtctgatgaaacagtggtggctggtggcacagtagtgctcaagtgccaagtgaaagaccatgaagactcatctctgcagtggtctaaccctgcccagcagactctatactttggggagaaaagagcccttcgagataatcggattcagctggttagctccaccccgcatgagctcagcatcagcatcagcaacgtggcactggccgacgagggcgagtacacatgctccatcttcactatgcctgtgcggaccgccaagtccctcgtcactgtgctcggaatcccacagaaacccataatcactggttataagtcatcgttgcgggaaaaggagacagccactctaaattgtcagtcttctgggagcaaacctgcagcccagctcgcctggagaaaaggtgaccaagaactccacggggaccagacgcgaatccaggaagatcccaatgggaaaaccttcactgtgagcagctcggtgtcattccaggttacccgggatgatgatggagcaaacgtcgtgtgctctgtgaaccatgaatctctgaagggagctgacagatccacctctcagcgcattgaagtgttatacacaccaacagccatgattaggccagaacctgctcatcctcgtgaaggccagaagctgttgttacattgtgaggggcgtggcaatccagtccctcagcagtacgtgtgggtaaaagaaggcagcgagccacccctcaagatgacccaagagagtgcactcatcttcccatttttgaacaaaagtgacagtggcacctatggctgtacagccacgagcaacatgggcagctatacagcctacttcactctcaatgtcaacgaccctagtccagtgccctcatcctccagtacggatccccgtcgtgcatctatcgaaggtcgtggagatcccgaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 81 - DNA, 1728 bp, Artificial Sequence
  Synthetic
atggagacagacacactcctgctatgggtactgctgctctgggttccaggttccactggtgacgcggcccagccggcccccacaggtgatggacagaatctgtttactaaagacgtgacagtgattgaaggagaagtggcaaccatcagctgccaggtcaataagagtgacgactcagtgatccagctcctgaaccccaacaggcagaccatttacttcagggacttcaggcctttgaaggacagcaggtttcagctgctgaatttttctagcagtgaactcaaagtgtcactgacgaatgtctcaatctcggatgaagggagatacttctgccagctctacacggaccccccacaggagagttacaccaccatcacagtcctggttcctccacgtaacttgatgatcgatatccagaaagacacggcagttgaaggggaggagattgaagtcaactgtactgccatggccagcaagccagcgacgaccatcaggtggttcaaagggaacaaggaactcaaaggcaaatcagaggtggaggagtggtcggacatgtacactgtgaccagtcagctgatgctgaaggtgcacaaggaggacgacggggtcccggtgatctgccaggtggagcaccctgcggtcactggaaacctgcagacccagcgctatctagaagtgcagtataaaccgcaagtgcatatccagatgacttaccctctgcaaggcctaacccgggaaggggatgcatttgagttaacgtgtgaagccatcgggaagccccagcctgtgatggtaacttgggtgagagtcgatgatgaaatgcctcaacatgccgtactgtctgggccaaacctgttcatcaataacctaaacaaaacagataacggtacttaccgctgtgaggcttccaacatagtgggaaaggctcattcggactatatgctgtatgtatacgattctcgagcaggtgaagaggggaccattggggatccccgtcgtgcatctatcgaaggtcgtggagatcccgaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 82 - DNA, 1743 bp, Artificial Sequence
  Synthetic
atgatttggaaacgcagcgcggttctccgcttctacagtgtctgcgggctcctgttacaagcggctgcttcaaaaagtaaagtcaaaggcagccaagggcagtttccactaactcagaatgtcacggttgttgaaggtggaactgcaattttgacctgcagagttgatcaaaatgataacacctccctccagtggtcaaatccagctcaacagactctgtacttcgatgacaagaaagctttaagggacaatcggatcgagctggttcgcgcttcttggcatgagttgagcatcagtgtcagtgatgtgtctctctcggatgaaggacagtacacctgctccttatttacaatgcctgtcaaaacttccaaggcctatctcactgtcctgggtgttccggagaagcctcagattagtggattttcgtcacccgtcatggagggagacctgatgcagctgacgtgtaagacgtccggcagtaagcctgcggctgatataagatggttcaaaaatgacaaagaaatcaaagatgtgaagtacttgaaagaggaggatgctaaccgaaagaccttcaccgttagcagcacgctggatttccgagtggaccgtagcgatgatggagtggcggtcatctgcagagtagaccatgagtccctcaatgccacccctcaggtagccatgcaggtgctagaaattcactatacaccatcagttaagattataccatcgactccttttccacaagaagggcaggctttaacgttgacctgtgaatctaaaggaaaaccactgccagaacctgttttgtggacgaaagatggtgccgaattaccagatcctgatcgaatggttgtgagtggtagggaactcaacattctcttcctgaacaaaacggacaatggcacttaccgatgtaaagccactaacaccattggccaaagcagtgccgagtatgtcctcattgttcacgatcccaattctctggctggccagaatggcccggatccccgtcgtgcatctatcgaaggtcgtggagatcccgaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 83 - DNA, 1707 bp, Artificial Sequence
  Synthetic
atgggccgggcccggcgcttccagtggccgctgctgctgctgtgggcggccgcggcggggccagggacgggacaggaagtacagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagcacggccacgcgagggcgctctacgtgcttgtggtctatgaccccggtgcagtggtagaggctcagacatcggttccagatccccgtcgtgcatctatcgaaggtcgtggagatcccgaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 84 - DNA, 6658 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcaagcttgcggcggcggcggcggcaccatgggccgggcccggcgcttccagtggccgctgctgctgctgtgggcggccgcggcggggccagggacgggacaggaagtacagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagcacggccacgcgagggcgctctacgtgcttgtggtctatgaccccggtgcagtggtagaggctcagacatcggttccagatccccgtcgtgcatctatcgaaggtcgtggagatcccgaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 85 - DNA, 846 bp, Artificial Sequence
  Synthetic
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattcggacactatttgatccggcacaaaggaacgtacctgacacacgaagcgaagggctccgacgacgccccagatgcagatacggccatcatcaacgcagaaggcgggcagtcaggcggggatgacaagaaggaatatttcatctag

SEQ ID NO: 86 - DNA, 852 bp, Artificial Sequence
  Synthetic
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattcattctgggccgctattttgccagacataaaggtacatacttcactcatgaagccaaaggagccgatgacgcagcagacgcagacacagctataatcaatgcagaaggaggacagaacaactccgaagaaaagaaagagtacttcatctag

SEQ ID NO: 87 - DNA, 852 bp, Artificial Sequence
  Synthetic
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattcctgcttggccgatatctggcaagacataaaggaacatatctaacaaatgaagctaaaggagccgaagatgcaccagatgccgacacagccattatcaatgctgaaggcagccaagtcaatgccgaagagaaaaaagagtatttcatttaa

SEQ ID NO: 88 - DNA, 831 bp, Artificial Sequence
  Synthetic
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattctgctctgtccgacagaagggctcctatctgacccatgaggccagtggcttggatgagcagggagaagccagagaagccttcctcaacggcagtgatggacacaagcggaaagaagagttcttcatttga

SEQ ID NO: 89 - DNA, 5104 bp, Artificial Sequence
  Synthetic
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattctgctctgtccgacagaagggctcctatctgacccatgaggccagtggcttggatgagcagggagaagccagagaagccttcctcaacggcagtgatggacacaagcggaaagaagagttcttcatttgactcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 90 - DNA, 867 bp, Artificial Sequence
  Synthetic
atggtgagcaagcagatcctgaagaacaccggcctgcaggagatcatgagcttcaaggtgaacctggagggcgtggtgaacaaccacgtgttcaccatggagggctgcggcaagggcaacatcctgttcggcaaccagctggtgcagatccgcgtgaccaagggcgcccccctgcccttcgccttcgacatcctgagccccgccttccagtacggcaaccgcaccttcaccaagtaccccgaggacatcagcgacttcttcatccagagcttccccgccggcttcgtgtacgagcgcaccctgcgctacgaggacggcggcctggtggagatccgcagcgacatcaacctgatcgaggagatgttcgtgtaccgcgtggagtacaagggccgcaacttccccaacgacggccccgtgatgaagaagaccatcaccggcctgcagcccagcttcgaggtggtgtacatgaacgacggcgtgctggtgggccaggtgatcctggtgtaccgcctgaacagcggcaagttctacagctgccacatgcgcaccctgatgaagagcaagggcgtggtgaaggacttccccgagtaccacttcatccagcaccgcctggagaagacctacgtggaggacggcggcttcgtggagcagcacgagaccgccatcgcccagctgaccagcctgggcaagcccctgggcagcctgcacgagtgggtgtccggactcagatctgaattctgctctgtccgacagaagggctcctatctgacccatgaggccagtggcttggatgagcagggagaagccagagaagccttcctcaacggcagtgatggacacaagcggaaagaagagttcttcatttga

SEQ ID NO: 91 - DNA, 5377 bp, Artificial Sequence
  Synthetic
ctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaagcggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccaggtcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatccgtcgccaccatggtgagcaagcagatcctgaagaacaccggcctgcaggagatcatgagcttcaaggtgaacctggagggcgtggtgaacaaccacgtgttcaccatggagggctgcggcaagggcaacatcctgttcggcaaccagctggtgcagatccgcgtgaccaagggcgcccccctgcccttcgccttcgacatcctgagccccgccttccagtacggcaaccgcaccttcaccaagtaccccgaggacatcagcgacttcttcatccagagcttccccgccggcttcgtgtacgagcgcaccctgcgctacgaggacggcggcctggtggagatccgcagcgacatcaacctgatcgaggagatgttcgtgtaccgcgtggagtacaagggccgcaacttccccaacgacggccccgtgatgaagaagaccatcaccggcctgcagcccagcttcgaggtggtgtacatgaacgacggcgtgctggtgggccaggtgatcctggtgtaccgcctgaacagcggcaagttctacagctgccacatgcgcaccctgatgaagagcaagggcgtggtgaaggacttccccgagtaccacttcatccagcaccgcctggagaagacctacgtggaggacggcggcttcgtggagcagcacgagaccgccatcgcccagctgaccagcctgggcaagcccctgggcagcctgcacgagtgggtgtccggactcagatctgaattctgctctgtccgacagaagggctcctatctgacccatgaggccagtggcttggatgagcagggagaagccagagaagccttcctcaacggcagtgatggacacaagcggaaagaagagttcttcatttgactcgagtagtcgacgataaaataaaagattttatttagtctccagaaaaaggggggaatgaaagaccccacctgtaggtttggcaagctagcttaagtaacgccattttggaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 92 - DNA, 1230 bp, Artificial Sequence
  Synthetic
atggagacagacacactcctgctatgggtactgctgctctgggttccaggttccactggtgacgcggcccagccggccaggcgcgccgtacgaagcttggatcccgaacaaaaactcatctcagaagaggatctgagcatgatgcagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagcacggccacgcgagggcgctctacgtgcttgtggtctatgaccccggtgcagtggtagaggctcagacatcggttccttacgccattgtgggcggcatcctggcgctactggtgtttctgatcatatgtgtgctagtgggcatggtctggtgctccgtccgacagaagggatcctatctgacccatgaggccagtggcttggatgagcagggagaagccagagaagccttcctcaatggcggcgatggacacaaacggaaagaagaattcttcatttga

SEQ ID NO: 93 - DNA, 5796 bp, Artificial Sequence
  Synthetic
ctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaagcggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccaggtcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatcccagtgtggtggtacgggaattcgtcgccaccatggagacagacacactcctgctatgggtactgctgctctgggttccaggttccactggtgacgcggcccagccggccaggcgcgccgtacgaagcttggatcccgaacaaaaactcatctcagaagaggatctgagcatgatgcagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagcacggccacgcgagggcgctctacgtgcttgtggtctatgaccccggtgcagtggtagaggctcagacatcggttccttacgccattgtgggcggcatcctggcgctactggtgtttctgatcatatgtgtgctagtgggcatggtctggtgctccgtccgacagaagggatcctatctgacccatgaggccagtggcttggatgagcagggagaagccagagaagccttcctcaatggcggcgatggacacaaacggaaagaagaattcttcatttgaccctacttccccaccgccagctgcaaggagccagcctcgagtcgtcgacgataaaataaaagattttatttagtctccagaaaaaggggggaatgaaagaccccacctgtaggtttggcaagctagcttaagtaacgccattttgaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 94 - DNA, 1101 bp, Artificial Sequence
  Synthetic
atggagacagacacactcctgctatgggtactgctgctctgggttccaggttccactggtgacgcggcccagccggccaggcgcgccgtacgaagcttggatcccgaacaaaaactcatctcagaagaggatctgagcatgcagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagcacggccacgcgagggcgctctacgtgcttgtggtctatgaccccggtgcagtggtagaggctcagacatcggttccttacgccattgtgggcggcatcctggcgctactggtgtttctgatcatatgtgtgctagtgggcatggtctggtag

SEQ ID NO: 95 - DNA, 5633 bp, Artificial Sequence
  Synthetic
ctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaagcggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccaggtcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatcccagtgtggtggtacgggaattcgtcgccaccatggagacagacacactcctgctatgggtactgctgctctgggttccaggttccactggtgacgcggcccagccggccaggcgcgccgtacgaagcttggatcccgaacaaaaactcatctcagaagaggatctgagcatgcagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagcacggccacgcgagggcgctctacgtgcttgtggtctatgaccccggtgcagtggtagaggctcagacatcggttccttacgccattgtgggcggcatcctggcgctactggtgtttctgatcatatgtgtgctagtgggcatggtctggtagccctcgaggtcgacgataaaataaaagattttatttagtctccagaaaaaggggggaatgaaagaccccacctgtaggtttggcaagctagcttaagtaacgccattttggaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 96 - DNA, 1167 bp, Artificial Sequence
  Synthetic
atgggccgggcccggcgcttccagtggccgctgctgctactgtgggcggccgcggcggggccagggacggcacaggaagtgcagacagagaatgtgacggtggccgagggtggggtggctgagatcacctgccgtctgcatcagtatgatgggtctatagtcgtcattcagaacccagcccggcagaccctctttttcaatggcacccgagccctgaaggacgaacgattccagctggaggagttctccccgcgccgagtgcgcatcaggctctcagacgcccgcctggaggacgaggggggctacttctgccagctctacacggaggacacccaccaccagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgcgatcgcaaggagctgaaaggagtgagcagcggccaggagaacggcaaggtgtggagcgtggcgagcactgtgcggttccgcgtggaccgcaaggacgacggcggtatcgtcatctgtgaagcgcagaaccaggcgctgccctcggggcacagcaagcagacgcagtacgtgctggatgtgcagtattcccccacagctcggatccacgcctctcaagctgtagtcagggagggagacactctggtgctgacatgtgctgtcacagggaaccccaggccaaaccagatccgctggaaccgtgggaatgagtctctgccagagagggcggaggcgttgggcgagacgctcacgctgcctggcctggtatctgcagataacggcacctacacctgcgaggcggcgaacaagcacggccacgccagggcgctctacgtgcttgtggtctatgaccccggagcagtggtagaggctcagacatcggttccctacgccatcgtgggcggcatcctggcgctactggtgtttctgatcatatgcgtgctagtaggcatggtgtggtgctctgtccgacagaagggctcctatctgacccatgaggccagtggcttggatgagcagggagaagccagagaagccttcctcaacggcagtgatggacacaagcggaaagaagaattcttcatttga

SEQ ID NO: 97 - DNA, 2472 bp, Artificial Sequence
  Synthetic
atgtgggatgggcacggtgagaaacagccctgcaaattctcccttgcaaacatcagctttgtgaaaggactttctttagaaccggaagccgttggcagctgggaagttgaggatgtgaaggtgagaatgttcgggcgctgcctgctgatccagcacgtggaaagtctcctgcagcttccaaagagtgctccttgctgtggggcatctagcaaaggtatcttctgcatccccgagtggagagaacactactctgcccggagagaagccgagacactcatacgggaaaatccagcaaacatctgcgtgcaggtgacagcagcttgctgtgccagtgacctacagaactctcacctccattcgatgacccaccgaggagcagaggtgaacgaagaacccatgggcctcctgaggcggcggctgctgctgctggtggtggtggtgacgacctgtgtcccagcctcccagggcctgcggtgcatacagtgcgaaagtaaccaggactgcctggtggaggagtgtgccctgggccaggacctctgcagaaccacggtgcttcgggaatgggaagggacggcacaggaagtgcagacagagaatgtgacggtggccgagggtggggtggctgagatcacctgccgtctgcatcagtatgatgggtctatagtcgtcattcagaacccagcccggcagaccctctttttcaatggcacccgagccctgaaggacgaacgattccagctggaggagttctccccgcgccgagtgcgcatcaggctctcagacgcccgcctggaggacgaggggggctacttctgccagctctacacggaggacacccaccaccagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgcgatcgcaaggagctgaaaggagtgagcagcggccaggagaacggcaaggtgtggagcgtggcgagcactgtgcggttccgcgtggaccgcaaggacgacggcggtatcgtcatctgtgaagcgcagaaccaggcgctgccctcggggcacagcaagcagacgcagtacgtgctggatgtgcagtattcccccacagctcggatccacgcctctcaagctgtagtcagggagggagacactctggtgctgacatgtgctgtcacagggaaccccaggccaaaccagatccgctggaaccgtgggaatgagtctctgccagagagggcggaggcgttgggcgagacgctcacgctgcctggcctggtatctgcagataacggcacctacacctgcgaggcggcgaacaagcacggccacgccagggcgctctacgtgcttgtggtctatgaccccggagcagtggtagaggctcagacatcggttccctacgccatcgtgggcggcatcctggcgctactggtgtttctgatcatatgcgtgctagtaggcatggtgtggtgctctgtccgacagaagggctcctatctgacccatgaggccagtggcttggatgagcagggagaagccagagaagccttcctcaacggcagtgatggacacaagcggaaagaagaattcttcatttgaccccactcccccactgccagctgcaaggaaccagcaaagacattgaccagagtccgggatggtgggcttcccccccactagctaacacctcaggcgcttgggcagggattgggggtgttggatgcctggatctctcccagggccaaaggctagggtccagagacttgggatcccctagggagacagggacttaacgtcccttctagaccctgagtccagggacgggtaatagggatttgggttgggggaagaaaacagggggaaggccagaacccctagactacagaaccaggtcttgtgggggagggggtcagattctgggaagggtgggtgggcagggaagggaaacacagatttctttggggtcccagaccccatgccagccattgtaagagttccacagagctctgggcacctctttgcaaagccatgtttgcacggtggggggaggggcaggggnnnnnnnnnnnnnnnnnnnnnnnnnnnnnnnnnnnnnnnnnnnnnnnntgggggcagtaagggagacaaccccgacccttatccagtccccttgctccgggttcctgggcggctccccttccctccatcttgacctccatgtccttcccatcccataattgtctgttcacccgctcttgggggggccttcccagtctctcctccaggccccccggggagggggaaaggagtttggaggggatgtgtggtctctatggttttatatataatatattaaaaaatcaaaaatctgtatgaaaaatatcagattgcacccctctccccccattactggcttttgcc

SEQ ID NO: 98 - protein, 323 aa, Rattus norvegicus
MGRARRFQWPLLLLWAAAAGPGTAQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAAVLRWYRDRKELKGVSSGQENGKVWSVASTVRFRVDRKDDGGIVICEAQNQALPSGHSKQTQYVLDVQYSPTARIHASQAVVREGDTLVLTCAVTGNPRPNQIRWNRGNESLPERAEALGETLTLPGLVSADNGTYTCEAANKHGHARALYVLVVYDPGAVVEAQTSVPY

SEQ ID NO: 99 - protein, 299 aa, Rattus norvegicus
QEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAAVLRWYRDRKELKGVSSGQENGKVWSVASTVRFRVDRKDDGGIVICEAQNQALPSGHSKQTQYVLDVQYSPTARIHASQAVVREGDTLVLTCAVTGNPRPNQIRWNRGNESLPERAEALGETLTLPGLVSADNGTYTCEAANKHGHARALYVLVVYDPGAVVEAQTSVPY

SEQ ID NO: 100 - protein, 42 aa, Rattus norvegicus; identical to SEQ ID NO: 4

SEQ ID NO: 101 - protein, 23 aa, Rattus norvegicus
AIVGGILALLVFLIICVLVGMVW

SEQ ID NO: 102 - protein, 24 aa, Rattus norvegicus
MGRARRFQWPLLLLWAAAAGPGTA

SEQ ID NO: 103 - DNA, 965 bp, Artificial Sequence
  Synthetic
gggaggtgcaggtgccgggccgggagcgagcggcggcggcggtggcggcggcggtggcggcggcggcggcggcaccatgggccgggcccggcgcttccagtggccgctgctgctgctgtgggcggccgcggcggggccagggacgggacaggaagtacagaccgagaatgtgactgtggctgagggtggggtggctgagatcacctgccgtctgcatcagtacgatgggtccatagtcgtcattcagaacccagcccggcagaccctctttttcaatggcaccagagctctgaaggacgagcgattccagctggaggagttctccccgcgccgggtgcgcatcaggctctcagacgcccgcctggaggacgaggggggttacttctgccagctctacacggaggacacccaccatcagatcgccacgctcactgtcttagtggctccggagaatcctgtggtggaggtccgagagcaagcagtggagggcggcgaggtggaactcagctgcctggttccgcggtcgcgccccgcagcggtcctgcgctggtatcgggatcgcaaggagttgaaaggagtgagcagcggccaggagaacggcaaggtgtggagcgtggcaagcaccgtgcggttccgtgtggatcgcaaggacgacggtggtattgtcatctgcgaagcacagaaccaggcgctgccctcgggacacagcaagcagacgcagtacgtgctggatgtgcagtattcccccacagcgcggatccacgcctctcaagctgtagtgagggagggagacactctggtgctgacctgtgctgtcacagggaaccccaggccaaaccagatccgctggaaccgtgggaatgagtctctgccagagagggcggaggcggtgggcgagacgctcacgctacctggcctggtatctgcagataacggcacctacacctgcgaggcggcgaacaagc

SEQ ID NO: 104 - DNA, 129 bp, Artificial Sequence
  Synthetic
tgctctgtccgacagaagggctcctatctgacccatgaggccagtggcttggatgagcagggagaagccagagaagccttcctcaacggcagtgatggacacaagcggaaagaagaattcttcatttga